(19) United States
(12) Patent Application Publication
(10) Pub. No.: US 2023/0181721 A1
(43) Pub. Date: Jun. 15, 2023

(54) VACCINE AGAINST SARS-COV VIRUS

(71) Applicant: OSE IMMUNOTHERAPEUTICS, Nantes (FR)

(72) Inventors: DOMINIQUE COSTANTINI, PARIS (FR); ISABELLE GIRAULT, NANTES (FR); NICOLAS POIRIER, GRANDCHAMPS DES FONTAINES (FR); CAROLINE MARY, SAINTE PAZANNE (FR); VANESSA GAUTTIER, REZÉ (FR); AURORE MORELLO, SAINT SEBASTIEN SUR LOIRE (FR)

(21) Appl. No.: 17/924,371

(22) PCT Filed: May 11, 2021
(86) PCT No.: PCT/EP2021/062481

§ 371 Date: Nov. 10, 2022

(30) Foreign Application Priority Data
May 11, 2020 (EP) 20305469.7
Aug. 14, 2020 (EP) 20305930.8
Jan. 20, 2021 (EP) 21305071.9

(51) Int. Cl. A61K 39/215; A61K 39/39; A61P 31/14

(52) U.S. Cl. CPC A61K 39/215; A61K 39/39; A61P 31/14; A61K 2039/70

(57) ABSTRACT

The present invention relates to a vaccine composition and its use against a Severe acute respiratory syndrome-related coronavirus.

### FIELD OF THE INVENTION

The present invention relates to a vaccine against a Severe acute respiratory syndrome-related coronavirus (SARS-CoV) and its use.

### BACKGROUND OF THE INVENTION

In 2020, the COVID-19 pandemic due to the rapid worldwide spread of SARS Cov-2 virus (novel coronavirus outbreak 2019-nCoV) is becoming a serious global health threat. It was declared by the WHO as Public Health Emergency of International Concern (PHEIC). A vaccine is urgently needed trying to stop the global spread and the COVID-19 mortality seen in ICU.

The precedent emergent Coronaviruses (RNA viruses which usually cause mild upper respiratory illnesses) are also causing SARS (severe acute respiratory Syndrome) or MERS (Middle east respiratory syndrome) were causing global attention on the clinical significance of coronaviruses. SARS, which is caused by the SARS coronavirus (SARS-CoV), emerged from China and caused nearly 8500 cases and 916 deaths during the outbreak in 2002 and 2003. Overall fatality of SARS-CoV was about 10% in the general population, but >50% in patients aged 65 years and older (Shibo J et al; Future Virology 2013; “Development of SARS vaccines and therapeutics is still needed”).

A number of inactivated and live-attenuated SARS vaccines, as well as those based on vectors encoding the full-length S protein of SARS-CoV, showed high immunogenicity in inducing neutralizing antibody responses and protection against SARS-CoV challenge. However, most of these vaccine candidates may also induce immunopathology or other harmful immune responses such as antibody-dependent enhancement (ADE) phenomenon (Iwasaki et al. Nature Review Immunology 2020), raising concerns about their safety (Weingartl H, et al J. Virol.-2004. Immunization with modified vaccinia virus Ankara-based recombinant vaccine against severe acute respiratory syndrome is associated with enhanced hepatitis in ferrets).

Virus-like-particle vaccine (whole virus vaccine and an rDNA-produced S protein) induced protection against infection but challenged animals exhibited an immunopathologic-type lung disease (Tseng C T, Sbrana E, Iwata-Yoshikawa N, Newman P C, Garron T, et al. PLOS ONE 2012, Immunization with SARS Coronavirus Vaccines Leads to Pulmonary Immunopathology on Challenge with the SARS Virus). Various approaches (recombinant S protein-based, DNA-based or RNA-based, Viral vector-based, Recombinant RBD protein-based, siRNA, peptides) were explored as candidate vaccines (Du L, He Y, Zhou Y et al. Nat. Rev. Microbiol 2009—The spike protein of SARS-CoV: a target for vaccine and therapeutic development.).

Today, various vaccine technologies from the new SARS-Cov-2 virus are also prepared and will be explored in clinical trials (Le T T et al Nature review 2020—The COVID-19 vaccine development landscape). The most advanced candidates have recently moved into clinical development, including mRNA-1273 (LNP-encapsulated mRNA vaccine encoding S protein—Moderna), Ad5-nCoV—(Adenovirus type 5 vector that expresses S protein—CanSino Biologicals), INO-4800 (DNA plasmid encoding S protein delivered by electroporation—Inovio), LV-SM ENP-DC (Dentritic Cells modified with lentiviral vector expressing synthetic minigene based on domains of selected viral proteins; administered with antigen-specific CTLs—Shenzhen Geno-Immune Medical Institute), specific aAPC (Pathogen-specific aAPC modified with lentiviral vector expressing synthetic minigene based on domains of selected viral proteins—Shenzhen Geno-Immune Medical Institute).

Peptides are also vaccines candidates. Even they are considered as a lower immunogenic strategy, various peptides approaches were studied from the previous SARS-Cov virus spread and are today explored for the new pandemic related to the SARS-COv2 virus (Zheng B J et al; Antiviral Therapy 2005, Synthetic peptides outside the spike protein heptad repeat regions as potent inhibitors of SARS-associated coronavirus). The main research on peptides (fragments of proteins or protein shells that mimic the coronavirus's outer coat) focuses on viral protein subunits—mainly on the virus's spike protein or a key part of it called the receptor binding domain RBD (Callaway E; Nature Vol 580, 30 Apr. 2020, the race for coronavirus vaccine).

As a whole, most candidates presented for which information is available aim to induce neutralizing antibodies against the viral spike (S) protein, preventing uptake via the human ACE2 receptor. However, vaccines based on antibodies produced by B lymphocytes cells are not sufficiently efficient against coronavirus yet. Their action is for the short-term prophylactic response (Wu et al. Emerg Infect Dis. 2007; Ho et al. Emerg Infect Dis. 2005). Besides in some cases and patients, such antibodies may raise a risk of tolerance linked to a problem of increased endocytosis of the virus by cells of the host and/or of excessive inflammatory processes (Iwasaki et al. Nature Review Immunology 2020). Furthermore, previous SARS-COV failed to induce long-term memory B cells (Tang et al. J. Immunol 2011) while memory T cells have been observed up to 11 years after infection (Ng O W Vaccine 2016).

According to another and less known approach, vaccines focusing the activation of T lymphocytes have also been studied: Channappanavar et al. (Journal of Virology 2014 Virus-specific memory CD8 T cells provide substantial protection from lethal SARS-CoV infection); and Tan A C L et al. (Immunol Cell Biol 2013 —The design and proof of concept for a CD8+ T cell-based vaccine inducing cross-subtype protection against influenza A virus). Both teams have demonstrated for other viruses that a peptide vaccine based on two natural CD8 peptides against the SARS-CoV 2003 virus or the Flu Virus induce lasting immunization in mice with in particular the induction of T Resident Memory cells (TRM) in the pulmonary parenchyma and the pulmonary alveoli. Channappanavar was using intravenous peptide-pulsed Dendritic Cells followed by intranasal boosting with recombinant vaccinia virus (rVV) encoding peptides. Tan and collaborators were using three of the natural HLA-A2-restricted influenza epitopes into lipopeptides by intranasal administration.

These peptide vaccines are based on the use of natural, wild-type, naïve epitopes. There is however a need of a better efficiency and in particular of an increased immunogenicity of the vaccine allowing a stronger activation of the immune T cells and preferably CD8 T cells able to destroy cells that are infected by the virus. Furthermore, this prior art used intranasal immunization and/or boost to stimulate the generation of long-lasting antigen specific TRM. However, the intravenous road of administration and preparation of fresh dendritic cells is not an appropriate method for world-wide vaccination strategy. The intranasal road of administration is of interest to stimulate mucosal immunity in the long term but has been also reproducibly describe to induce allergic reaction (Vasu et al. Ther Adv Respir Dis. 2008).

Finally, the research of vaccine epitopes is a very big challenge due to the complexity of coronavirus and the huge number of epitopes that are not useful for a vaccine composition. With 30,000 genetic bases, coronaviruses have the largest genomes of all RNA viruses. Their genomes are more than three times as big as those of HIV and hepatitis C, and more than twice influenza's. 35 000 unique T-cell epitopes are of potential interest, but the final and practical clinical use will be limited to a small number of epitopes.

### SUMMARY OF THE INVENTION

The present invention provides a vaccine composition against a Severe acute respiratory syndrome-related coronavirus based on a multi-target CD8 T cell peptide composition designed for targeting several structural SARS-Cov-2 proteins such as Spike glycoprotein (S), Nucleocapsid protein (N), and Membrane glycoprotein (M) but also non-structural SARS-Cov-2 proteins, the epitopes being selected in conserved regions on the SARS-Cov-2 genome.

The vaccine composition has the following advantages:
it is pan-coronavirus and pan-CoV-2 strains;
it is design in conserved CoV-2 genome regions anticipating future mutations (up to 270 000 sequences analyzed globally to date);
the anti-viral mechanism of action is cellular, long-term and multi-targets;
it promotes generation of Tissue-resident memory sentinel T cells (Trm) in the lung and respiratory track;
it is suitable to vulnerable patients and elderly population (populations at high risk of Covid-19 morbidity and difficult to vaccinate due to immune-senescence and immune exhaustion); and
it is suitable not only for HLA-A2 positive subjects but also HLA-A2 negative subjects.

The inventors observed that a single subcutaneous injection of peptides induces a robust immunogenicity in vivo and series of epitopes induce a strong proportion of virus-specific tissue-resident memory T lymphocytes (Trm). They observed high cellular responses upon restimulation with structural and non-structural protein-derived epitopes using blood T cells isolated from convalescent asymptomatic, moderate and severe COVID-19 patients. Finally, the combination of selected CTL epitopes is suitable for use in vaccination of a broad worldwide population, even if the design was based on HLA-A2 subjects.

Accordingly, the present invention relates to a vaccine composition comprising CTL (neo)epitopes of SEQ ID NOs: 70 and/or 146; 23 and 66, and at least 2, 3, 4, 5, 6, 7 or 8 CTL (neo)epitopes selected from SEQ ID NOs: 8, 22, 31, 32, 42, 52, 77 and 97.

In one aspect, the composition further comprises at least 1 HTL peptide/epitope or a T helper peptide, especially PADRE (aKXVAAWTLKAAa with X and a respectively indicating cyclohexylalanine and d-alanine).

In a particular aspect, the vaccine composition comprises CTL (neo)epitopes of SEQ ID NOs: 8, 22, 23, 31, 32, 42, 52, 66, 70, 77, 97 and 146.

In a more specific aspect, the vaccine composition comprises CTL (neo)epitopes of SEQ ID NOs: 8, 22, 23, 31, 32, 42, 52, 66, 70, 77, 97 and 146 and a T helper peptide, especially PADRE (aKXVAAWTLKAAa with X and a respectively indicating cyclohexylalanine and d-alanine).

The vaccine composition may further comprise an adjuvant, in particular a mixture of mineral oil and mannide mono-oleate, especially Montanide® ISA 51.

In a particular aspect, the vaccine composition comprises the CTL (neo)epitopes are each at a dose of between 1 and 100 μg, preferably between 5 and 50 μg. It may comprise the T helper peptide, especially PADRE are at a dose of between 1 and 100 μg, preferably between 5 and 50 μg.

The present invention relates to a vaccine composition as disclosed herein for use for preventing or treating an infection by a severe acute respiratory syndrome-related coronavirus (SARS-CoV).

More particularly, the SARS-CoV is selected from the group consisting of SARS-CoV1, SARS-CoV2 or MERS-CoV virus, preferably SARS-CoV2.

Optionally, the subject to be treated is a subject aged 65 years or older, a subject having a cancer or having had a cancer, a subject being obese (In particular with severe obesity (body mass index [BMI] of 40 or higher [CDC-HCSP BMI>30]), a subject being diabetic, a subject having a hypertension, a subject having a sarcoidosis, a subject being immunocompromised, a subject who lives in a nursing home or long-term care facility, a subject with chronic lung disease or moderate to severe asthma, lung fibrosis, a subject who has serious heart conditions, a subject with chronic kidney disease undergoing dialysis and/or a subject with liver diseases; and/or a subject being HLA-A2.

### DESCRIPTION OF THE FIGURES

FIG. 1: T-cell epitopes location in SARS-CoV-2 genome. SARS-CoV-2 genome annotation by the Krogran lab and schematic representation of T-cell epitopes location in each encoded protein. n=4 structural proteins; n=16 non-structural proteins (NSPs); n=9 accessory factors.

FIG. 2: HLA-A2 binding characterization of WT and mutated T-cell epitopes

(A) WT and mutated peptides were incubated with HLA-A*02:01 monomer, exposed to UV for peptide exchange and then HLA-peptide complexes stability at 37° C. was measured by ELISA. Data are mean+/−SEM (n=4) expressed as percentage of binding relative to an internal MEMOPI® positive control neoepitope.

(B) WT and mutated peptides (25 μM) binding to TAP-deficient human cell line (T2) expressing HLA-A2. Data are expressed as percentage of binding relative to an internal MEMOPI® positive control neoepitope.

FIG. 3: T-cell epitope vaccination elicits broad SARS-CoV-2 protein immunogenicity in-vivo

(A) IFNγ Elispot responses of pooled spleen and draining lymph nodes cells were restimulated for 24 hours with each of the isolated peptides. Data are mean+/−SEM (n=6).

(B) Frequency of Tetramer+CD8 T cells (gating strategy in FIG. 4A). Medium and High response threshold were defined based on 2-fold or 3-fold increase respectively compared to the background frequency measured in the non-vaccinated mice control group. Controls+ are Memopi® peptides with previously validated immunogenicity. Data are mean+/−SD of pooled female (n=3) and pooled male (n=3) vaccinated mice.

FIG. 4: T-cell epitope vaccination elicits Tissue-resident memory viral-specific CD8 T cells

(A) Flow cytometry gating strategy to define Tetramer+CD8 T cells responses.

(B) Representative example and gating strategy of Trm phenotype based on CD44, CD103, CXCR3, CD49a and CD69 expression in Tetramer+CD8 T cells as compared to Tetramer−CD8 T cells.

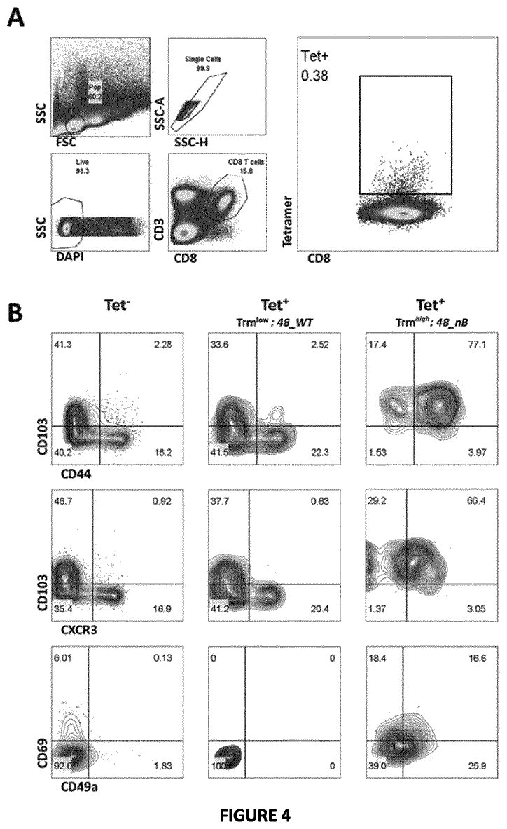

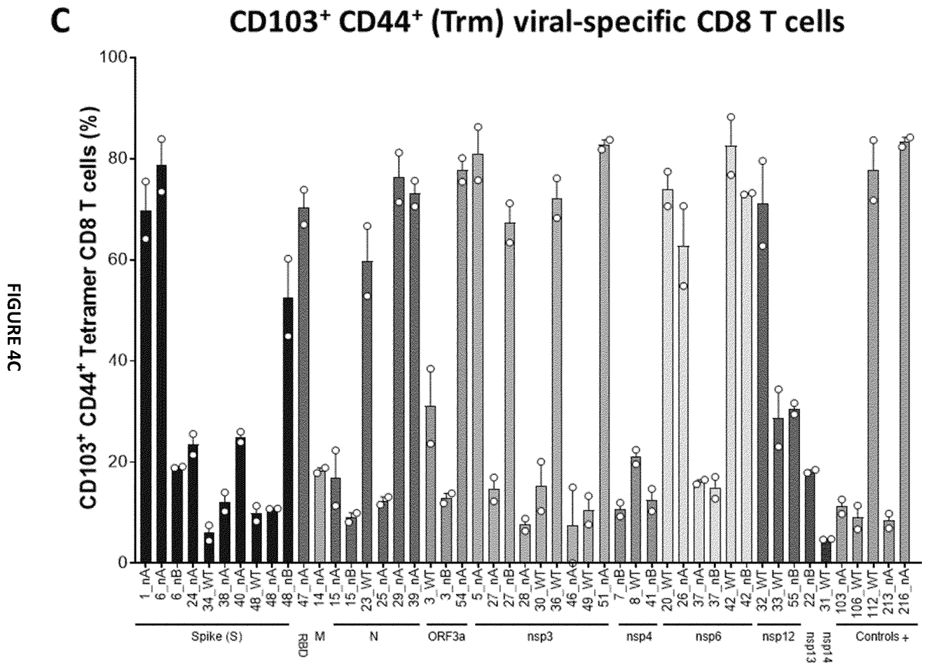

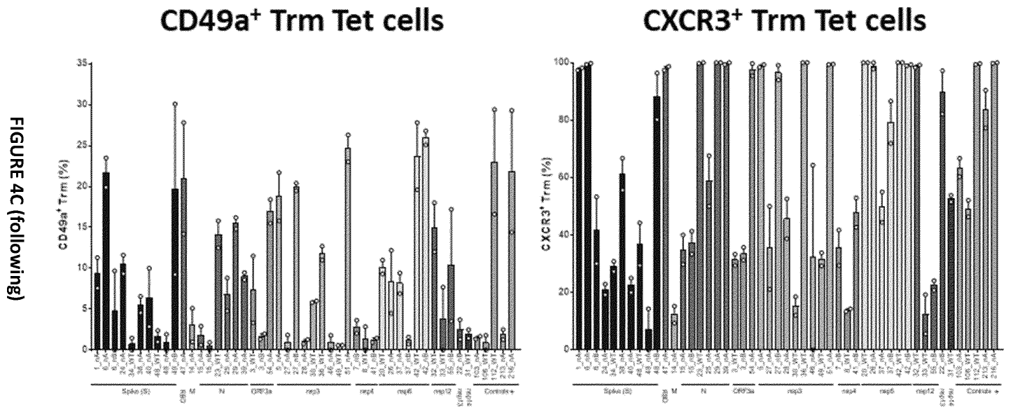

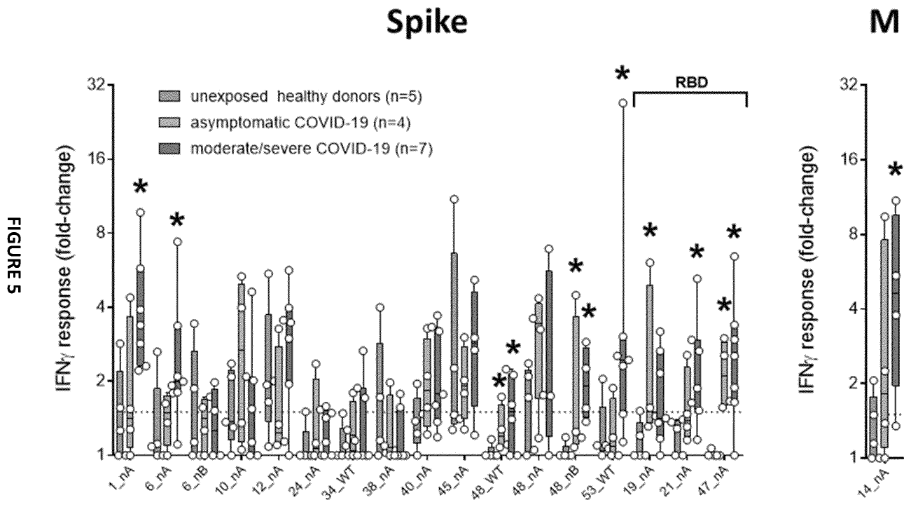

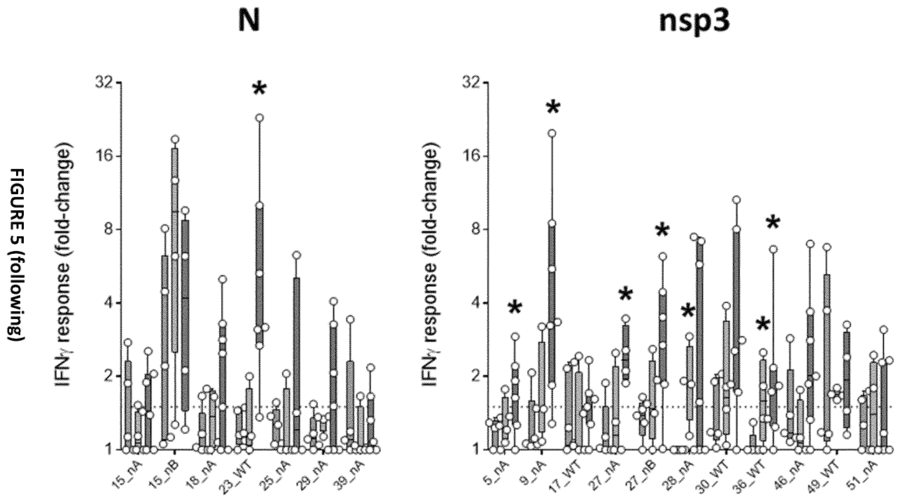

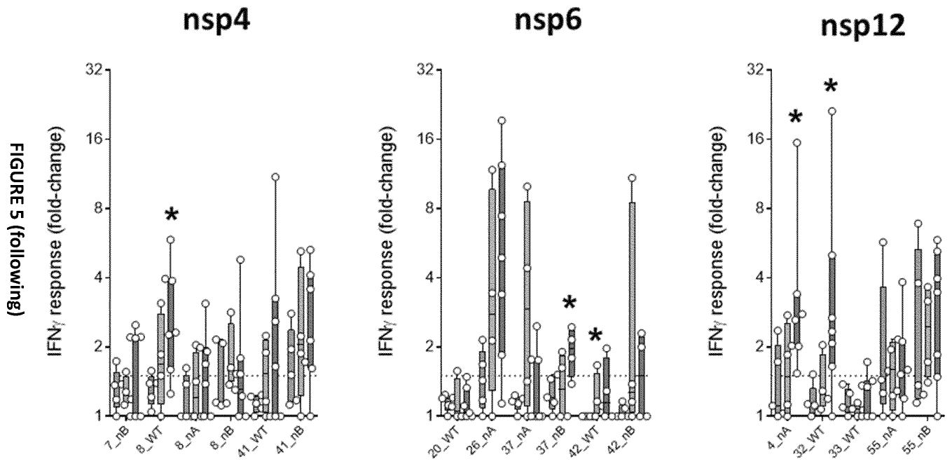

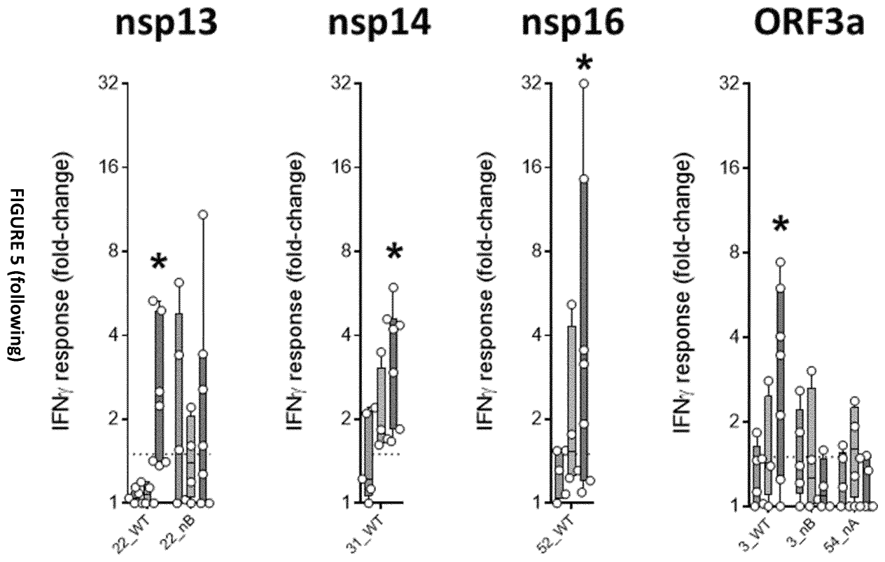

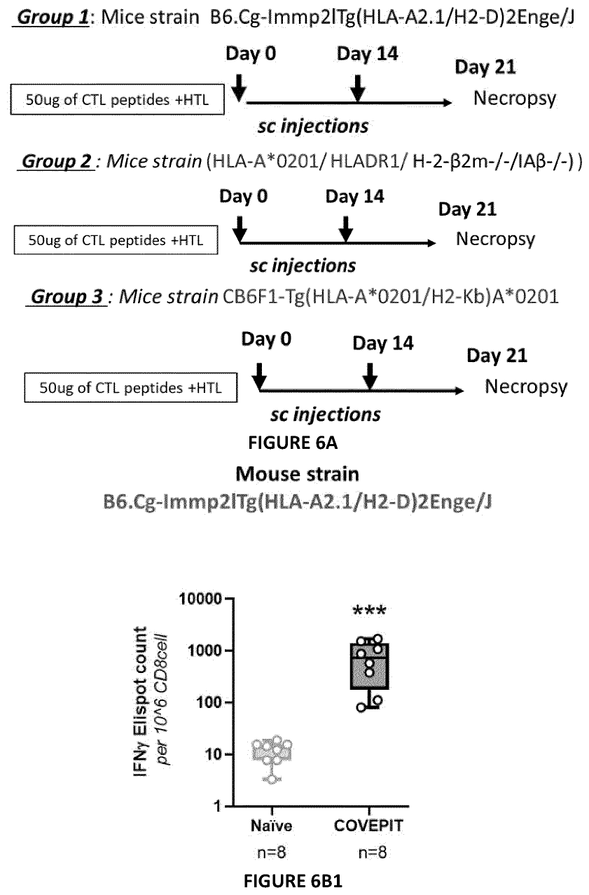

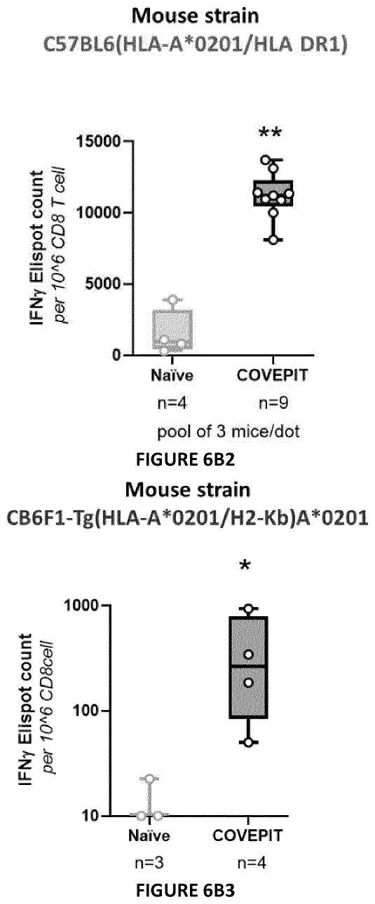

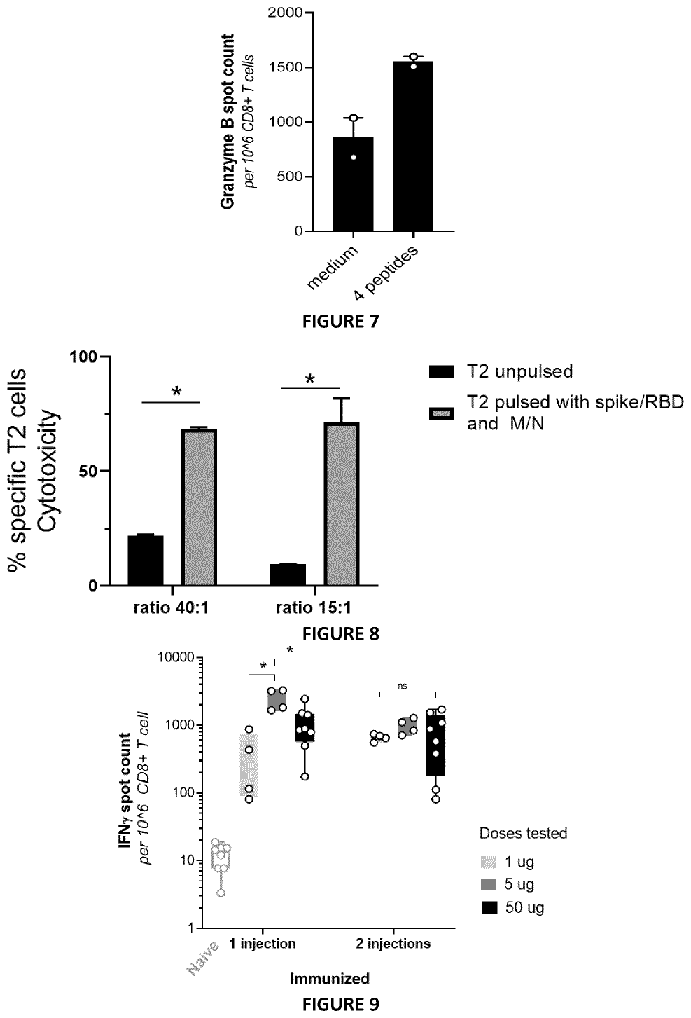

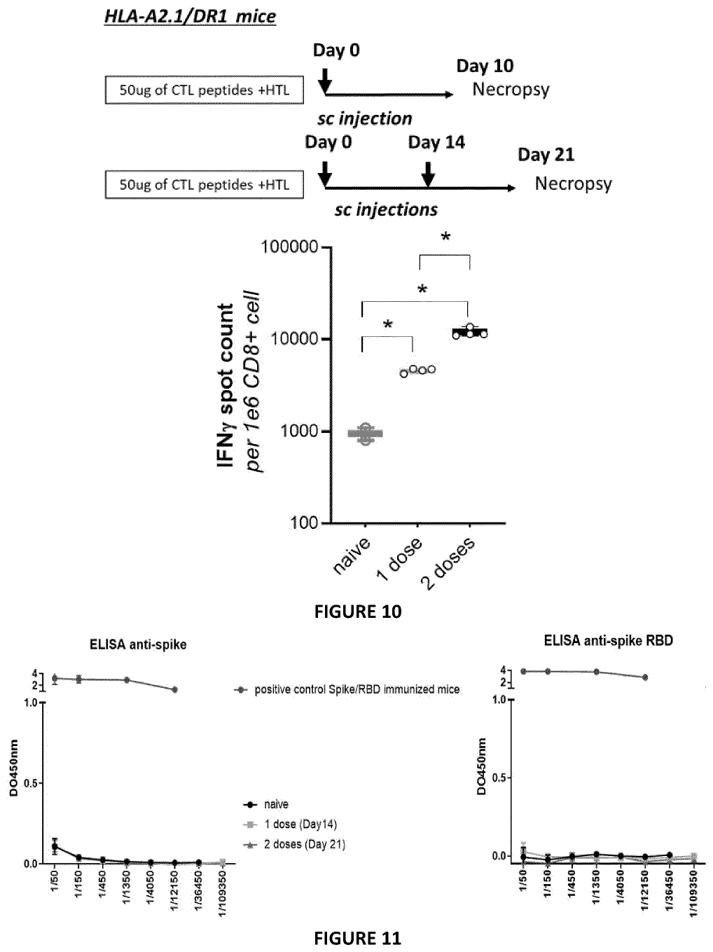

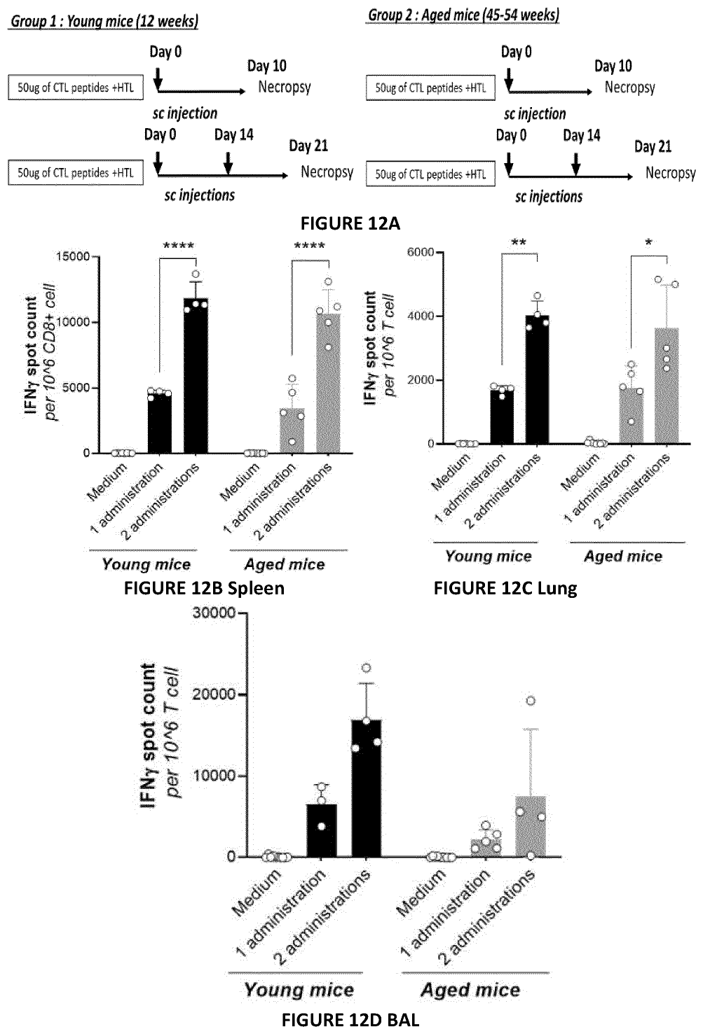

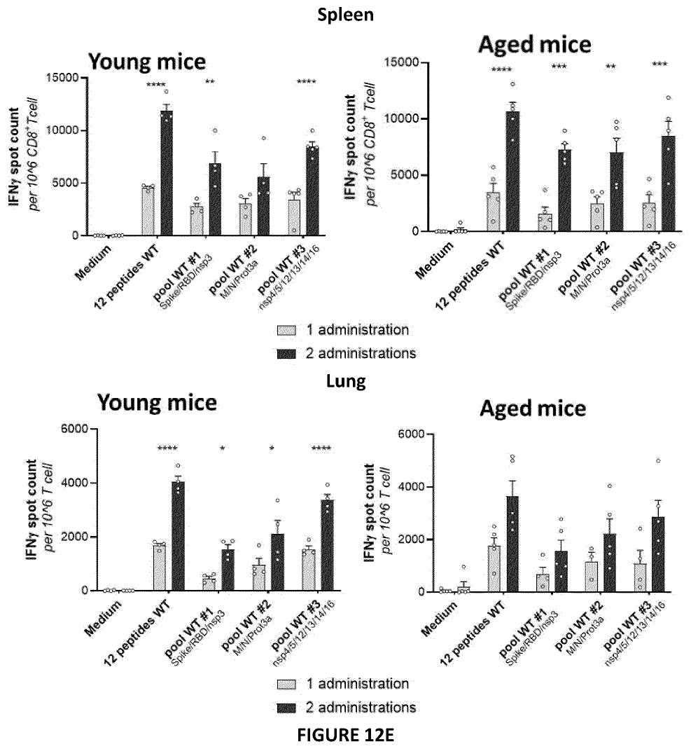

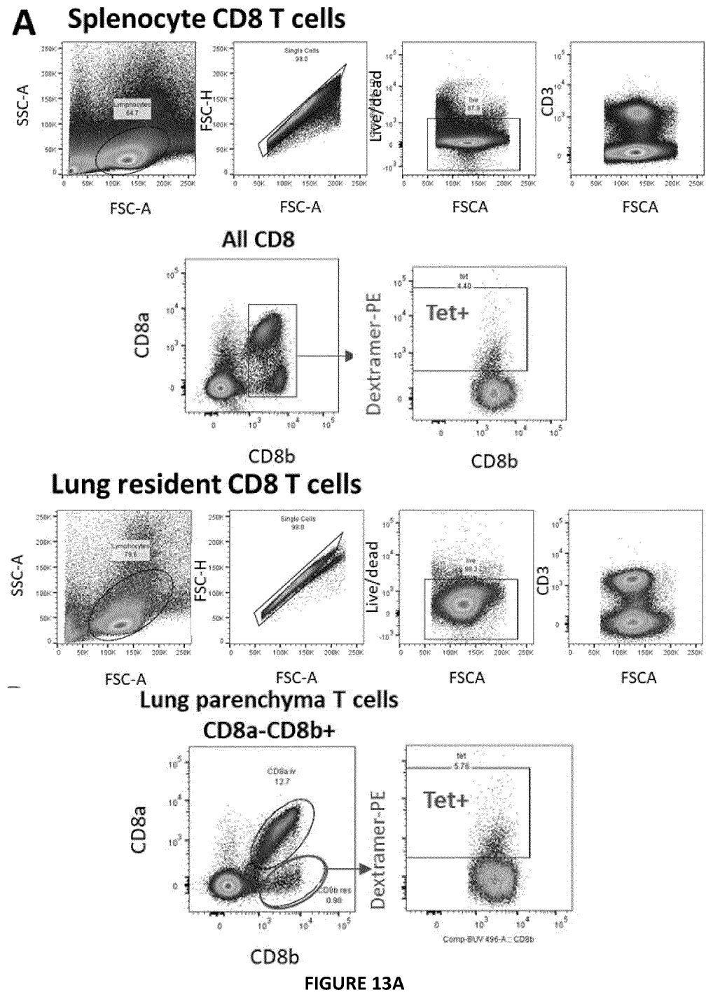

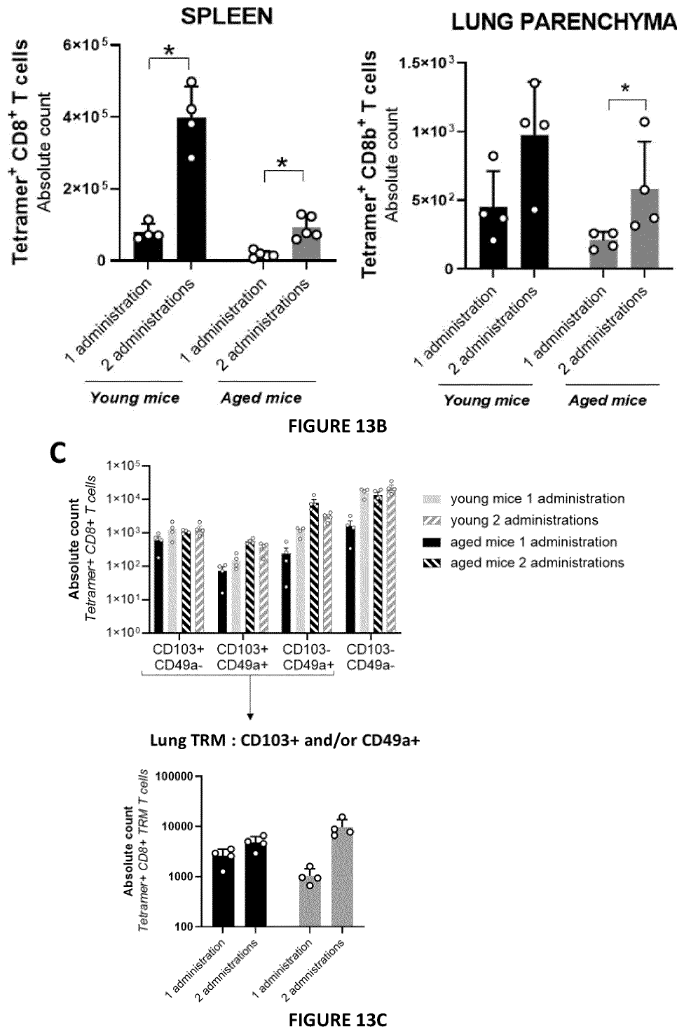

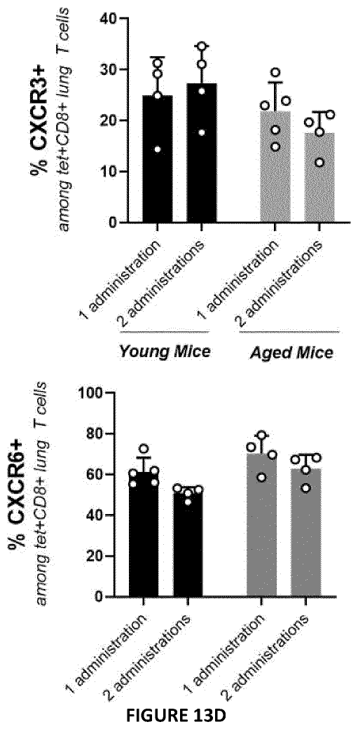

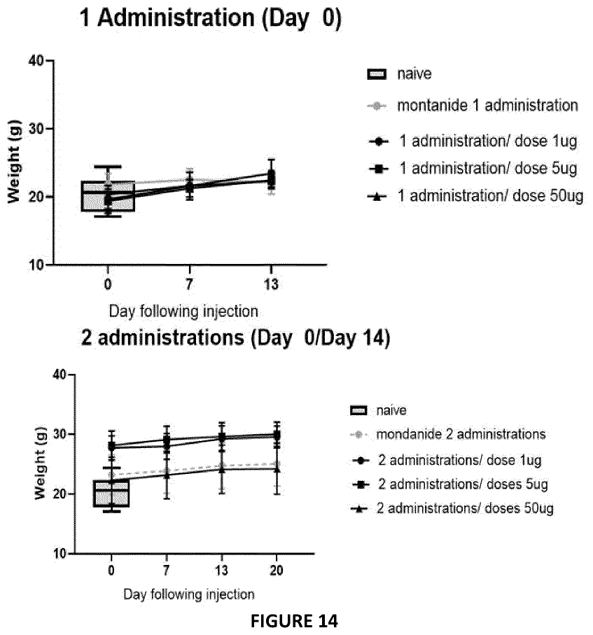

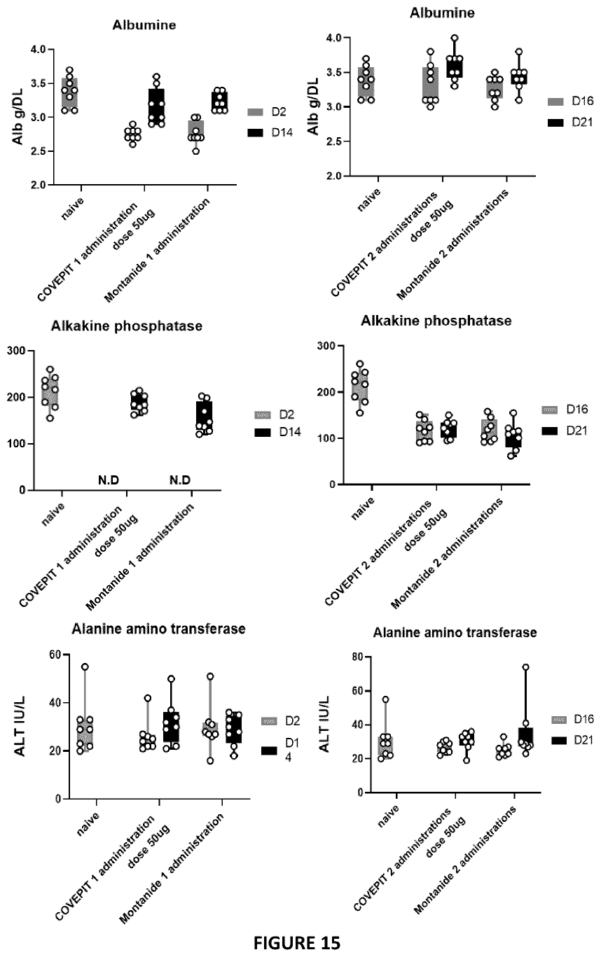

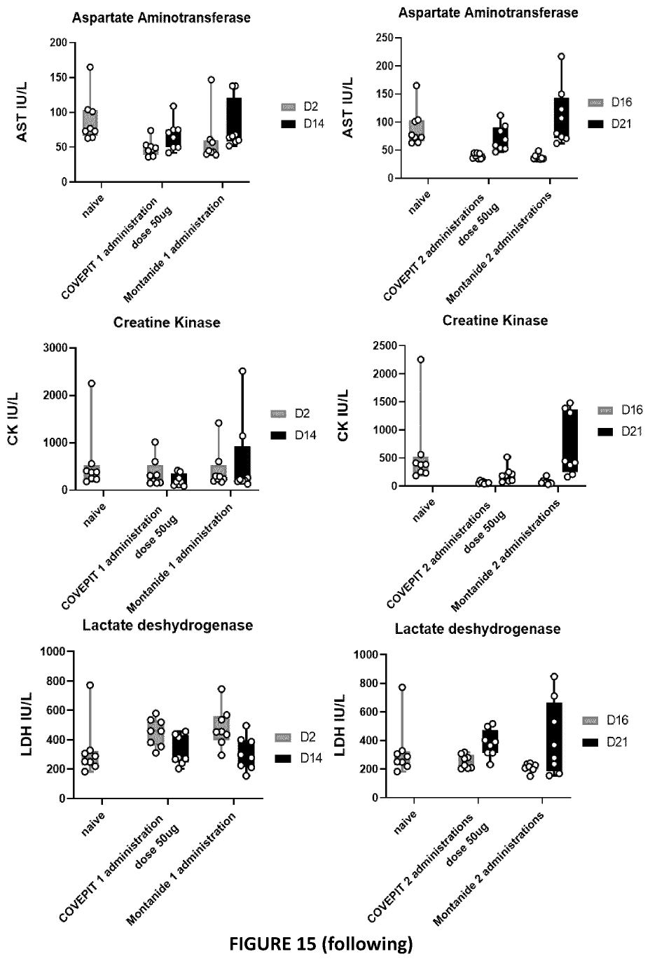

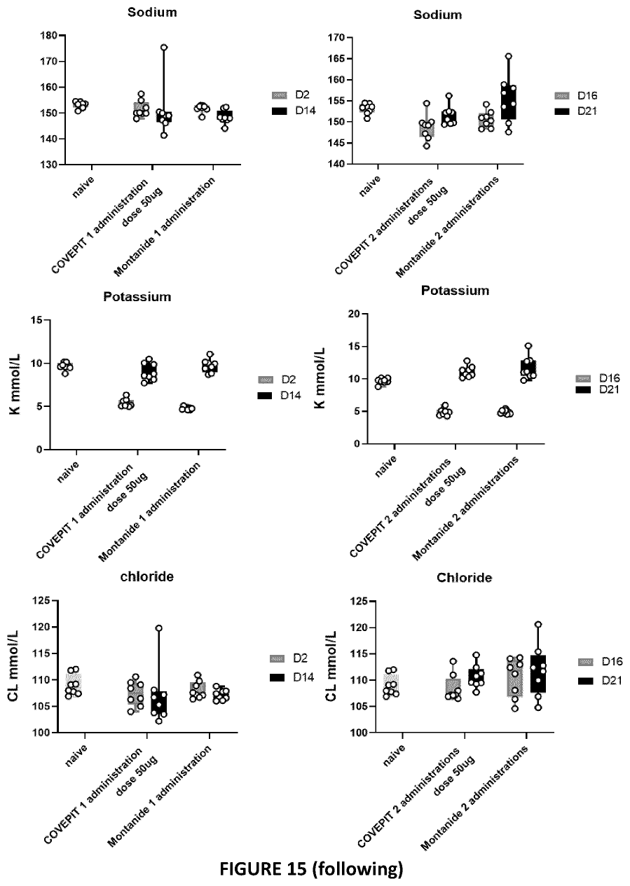

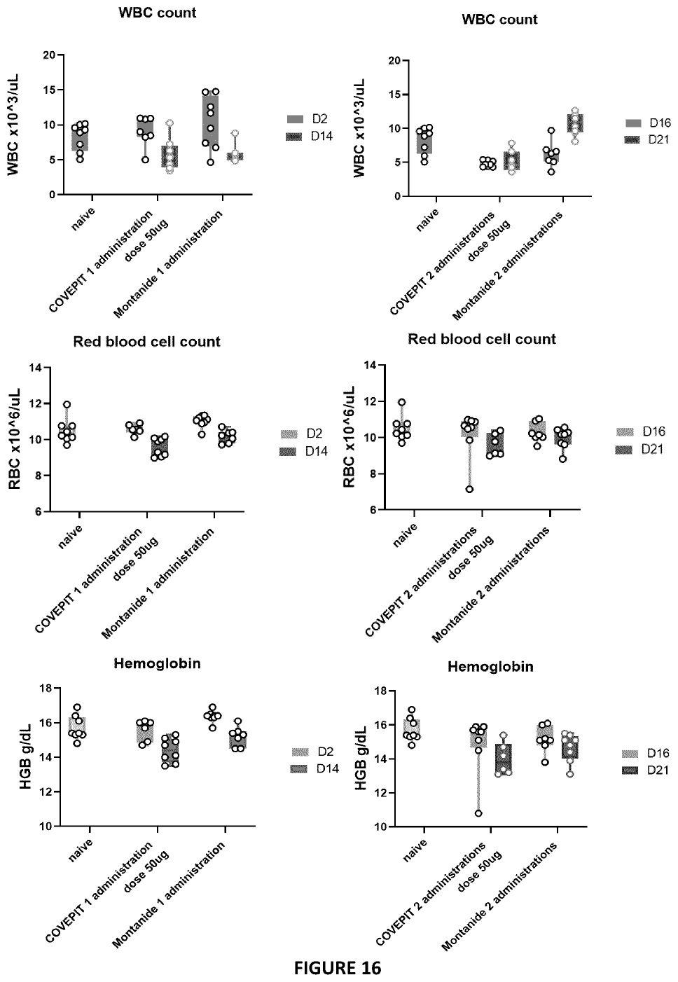

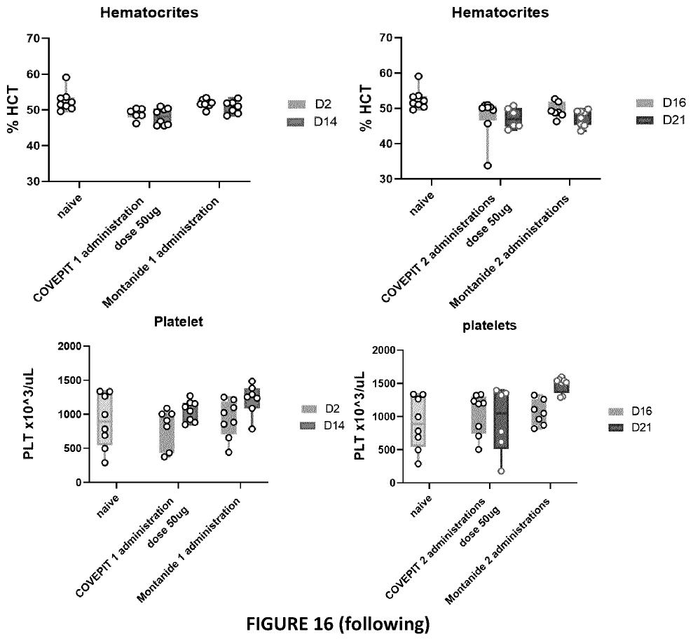

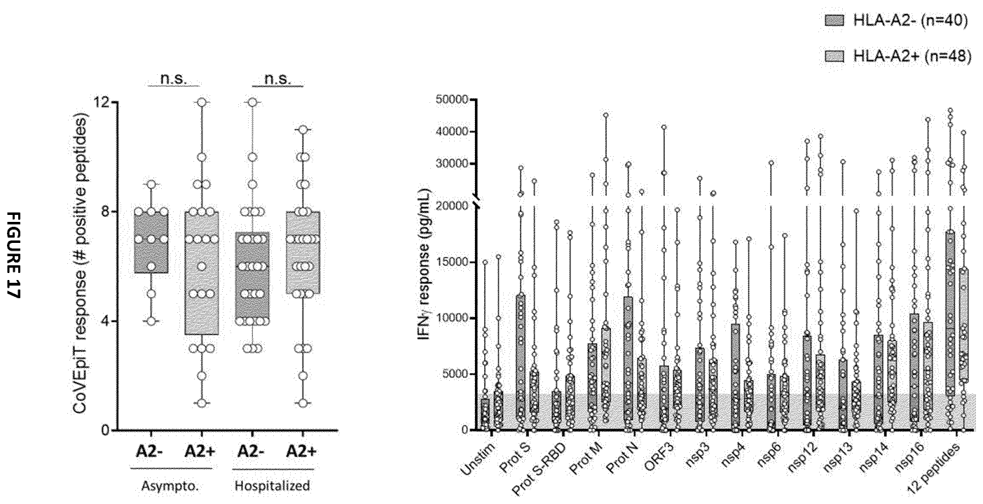

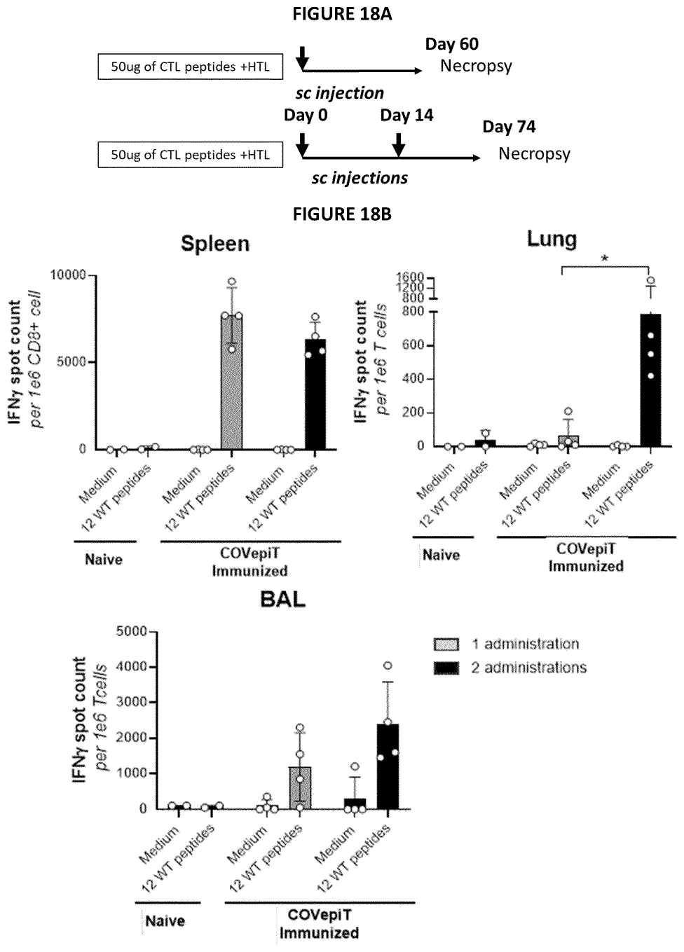

(C) FIG. 4C: Frequency of CD103+CD44+(defined as Trm) cells within Tetramer+CD8 T cells (defined as medium or high response based on FIG. 3). FIG. 4C following: Frequency of CD49a+ or CXCR3+ cells within Trm+Tetramer+CD8 T cells. Controls+ are Memopi® peptides with previously validated immunogenicity. Data are mean+/−SD of pooled female (n=3) and pooled male (n=3) vaccinated mice.

FIG. 5: Ex-vivo T-cell epitope reactivity in asymptomatic and convalescent COVID-19 individuals and unexposed donors. IFNγ secretion responses for 48 hours after one week of restimulation of human PBMC from unexposed HLA-A2+ healthy donors (n=5), asymptomatic confirmed COVID-19 HLA-A2+ individuals (n=4) and moderate or severe COVID-19 HLA-A2+ convalescent patients (n=7) with each of the isolated peptides and HLA-A2+ antigen-presenting cells. Data were normalized with negative control peptides. Data are expressed as mean+/−min to max. *p<0.05

FIG. 6: CoVepiT elicits a strong CD8 T cell immunogenicity in vivo in different HLA-A2+ transgenic mice.

FIG. 6A. Experimental design of CoVepiT immunization. Three different strains of mice were tested: (group 1) a HLAA2+ model only; (group 2) a HLAA2+/HLA-DR1+ double transgenic mice (Pasteur stain); (group 3) a HLAA2+ model only (Taconic strain). 50 μg of each peptide+HTL peptide emulsified with Montanide were injected twice. FIG. 6B. IFN-γ response was measured by Elispot after in vitro restimulation with 12 wild-type peptides and calculated for 10⁶ CD8 T cells. FIG. 6B1: 0.3×10⁶ CD8+ cells were stimulated. FIG. 6B2 and FIG. 6B3: 10⁵ CD8+ T cells cocultured with 10⁵ CD8− cells for stimulation. *p=0.03; ***p=0.0002 and **p=0.003 calculated with Mann-Whitney statistical test.

FIG. 7: Granzyme B secretion of CTL specific T cells after immunization. Granzyme B Elispot assay after ex vivo restimulation with 4 viral peptides Spike/Receptor Binding Domain/Membrane/Nucleocapsid (S/RBD/M/N) of CD8 T cells isolated from immunized mice. Each dot represents a pool of 3-4 mice and Histogram represents mean+/−SEM.

FIG. 8: In vitro killing activity of CTL specific T cells against SARS-COV2 pulsed target cells mimicking infected cells. Chromium 51 release assay performed with unpulsed T2 cells (negative control cells without viral peptides presentation at the surface) or peptides pulsed (by wild-type S/RBD+ M/N SARS-COV2) presented at the surface of T2 cells as target cells. CD8 effector T cells isolated from immunized CoVepiT mice can kill the T2 cells mimicking infected cells. This killing or the cytotoxicity activity of the CD8+ T cells was measured through the Cr51 release and was quantified following 4 hours of coculture at 2 different Effector: Target ratios. Data are mean+/−SEM of triplicata of pooled immunized mice. Statistical significance was calculated with unpaired t test *p<0.05.

FIG. 9: CoVepiT Dose escalation—one or two administration in HLA-A2 transgenic mice. Three escalating doses were studied 1 μg n=4; 5 μg n=4, 50 μg n=8 versus naïve mice n=8. One or two administrations of the same dose of CoVepiT were injected subcutaneously into mice. IFN-γ response was measured by Elispot after in vitro restimulation (0.3×10⁶ CD8 T cells) with 12 wild-type peptides for 24 hours. After one injection the dose of 5 μg of CoVepiT gives significantly the best IFNg response versus the naïve group and the group 1 μg and 50 μg. After two injections, no significant difference was observed in immunogenicity in this model between one or two administrations and between the 3 doses 1, 5 or 50 μg tested.

FIG. 10: One administration versus two administrations assessment in HLA-A2/DR1 transgenic mice. One or two administrations of CoVepiT were injected subcutaneously (12 weeks old HLA-A2/DR1 mice) 24 mice were administrated with 50 μg/CTL peptide+HTL comparing 1 or 2 injections D0 or D0+D14). Naïve mice (n=6) were used as control. IFN-γ response was measured by Elispot after in vitro restimulation for 24 hours of 5·10⁴ CD8+ T cells with 5·10⁴ CD8− cells pulsed with 12 SARS cov2 wild-type peptides (10 μg/m L) each. Each dot represents a pool of 3 mice. Statistical significance was calculated with one-way ANOVA for multiple comparison. *p<0.05.

FIG. 11: CoVepiT vaccine does not elicit antibody response against Spike and RBD proteins. On Day 14 (1 administration of 50 μg of each peptide+25 μg HTL) or on Day 21 (2 administrations of 50 μg each peptide+25 μg HTL) following CoVepiT immunization, sera were collected. Anti-Spike or anti-RBD specific ELISA was performed by immobilizing spike (S1+S2) and RBD proteins on the ELISA plate (2 μg/mL). Serum of immunized mice was serially diluted then added to the plate. Anti-mouse IgG and IgA antibodies were detected with a peroxidase goat polyclonal anti-mouse IgG+IgA (1 μg/mL). Revelation was assessed with Tetramethylbenzidine substrate and colorimetry quantified at DO 450 nm. Data represent mean+/−SD of the DO after subtraction with negative control (blank no serum). N=8 mice/group (total n=24 mice). Positive control is a serum of Spike/RBD immunized mice containing specific anti spike and RBD IgG and IgA antibodies.

FIG. 12: CTL Immunogenicity of CoVepiT vaccine in young and old HLA transgenic mice. FIG. 12A. Experimental design Twenty-one young mice and thirty aged mice, dose of 50 μg of each peptide administered Once D0 (necropsy at D10) or twice DO D14 (necropsy at D21). FIG. 12B. IFN-γ Elispot analysis of splenic isolated CD8+ T cells and ratio 1:1 (5×10⁴ CD8+: 5×10⁴ CD8−) after restimulation with pool of 12 wild-type CTL epitopes or media (negative control). FIG. 12C. IFN-γ Elispot analysis of lung isolated T (1×10⁵ T cells) and CD8− cells (5×10⁴ T cells) after restimulation with pool of 12 wild-type CTL epitopes or media (negative control). FIG. 12D. IFN-γ Elispot analysis of BAL isolated T cells (2×10⁴) and CD8− cells (5×10⁴ T cells) after restimulation with pool of 12 wild-type CTL epitopes or media (negative control). FIG. 12E. IFN-γ Elispot analysis of spleen and lung after stimulation with media pool of 12 pool peptides or pool peptide #1 Spike/RBD/nsp3 (19/27/48), pool #2 M/N/ORf3 (14/23/54), pool #3 nsp 4/5/12/13/14/16 (8/22/31/32/42/52) in the same condition as described in B and C. All wild-type corresponding peptides were used for stimulation. Each dot represents a pool of 3 mice, Histogram represent mean+/−SD of IFNg spot Count calculated for 10⁶ CD8+ T cells and statistical significance was calculated with One-way Anova for multiple comparisons, *p<0.04; **p<0.004, ***p<0.0003 ****p<0.0001.

FIG. 13: CoVepiT vaccination elicits CTL viral-specific resident memory T cells into the lung. FIG. 13A. Flow cytometry gating strategy for tetramer+ viral specific T cells. Twenty-four young mice and thirty aged mice dose of 50 μg administered Once D0 (necropsy at D10) or twice DO D14 (necropsy at D21) Tetramer+(tet+) was quantified into CD8αβ+ splenic T cells and CD8α−β+ resident memory T cells. CoVepiT epitopes loaded on PE labeled HLA-A2.1 Dextramer were used for the staining. FIG. 13B. Tetramer+ total absolute count per spleen or lung in young versus aged mice. FIG. 13C. Absolute count of Tet+ Trm CD8 T cells into the lung of immunized mice. Trm phenotype was based on expression of at least CD103 or CD49a marker. FIG. 13D. Frequency of CXCR3 and CXCR6 in tet+CD8+ lung T cells. Each dot represents a pool of 3 mice, Histograms represent mean+/−SD.

FIG. 14: Mouse weight measurement after 1 injection or 2 injections of CoVepiT. Top: One injection: in pharmaco-toxicology part: comparisons of the group CoVepiT receiving the highest dose 50 μg versus the naïve mice group (not treated) and the group of Montanide adjuvant injected Once. Down: two injections DO D14: in pharmaco-toxicology part, comparison of the group CoVepiT receiving the highest dose at 50 μg versus the group of Montanide adjuvant injected twice. Represented in the figure for information, the 2 lower other doses of CoVepiT tested in other groups (1 μg and 5 μg) to measure the immunogenicity of CoVepiT and a potential dose effect with 3 increasing doses.

FIG. 15: Clinical chemistry parameters following vaccination after one or two injections. Blood Biological measures (albumin, Alkaline phosphatase, AST, ALT, CK, LDH, Sodium, Potassium, Chloride) were measured after CoVepiT test item administered at 50 μg (at D2 D15 after one injection) versus the naïve mice timepoint at D0 (not treated mice) or versus the Montanide group at the same timepoints (D2 D14 after one injection). The test item was also administered twice with measure at D16 D21 after two injections and compared with Montanide groups at the same timepoints (at D16 D21 after two injections). N.D. Not dosed.

FIG. 16: biological and blood cell counts following One or Two vaccinations. White blood cell count; Red Blood cell; Hemoglobulin; hematocrit; platelets, all items were measured after CoVepiT test item administered at 50 μg (at D2 D14 after one injection) versus the naïve mice timepoint at DO (not treated mice) or versus the Montanide group—one injection—at the same timepoints (D2 D14 after one injection). The test item CoVepiT was also administered twice with measure at D16 D21 after two injections and compared with Montanide group—Two injections—at the same timepoints (D16 D21). N=6-8 mice per group, Blood of 1 or 2 mice per group could not be analyzed due to the formation of clot at the time of collection. Statistical significance was calculated with one-way Anova followed by Tukey's multiple comparisons. test. ** P≤0.007. *** P≤0.0005 **** P<0.0001; ns for non-significant. No significant differences observed in the blood cell counts after one injection. No significant difference observed after 2 injections of CoVepiT, except a slight increase in monocyte % and a slight decrease in lymphocytes % after 2 injections of CoVepiT versus naïve and montanide groups receiving 2 injections.

FIG. 17: Immunogenicity in all COVID-19 Patients and large HLA coverage.

Left. Same level of responses observed in HLA-A2 positive patients (green color (n=48)) versus HLA-A2 negative patients (orange color (n=40)), that patients being asymptomatic or hospitalized.

Right. The immunogenicity was measured in the IFN gamma responses after stimulation with the peptides using blood T cells isolated from HLA-A2 positive COVID-19 patients (n=48) and HLA-A2 negative COVID-19 patients (n=40).

FIG. 18: CoVepiT vaccination elicits long-term CTL viral-specific memory T cells response. A. Experimental design. One or two administrations of CoVepiT were injected subcutaneously (HHD mice HLAA2 transgenic mice); 12 mice received one injection (Day 0), 12 mice received 2 injections (Day 0 and day 14) and 6 mice were not immunized (Naïve). Mice were sacrificed on Day 60 or Day 74 to measure IFN-γ response. B. IFN-γ response was measured by Elispot after in vitro restimulation for 24 hours with Medium (negative control) or with 12 SARS cov2 wild-type peptides (10 ug/mL) each. For the spleen, CD8+ T cells (5×10⁴ cells) were stimulated with CD8− cells (ratio 1:1). For the lung and BAL, isolated T cells were stimulated. Each dot represents a pool of 3 mice. Statistical significance was calculated with one-way ANOVA followed by Tukey's multiple comparison test. *p<0.05.

### DESCRIPTION OF THE INVENTION

The vaccine presented in the invention assembles (neo)epitopes combination assuring the involvement of the full repertoire of cells involved in the immune responses to this Specific SARS Coy infection.

The immune cells activated by the vaccine could be in particular:
Cytotoxic T Lymphocytes (designated CTL) that are capable to destroy cells that are infected by the virus;
Helper T Lymphocytes (HTL) that are capable to activate both B and T responses to induce long-term memory immune response; and
B cells Lymphocytes (BCL) that produce neutralizing antibodies.

In a preferred aspect, the selection of the epitopes/neoepitopes of the vaccine composition allows to provide early B cell & HTL response and/or long T cell memory CTL and HTL responses.

This strategy provides advantageously a vaccine composition activating:
the short term (notably prophylactic) response of antibodies; and
the long term (notably prophylactic) response through the activation of CTL and also eventually of HTL.

The activation of T cells response is of major interest for coronavirus which particularly induces a major problem of T cells response. Further, since some T cells in particular memory T cells are specifically localized in pulmonary tissue, their activation is very helpful against coronavirus which has severe consequence on pulmonary tissue.

In preferred aspect, the vaccine comprises epitopes of the coronavirus that will be recognized by CD8 T cells through the interaction with MHCI system. The activated CD8 T cells convert into Lymphocytes cytotoxic T cells (effector CTL) by the help of helper T Lymphocytes (HTL). The activated T cells are notably CD8 memory T cells allowing the long-term action of the vaccine.

The process of selection of epitopes and their modifications into neoepitopes is a key element of the invention. A second element is the epitopes/neoepitopes combination able to produce a synergy of the immune responses. A third element is the generation of (neo)epitopes with high homology between coronavirus such SARS-CoV (2003), MERS-CoV (2012) and SARS-CoV-2 (2019) allowing vaccination against several and future emergent coronavirus.

As reminded above, a very high number of ^{˜}35 000 unique T-cell epitopes in SARS-CoV-2 genome could be identified. About 200 epitopes could be selected based on the binding capacity by HLA-A2 and their immunogenicity.

Thanks to further complex studies, the inventors further refined the selection by the identification of 55 very promising CTL epitopes of interest (as disclosed in Table 1). The selected epitopes are issued from 4 different parts of the coronavirus (Spike protein (S) (including RBM as epitope B), Membrane protein (M), Nucleocapside (N), and several non-structural viral proteins from viral RNA).

The selected epitopes have the advantages to be well-conserved among SARS-CoV coronaviruses, in particular SARS-CoV-2, SARS-CoV1 and MERS-CoV genomes.

In addition, from the 55 very promising CTL epitopes, the inventors further designed neo-epitopes with an increased binding and/or immunogenicity. These neo-epitopes of interest are disclosed in Tables 2 and 3.

Among these CTL epitopes, the inventors selected a group of 46 preferred CTL (neo)epitopes which induce an immune response in vivo (see Table 7); a group of 27 preferred CTL (neo)epitopes which induce an immune response in vivo and induce cellular responses upon restimulation with these CTL (neo)epitopes using blood T cells isolated from convalescent asymptomatic, moderate and severe COVID-19 patients (see Table 8). Among these peptides, some induce a strong proportion of virus-specific tissue-resident memory T lymphocytes (Trm) (see Tables 7 and 8). Based on these data, the inventors provide a list of preferred CTL peptides (see Table 9). Based on these 12 CTL peptides, it is possible to induce a vaccination of a broad worldwide population (see Table 10). Peptides are able to bind several HLA and show a worldwide population coverage in the range of 40-99%. As shown in Table 10, it is possible to obtain at least one immune response in everybody and to reach herd immunity (65%) in the worldwide population with at least one combination of 5-7 CTL peptides.

Finally, the inventors designed a group of epitopes suitable for inducing an immune response by B Lymphocytes (BCL) that produce neutralizing antibodies, therefore called herein BCL epitopes. B epitopes are selected in the precise 420-500 region of the Spike protein in order to create or generate antibodies blocking the entry of the virus, through an antagonist action of the antibody blocking the recognition between the virus epitopes and the cells of the host. B cell epitopes were rationally design selectively in the receptor-binding domain (RBD), more particularly within the receptor-binding motif (RBM), of the protein Spike. In contrast to other vaccinal strategies with live, attenuated or inactivated virus expressing the whole Spike proteins or vaccination with DNA or RNA coding for Spike or RBD, or vaccination with the total Spike proteins, here by designing selectively B-cell epitopes within the RBM motifs, there is not risk to generate non-neutralizing antibodies which have been associated with antibody-dependent enhancement phenomenon (Iwasaki et al. Nature Review Immunology 2020).

These BCL epitopes of interest are disclosed in Table 4. In addition, these BCL epitopes can be fused to HTL epitopes. For instance, HTL epitopes can be PADRE and such fused epitopes are disclosed in Table 5. The BCL epitopes could be directly coupled or covalently linked to an HTL epitope with an adaptor or through a linker.

The vaccine according to the present invention is a multi-(neo)epitopes combination (wildtype and neoepitopes) with HTL, BCL or CTL purpose so as to induce a synergistic immune response.

The synergy provided by the final combination is a strong element of the invention supporting the original concept of this versatile strategy adapted to the SARS-Cov vaccination in order to obtain an adequate robust response and to limit doses of vaccine.
Epitope and neoepitopes confers synergy as the quantity of each peptides could be limited for the same immune response levels versus a vaccine limited to a few numbers of epitopes (one or two epitopes)
HTL favorizes both BCL responses and TCL responses as CD4+ T cells interact directly with antigen presenting cells to enhance their ability to present viral antigen to T cells
BCL− HTL combination favorizes the effects of TCL− HTL combination
BCL− HTL combination boosts combination TCL− HTL combination.

The final selection for the vaccine composition is based on clinical needs (early responses, long term responses) depending of the state of the patients, immediate risk or long-term risks, evolution of the pandemic and the general condition of the patients (fragile or aged, immunocompromised, debilitating conditions patients).

Although neutralizing antibody production by B cells and cytotoxic activity of CD8+ T cells are well-accepted components of the adaptive immune response to various viruses, the role of the two segments of immunity is rarely covered in one specific vaccine strategy. To consider the COVID-19 pandemic situation and the resurgence of the virus in seasonal condition, it will be important to anticipate specific clinical situation with the ability to adapt the combination to the situation.

In the previous infection to the first SARS-Cov, the frequency of CD4+ memory T-cells to virus structural proteins and anti-SARS coronavirus IgG levels were low by 12 months after infection (Libraty H T et al; Science direct Virology 2007—Human CD4+ memory T-lymphocyte responses to SARS coronavirus infection).

Favorizing early B cell responses and IgG neutralizing antibody for pandemic acute periods is a clinical need for a wide population considered at risks of infection.

Favorizing T cells long term responses for specific populations with a serious risk of mortality is another clinical need covered by the invention.

The vaccine composition has also been designed in order to be effective, not only on the known SARS-Cov viruses but also against SRAS-Cov may emerge in the future.

This whole work leads to a selection of combinations of for instance 10 to 20 epitopes/neoepitopes, fighting against the heterogeneity of the previous, current and future coronaviruses. The selection of the epitopes/neoepitopes can be based on various possible modalities, notably:
a combination of BCL, CTL and HTL epitopes and neoepitopes;
a combination of BCL and CTL epitopes and neoepitopes;
sequentially administered first a combination of BCL and HCL epitopes and then a combination of CTL and HCL epitopes and neoepitopes (or the inverse order);
sequentially administered first a combination of BCL epitopes and then a combination of CTL epitopes and neoepitopes (or the inverse order)
a combination of BCL and HTL epitopes; or
a combination of CTL and HTL epitopes and neoepitopes.

In summary the invention describes a (neo)epitopes—based vaccine selected on high binding capability or by specific chemical modification increasing binding property, addressing early (HTL and B cell specific immune response) and/or long-term immunogenicity (HTL and T cells specific immune response) in particular for “fragile” patients.

According to a first aspect, the vaccine composition comprises:
At least 1, preferably at least 5, notably 5 to 15 or 5 to 20, notably 2, 3, 4, 5, 6, 7, 8, 9, 10, 11, 12, 13, 14, or 15 peptides CTL epitopes or neo-epitopes, for activating CD8 CTL cells;
At least 1, preferably at least 2, notably 2 to 8, notably 2, 3, 4, 5, 6, 7, or 8 peptides BCL epitopes inducing specific antibodies production; and
Optionally at least 1 epitope targeting HTL cells capable of inducing both T response and B response, for instance 1, 2, 3, 4 or 5 HTL epitopes.

Then, the CTL epitopes and the BCL epitopes of the vaccine are mixed in a common vaccine composition that is administered in one injection, repeated if appropriate.

According to a second aspect, the vaccine composition comprises:
At least 1, preferably at least 5, notably 5 to 15 or 5 to 20, notably 2, 3, 4, 5, 6, 7, 8, 9, 10, 11, 12, 13, 14, or 15 peptides CTL epitopes or neo-epitopes, for activating CD8 CTL cells; and
Optionally at least 1 epitope targeting HTL cells capable of inducing both T response and B response, for instance 1, 2, 3, 4 or 5 HTL epitopes.

According to a third aspect, the vaccine composition comprises:
At least 1, preferably at least 2, notably 2 to 8, notably 2, 3, 4, 5, 6, 7, or 8 peptides BCL epitopes inducing specific antibodies production; and
Optionally at least 1 epitope targeting HTL cells capable of inducing both T response and B response, for instance 1, 2, 3, 4 or 5 HTL epitopes.

According to a fourth aspect, the invention relates to a combination of two vaccine compositions, the first composition comprising:
At least 1, preferably at least 5, notably 5 to 15 or 5 to 20, notably 2, 3, 4, 5, 6, 7, 8, 9, 10, 11, 12, 13, 14, or 15 peptides CTL epitopes or neo-epitopes, for activating CD8 CTL cells; and
Optionally at least 1 epitope targeting HTL cells capable of inducing both T response and B response, for instance 1, 2, 3, 4 or 5 HTL epitopes.

And the second composition comprising:
At least 1, preferably at least 2, notably 2 to 8, notably 2, 3, 4, 5, 6, 7, or 8 peptides BCL epitopes inducing specific antibodies production; and
Optionally at least 1 epitope targeting HTL cells capable of inducing both T response and B response, for instance 1, 2, 3, 4 or 5 HTL epitopes.

Then, the CTL epitopes and the BCL epitopes of the vaccine are in two separate compositions, that are administered sequentially, firstly a CTL epitopes composition then BCL epitopes composition, or firstly BCL epitopes composition then CTL epitopes composition, repeated if appropriate.

Optionally, the BCL epitopes can be fused to the HTL epitopes.

In a preferred aspect, the total number of epitope peptides in the composition can be from 5 to 40, from 7 to 30 or from 10 to 20 peptides.

In a preferred aspect, the CTL epitopes are a mixture of naïve T epitopes (CTL epitopes) and of neo-epitopes (CTL neo-epitopes), advantageously 1 to 15 CTL epitopes and 1 to 15 CTL neo-epitopes. The CTL epitopes are selected from the CTL epitopes of Table 1 and the CTL neo-epitopes are selected from the CTL neo-epitopes of Tables 2 and 3, of Table 2 or of Table 3.

In another preferred aspect, the CTL epitopes are a mixture of naïve T epitopes (CTL epitopes) and of neo-epitopes (CTL neo-epitopes), advantageously 1 to 15 CTL epitopes and 1 to 15 CTL neo-epitopes. The CTL epitopes are selected from the CTL epitopes of SEQ ID NOs: 3, 8, 20, 22, 23, 30, 31, 32, 33, 34, 36, 42, 48, 49 and 52 and the CTL neo-epitopes are selected from the CTL neo-epitopes of SEQ ID NOs: 56, 59, 60, 66, 67, 70, 74, 75, 76, 77, 78, 79, 83, 84, 85, 86, 90, 91, 92, 95, 97, 101, 104, 105, 113, 120, 125, 135, 139, 140, 146 and 153.

In another preferred aspect, the CTL epitopes are a mixture of naïve T epitopes (CTL epitopes) and of neo-epitopes (CTL neo-epitopes), advantageously 1 to 15 CTL epitopes and 1 to 15 CTL neo-epitopes. The CTL epitopes are selected from the CTL epitopes of SEQ ID NOs: 3, 8, 22, 23, 30, 31, 32, 36, 42, 48 and 52 and the CTL neo-epitopes are selected from the CTL neo-epitopes of SEQ ID NOs: 56, 59, 60, 66, 70, 76, 77, 78, 79, 83, 91, 92, 125, 135, 139, 140 and 146.

In another preferred aspect, the CTL epitopes are a mixture of naïve T epitopes (CTL epitopes) and of neo-epitopes (CTL neo-epitopes), advantageously 1 to 7 CTL epitopes and 1 to 5 CTL neo-epitopes. The CTL epitopes are selected from the CTL epitopes of SEQ ID NOs: 8, 22, 23, 31, 32, 42 and 52 and the CTL neo-epitopes are selected from the CTL neo-epitopes of SEQ ID NOs: 66, 70, 77, 97 and 146.

The CTL epitopes or neo-epitopes of the vaccine composition target one or several proteins of SARS-CoV, especially selected in the group consisting of Spike glycoprotein (S), Nucleocapsid protein (N), Membrane glycoprotein (M) and ORfs, more particularly Protein 3a, nsp3, nsp4, nsp6, nsp12, nsp13, nsp14 and nsp16.

Accordingly, the CTL epitopes or neo-epitopes of the vaccine composition are selected for targeting 1, 2, 3, 4, 5, 6, 7 or 8 of Spike glycoprotein (S), Nucleocapsid protein (N), Membrane glycoprotein (M), Protein 3a, nsp3, nsp4, nsp6, nsp12, nsp13, nsp14 and nsp16, preferably at least 5, 6, 7, 8, 9, 10, or 11 proteins of SARS-CoV.

Optionally, for each protein, the CTL (neo)epitopes are selected in the following groups:
the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146;
the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23;
the CTL (neo)epitope targeting M is SEQ ID NO: 66;
the CTL (neo)epitopes targeting Protein 3a are selected from one of the groups consisting of (i) SEQ ID NOs: 3, 97 and 101; and (ii) SEQ ID NO: 97;
the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;
the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;
the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;
the CTL (neo)epitopes targeting nsp12 are selected from one of the groups consisting of (i) SEQ ID NOs: 32, 33 and 153; and (ii) SEQ ID NO: 32;
the CTL (neo)epitopes targeting nsp13 are selected from one of the groups consisting of (i) SEQ ID NOs: 22 and 120; and (ii) SEQ ID NO: 22;
the CTL epitope targeting nsp14 is SEQ ID NO: 31; and
the CTL epitope targeting nsp16 is SEQ ID NO: 52.

Optionally, the CTL epitopes or neo-epitopes of the vaccine composition are selected for targeting at least:

Spike glycoprotein (S) and Nucleocapsid protein (N); Spike glycoprotein (S) and Membrane glycoprotein (M); Spike glycoprotein (S) and Protein 3a; Spike glycoprotein (S) and nsp4; Spike glycoprotein (S) and nsp3; Spike glycoprotein (S) and nsp6; Spike glycoprotein (S) and nsp12; Spike glycoprotein (S) and nsp13; Spike glycoprotein (S) and nsp14; Spike glycoprotein (S) and nsp16; Nucleocapsid protein (N) and Membrane glycoprotein (M); Nucleocapsid protein (N) and Protein 3a; Nucleocapsid protein (N) and nsp4; Nucleocapsid protein (N) and nsp3; Nucleocapsid protein (N) and nsp6; Nucleocapsid protein (N) and nsp12; Nucleocapsid protein (N) and nsp13; Nucleocapsid protein (N) and nsp14; Nucleocapsid protein (N) and nsp16; Spike glycoprotein (S), Nucleocapsid protein (N) and Membrane glycoprotein (M); Spike glycoprotein (S), Nucleocapsid protein (N) and Protein 3a; Spike glycoprotein (S), Nucleocapsid protein (N) and nsp4; Spike glycoprotein (S), Nucleocapsid protein (N) and nsp3; Spike glycoprotein (S), Spike glycoprotein (S), Nucleocapsid protein (N) and nsp6; Spike glycoprotein (S), Nucleocapsid protein (N) and nsp12; Spike glycoprotein (S), Nucleocapsid protein (N) and nsp13; Spike glycoprotein (S), Nucleocapsid protein (N) and nsp14; Spike glycoprotein (S), Nucleocapsid protein (N) and nsp16; Membrane glycoprotein (M) and Protein 3a; Membrane glycoprotein (M) and nsp4; Membrane glycoprotein (M) and nsp3; Membrane glycoprotein (M) and nsp6; Membrane glycoprotein (M) and nsp12; Membrane glycoprotein (M) and nsp13; Membrane glycoprotein (M) and nsp14; Membrane glycoprotein (M) and nsp16; Spike glycoprotein (S), Membrane glycoprotein (M) and Protein 3a; Spike glycoprotein (S), Membrane glycoprotein (M) and nsp4; Spike glycoprotein (S), Membrane glycoprotein (M) and nsp3; Spike glycoprotein (S), Membrane glycoprotein (M) and nsp6; Spike glycoprotein (S), Membrane glycoprotein (M) and nsp12; Spike glycoprotein (S), Membrane glycoprotein (M) and nsp13; Spike glycoprotein (S), Membrane glycoprotein (M) and nsp14; Spike glycoprotein (S), Membrane glycoprotein (M) and nsp16; Nucleocapsid protein (N), Membrane glycoprotein (M) and Protein 3a; Nucleocapsid protein (N), Membrane glycoprotein (M) and nsp4; Nucleocapsid protein (N), Membrane glycoprotein (M) and nsp3; Nucleocapsid protein (N), Membrane glycoprotein (M) and nsp6; Nucleocapsid protein (N), Membrane glycoprotein (M) and nsp12; Nucleocapsid protein (N), Membrane glycoprotein (M) and nsp13; Nucleocapsid protein (N), Membrane glycoprotein (M) and nsp14; Nucleocapsid protein (N), Membrane glycoprotein (M) and nsp16; Spike glycoprotein (S), Nucleocapsid protein (N), Membrane glycoprotein (M) and Protein 3a; Spike glycoprotein (S), Nucleocapsid protein (N), Membrane glycoprotein (M) and nsp4; Spike glycoprotein (S), Nucleocapsid protein (N), Membrane glycoprotein (M) and nsp3; Spike glycoprotein (S), Nucleocapsid protein (N), Membrane glycoprotein (M) and nsp6; Spike glycoprotein (S), Nucleocapsid protein (N), Membrane glycoprotein (M) and nsp12; Spike glycoprotein (S), Nucleocapsid protein (N), Membrane glycoprotein (M) and nsp13; Spike glycoprotein (S), Nucleocapsid protein (N), Membrane glycoprotein (M) and nsp14; or Spike glycoprotein (S), Nucleocapsid protein (N), Membrane glycoprotein (M) and nsp16.

In a particular aspect, the CTL epitopes or neo-epitopes of the vaccine composition are selected for targeting one or several of the following groups
Spike glycoprotein (S) and Nucleocapsid protein (N) and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23;

Spike glycoprotein (S) and Membrane glycoprotein (M) and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitope targeting M is SEQ ID NO: 66;

Spike glycoprotein (S) and Protein 3a and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting Protein 3a are selected from one of the groups consisting of (i) SEQ ID NOs: 3, 97 and 101; and (ii) SEQ ID NO: 97;

Spike glycoprotein (5) and nsp3 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;

Spike glycoprotein (5) and nsp4 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;

Spike glycoprotein (5) and nsp6 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;

Spike glycoprotein (5) and nsp12 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting nsp12 are selected from one of the groups consisting of (i) SEQ ID NOs: 32, 33 and 153; and (ii) SEQ ID NO: 32;

Spike glycoprotein (5) and nsp13, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting nsp13 are selected from one of the groups consisting of (i) SEQ ID NOs: 22 and 120; and (ii) SEQ ID NO: 22;

Spike glycoprotein (5) and nsp14 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitope targeting nsp14 is SEQ ID NO: 31;

Spike glycoprotein (5) and nsp16 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitope targeting nsp16 is SEQ ID NO: 52;

Nucleocapsid protein (N) and Membrane glycoprotein (M) and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitope targeting M is SEQ ID NO: 66;

Nucleocapsid protein (N) and Protein 3a and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting Protein 3a are selected from one of the groups consisting of (i) SEQ ID NOs: 3, 97 and 101; and (ii) SEQ ID NO: 97;

Nucleocapsid protein (N) and nsp3 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;

Nucleocapsid protein (N) and nsp4 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;

Nucleocapsid protein (N) and nsp6 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;

Nucleocapsid protein (N) and nsp12 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp12 are selected from one of the groups consisting of (i) SEQ ID NOs: 32, 33 and 153; and (ii) SEQ ID NO: 32;

Nucleocapsid protein (N) and nsp13 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp13 are selected from one of the groups consisting of (i) SEQ ID NOs: 22 and 120; and (ii) SEQ ID NO: 22;

Nucleocapsid protein (N) and nsp14 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL epitope targeting nsp14 is SEQ ID NO: 31;

Nucleocapsid protein (N) and nsp16 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL epitope targeting nsp16 is SEQ ID NO: 52;

Spike glycoprotein (5), Nucleocapsid protein (N) and Membrane glycoprotein (M), and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitope targeting M is SEQ ID NO: 66;

Spike glycoprotein (5), Nucleocapsid protein (N) and Protein 3a and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting Protein 3a are selected from one of the groups consisting of (i) SEQ ID NOs: 3, 97 and 101; and (ii) SEQ ID NO: 97;

Spike glycoprotein (5), Nucleocapsid protein (N) and nsp3 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;

Spike glycoprotein (5), Nucleocapsid protein (N) and nsp4 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;

Spike glycoprotein (5), Nucleocapsid protein (N) and nsp6 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;

Spike glycoprotein (5), Nucleocapsid protein (N) and nsp12 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp12 are selected from one of the groups consisting of (i) SEQ ID NOs: 32, 33 and 153; and (ii) SEQ ID NO: 32;

Spike glycoprotein (5), Nucleocapsid protein (N) and nsp13 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70, and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp13 are selected from one of the groups consisting of (i) SEQ ID NOs: 22 and 120; and (ii) SEQ ID NO: 22;

Spike glycoprotein (5), Nucleocapsid protein (N) and nsp14 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitope targeting nsp14 is SEQ ID NO: 31;

Spike glycoprotein (5), Nucleocapsid protein (N) and nsp16 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL epitope targeting nsp16 is SEQ ID NO: 52;

Membrane glycoprotein (M) and Protein 3a and wherein the CTL (neo)epitope targeting M is SEQ ID NO: 66; and the CTL (neo)epitopes targeting Protein 3a are selected from one of the groups consisting of (i) SEQ ID NOs: 3, 97 and 101; and (ii) SEQ ID NO: 97;

Membrane glycoprotein (M) and nsp3, and wherein the CTL (neo)epitope targeting M is SEQ ID NO: 66; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;

Membrane glycoprotein (M) and nsp4 and wherein the CTL (neo)epitope targeting M is SEQ ID NO: 66; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;

Membrane glycoprotein (M) and nsp6 and wherein the CTL (neo)epitope targeting M is SEQ ID NO: 66; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;

Membrane glycoprotein (M) and nsp12 and wherein the CTL (neo)epitope targeting M is SEQ ID NO: 66; and the CTL (neo)epitopes targeting nsp12 are selected from one of the groups consisting of (i) SEQ ID NOs: 32, 33 and 153; and (ii) SEQ ID NO: 32;

Membrane glycoprotein (M) and nsp13 and wherein the CTL (neo)epitope targeting M is SEQ ID NO: 66; and the CTL (neo)epitopes targeting nsp13 are selected from one of the groups consisting of (i) SEQ ID NOs: 22 and 120; and (ii) SEQ ID NO: 22;

Membrane glycoprotein (M) and nsp14 and wherein the CTL (neo)epitope targeting M is SEQ ID NO: 66; and the CTL (neo)epitope targeting nsp14 is SEQ ID NO: 31;

Membrane glycoprotein (M) and nsp16 and wherein the CTL (neo)epitope targeting M is SEQ ID NO: 66; and the CTL epitope targeting nsp16 is SEQ ID NO: 52;

Spike glycoprotein (5), Membrane glycoprotein (M) and Protein 3a, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL (neo)epitopes targeting Protein 3a are selected from one of the groups consisting of (i) SEQ ID NOs: 3, 97 and 101; and (ii) SEQ ID NO: 97;

Spike glycoprotein (5), Membrane glycoprotein (M) and nsp3, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;

Spike glycoprotein (5), Membrane glycoprotein (M) and nsp4, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;

Spike glycoprotein (5), Membrane glycoprotein (M) and nsp6, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;

Spike glycoprotein (5), Membrane glycoprotein (M) and nsp12, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL (neo)epitopes targeting nsp12 are selected from one of the groups consisting of (i) SEQ ID NOs: 32, 33 and 153; and (ii) SEQ ID NO: 32;

Spike glycoprotein (5), Membrane glycoprotein (M) and nsp13, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL (neo)epitopes targeting nsp13 are selected from one of the groups consisting of (i) SEQ ID NOs: 22 and 120; and (ii) SEQ ID NO: 22;

Spike glycoprotein (5), Membrane glycoprotein (M) and nsp14, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL epitope targeting nsp14 is SEQ ID NO: 31;

Spike glycoprotein (5), Membrane glycoprotein (M) and nsp16, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL epitope targeting nsp16 is SEQ ID NO: 52;

Nucleocapsid protein (N), Membrane glycoprotein (M) and Protein 3a, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting Protein 3a are selected from one of the groups consisting of (i) SEQ ID NOs: 3, 97 and 101; and (ii) SEQ ID NO: 97;

Nucleocapsid protein (N), Membrane glycoprotein (M) and nsp3, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;

Nucleocapsid protein (N), Membrane glycoprotein (M) and nsp4, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;

Nucleocapsid protein (N), Membrane glycoprotein (M) and nsp6, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;

Nucleocapsid protein (N), Membrane glycoprotein (M) and nsp12, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp12 are selected from one of the groups consisting of (i) SEQ ID NOs: 32, 33 and 153; and (ii) SEQ ID NO: 32;

Nucleocapsid protein (N), Membrane glycoprotein (M) and nsp13, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp13 are selected from one of the groups consisting of (i) SEQ ID NOs: 22 and 120; and (ii) SEQ ID NO: 22;

Nucleocapsid protein (N), Membrane glycoprotein (M) and nsp14, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL epitope targeting nsp14 is SEQ ID NO: 31;

Nucleocapsid protein (N), Membrane glycoprotein (M) and nsp16, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL epitope targeting nsp16 is SEQ ID NO: 52;

Spike glycoprotein (5), Nucleocapsid protein (N), Membrane glycoprotein (M) and Protein 3a, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting Protein 3a are selected from one of the groups consisting of (i) SEQ ID NOs: 3, 97 and 101; and (ii) SEQ ID NO: 97;

Spike glycoprotein (5), Nucleocapsid protein (N), Membrane glycoprotein (M) and nsp3, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;

Spike glycoprotein (5), Nucleocapsid protein (N), Membrane glycoprotein (M) and nsp4, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;

Spike glycoprotein (5), Nucleocapsid protein (N), Membrane glycoprotein (M) and nsp6, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;

Spike glycoprotein (5), Nucleocapsid protein (N), Membrane glycoprotein (M) and nsp12, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp12 are selected from one of the groups consisting of (i) SEQ ID NOs: 32, 33 and 153; and (ii) SEQ ID NO: 32;

Spike glycoprotein (5), Nucleocapsid protein (N), Membrane glycoprotein (M) and nsp13, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp13 are selected from one of the groups consisting of (i) SEQ ID NOs: 22 and 120; and (ii) SEQ ID NO: 22;

Spike glycoprotein (5), Nucleocapsid protein (N), Membrane glycoprotein (M) and nsp14, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL epitope targeting nsp14 is SEQ ID NO: 31; and

Spike glycoprotein (5), Nucleocapsid protein (N), Membrane glycoprotein (M) and nsp16, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL epitope targeting nsp16 is SEQ ID NO: 52.

In a particular aspect, the vaccine composition comprises CTL (neo)epitopes targeting at least 5, 6, 7, 8, 9, 10, or 11 proteins of SARS-CoV selected in the group consisting of Spike glycoprotein (5), Nucleocapsid protein (N), Membrane glycoprotein (M), Protein 3a, nsp3, nsp4, nsp6, nsp12, nsp13, nsp14 and nsp16; and for each proteins the CTL epitopes are selected in the following groups:
the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146;
the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23;
the CTL (neo)epitope targeting M is SEQ ID NO: 66;
the CTL (neo)epitopes targeting Protein 3a are selected from one of the groups consisting of (i) SEQ ID NOs: 3, 97 and 101; and (ii) SEQ ID NO: 97;
the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;
the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;
the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;
the CTL (neo)epitopes targeting nsp12 are selected from one of the groups consisting of (i) SEQ ID NOs: 32, 33 and 153; and (ii) SEQ ID NO: 32;
the CTL (neo)epitopes targeting nsp13 are selected from one of the groups consisting of (i) SEQ ID NOs: 22 and 120; and (ii) SEQ ID NO: 22;
the CTL (neo)epitope targeting nsp14 is SEQ ID NO: 31; and
the CTL (neo)epitope targeting nsp16 is SEQ ID NO: 52.

Optionally, for each targeted protein, the vaccine composition comprises at least one CTL epitope and at least one CTL neoepitope. Optionally, for each targeted protein, the vaccine composition comprises at least one CTL epitope and at least two CTL neoepitopes. Optionally, for each targeted protein, the vaccine composition comprises at least two CTL neoepitopes. Optionally, for each targeted protein, the vaccine composition independently for the different targeted proteins comprises:
at least one CTL epitope selected from Table 1 and at least one CTL neoepitope selected from Table 2 and/or 3; or
at least one CTL epitope and at least two CTL neoepitopes selected from Table 2 and/or 3; or
at least two CTL neoepitopes selected from Table 2 and/or 3.

Optionally, for each targeted protein, the vaccine composition independently for the different targeted proteins comprises:
at least one CTL epitope selected in the group consisting of SEQ ID NOs: 3, 8, 20, 22, 23, 30, 31, 32, 33, 34, 36, 42, 48, 49, and 52 and at least one CTL neoepitope selected in the group consisting of SEQ ID NOs: 56, 59, 60, 66, 67, 70, 74, 75, 76, 77, 78, 79, 83, 84, 85, 86, 90, 91, 92, 95, 97, 101, 104, 105, 113, 120, 125, 135, 139, 140, 146, and 153; or
at least one CTL epitope selected in the group consisting of SEQ ID NOs: 3, 8, 20, 22, 23, 30, 31, 32, 33, 34, 36, 42, 48, 49, and 52 and at least two CTL neoepitopes selected in the group consisting of SEQ ID NOs: 56, 59, 60, 66, 67, 70, 74, 75, 76, 77, 78, 79, 83, 84, 85, 86, 90, 91, 92, 95, 97, 101, 104, 105, 113, 120, 125, 135, 139, 140, 146, and 153; or
at least two CTL neoepitopes selected in the group consisting of SEQ ID NOs: 56, 59, 60, 66, 67, 70, 74, 75, 76, 77, 78, 79, 83, 84, 85, 86, 90, 91, 92, 95, 97, 101, 104, 105, 113, 120, 125, 135, 139, 140, 146, and 153.

Optionally, for each targeted protein, the vaccine composition independently for the different targeted proteins comprises:
at least one CTL epitope selected in the group consisting of SEQ ID NOs: 3, 8, 22, 23, 30, 31, 32, 36, 42, 48, and 52 and at least one CTL neoepitope selected in the group consisting of SEQ ID NOs: 56, 59, 60, 66, 70, 76, 77, 78, 79, 83, 91, 92, 125, 135, 139, 140, and 146; or
at least one CTL epitope selected in the group consisting of SEQ ID NOs: 3, 8, 22, 23, 30, 31, 32, 36, 42, 48, and 52 and at least two CTL neoepitopes selected in the group consisting of SEQ ID NOs: 56, 59, 60, 66, 70, 76, 77, 78, 79, 83, 91, 92, 125, 135, 139, 140, and 146; or
at least two CTL neoepitopes selected in the group consisting of SEQ ID NOs: 56, 59, 60, 66, 70, 76, 77, 78, 79, 83, 91, 92, 125, 135, 139, 140, and 146.

Optionally, for each targeted protein, the vaccine composition independently for the different targeted proteins comprises:
at least one CTL epitope selected in the group consisting of SEQ ID NOs: 8, 22, 23, 31, 32, 42 and 52 and at least one CTL neoepitope selected in the group consisting of SEQ ID NOs: 66, 70, 77, 97 and 146; or
at least one CTL epitope selected in the group consisting of SEQ ID NOs: 8, 22, 23, 31, 32, 42 and 52 and at least two CTL neoepitopes selected in the group consisting of SEQ ID NOs: 66, 70, 77, 97 and 146; or
at least two CTL neoepitopes selected in the group consisting of SEQ ID NOs: 66, 70, 77, 97 and 146.

Optionally, the vaccine composition comprises CTL epitopes/neoepitopes targeting Spike glycoprotein (S) and the vaccine composition comprises:
at least one CTL epitope targeting Spike glycoprotein (S) selected from Table 1 and at least one CTL neoepitope targeting Spike glycoprotein (S) selected from Table 2 and/or 3; or
at least one CTL epitope targeting Spike glycoprotein (S) selected from Table 1 and at least two CTL neoepitopes targeting Spike glycoprotein (S) selected from Table 2 and/or 3; or
at least two CTL neoepitopes targeting Spike glycoprotein (S) selected from Table 2 and/or 3.

Optionally, the vaccine composition comprises CTL epitopes/neoepitopes targeting Spike glycoprotein (S) and the vaccine composition comprises:
at least one CTL epitope targeting Spike glycoprotein (S) selected from the group consisting of SEQ ID NOs: 34 and 48 and at least one CTL neoepitope targeting Spike glycoprotein (S) selected from the group consisting of SEQ ID NOs: 56, 60, 70, 74, 84, 86, 91, 92, 104, and 146; or
at least one CTL epitope targeting Spike glycoprotein (S) selected from the group consisting of SEQ ID NOs: 34 and 48 and at least two CTL neoepitopes targeting Spike glycoprotein (S) selected from the group consisting of SEQ ID NOs: 56, 60, 70, 74, 84, 86, 91, 92, 104, and 146; or
at least two CTL neoepitopes targeting Spike glycoprotein (S) selected from the group consisting of SEQ ID NOs: 56, 60, 70, 74, 84, 86, 91, 92, 104, and 146.

Optionally, the vaccine composition comprises CTL epitopes/neoepitopes targeting Spike glycoprotein (S) and the vaccine composition comprises:
one CTL epitope targeting Spike glycoprotein (S) of SEQ ID NO 48 and at least one CTL neoepitope targeting Spike glycoprotein (S) selected from the group consisting of SEQ ID NOs: 56, 60, 70, 91, 92, and 146; or
one CTL epitope targeting Spike glycoprotein (S) of SEQ ID NO 48 and at least two CTL neoepitopes targeting Spike glycoprotein (S) selected from the group consisting of SEQ ID NOs: 56, 60, 70, 91, 92, and 146; or
at least two CTL neoepitopes targeting Spike glycoprotein (S) selected from the group consisting of SEQ ID NOs: 56, 60, 70, 91, 92, and 146.

Optionally, the vaccine composition comprises CTL epitopes/neoepitopes targeting Spike glycoprotein (S) and the vaccine composition comprises:
one CTL epitope targeting Spike glycoprotein (S) of SEQ ID NO 48 and at least one CTL neoepitope targeting Spike glycoprotein (S) selected from the group consisting of SEQ ID NOs: 70 and 146; or
at least one CTL epitope targeting Spike glycoprotein (S) of SEQ ID NO 48 and two CTL neoepitopes targeting Spike glycoprotein (S) of SEQ ID NOs: 70 and 146; or
two CTL neoepitopes targeting Spike glycoprotein (S) selected from the group consisting of SEQ ID NOs: 70 and 146.

Optionally, the vaccine composition comprises CTL epitopes/neoepitopes targeting Nucleocapsid protein (N) and the vaccine composition comprises:
at least one CTL epitope targeting Nucleocapsid protein (N) selected from Table 1 and at least one CTL neoepitope targeting Nucleocapsid protein (N) selected from Table 2 and/or 3; or
at least one CTL epitope targeting Nucleocapsid protein (N) selected from Table 1 and at least two CTL neoepitopes targeting Nucleocapsid protein (N) selected from Table 2 and/or 3; or
at least two CTL neoepitopes targeting Nucleocapsid protein (N) selected from Table 2 and/or 3.

Optionally, the vaccine composition comprises CTL epitopes/neoepitopes targeting Nucleocapsid protein (N) and the vaccine composition comprises:
one CTL epitope targeting Nucleocapsid protein (N) of SEQ ID NO: 23 and at least one CTL neoepitope targeting Nucleocapsid protein (N) selected from the group consisting of SEQ ID NOs: 67, 75, 79, 85, and 113; or
one CTL epitope targeting Nucleocapsid protein (N) of SEQ ID NO: 23 and at least two CTL neoepitopes targeting Nucleocapsid protein (N) selected from the group consisting of SEQ ID NOs: 67, 75, 79, 85, and 113; or
at least two CTL neoepitopes targeting Nucleocapsid protein (N) selected from the group consisting of SEQ ID NOs: 67, 75, 79, 85, and 113.

Optionally, the vaccine composition comprises CTL epitopes/neoepitopes targeting Nucleocapsid protein (N) and the vaccine composition comprises one CTL epitope targeting Nucleocapsid protein (N) of SEQ ID NO: 23 and one CTL neoepitope targeting Nucleocapsid protein (N) of SEQ ID NO:79.

Optionally, the vaccine composition comprises CTL epitopes/neoepitopes targeting Membrane glycoprotein (M) and the vaccine composition comprises:
at least one CTL epitope targeting Membrane glycoprotein (M) selected from Table 1 and at least one CTL neoepitope targeting Membrane glycoprotein (M) selected from Table 2 and/or 3; or
at least one CTL epitope targeting Membrane glycoprotein (M) selected from Table 1 and at least two CTL neoepitopes targeting Membrane glycoprotein (M) selected from Table 2 and/or 3; or
at least two CTL neoepitopes targeting Membrane glycoprotein (M) selected from Table 2 and/or 3.

Optionally, the vaccine composition comprises CTL epitopes/neoepitopes targeting Membrane glycoprotein (M) and the vaccine composition comprises one CTL neoepitope targeting Membrane glycoprotein (M) of SEQ ID NO: 66.

Optionally, the vaccine composition comprises CTL epitopes/neoepitopes targeting at least one ORF selected from the group consisting of Protein 3a, nsp4, nsp3, nsp6, nsp12, nsp13, nsp14 and nsp16, and the vaccine composition comprises:
at least one CTL epitope targeting at least one ORF selected from the group consisting of Protein 3a, nsp4, nsp3, nsp6, nsp12, nsp13, nsp14 and nsp16 and selected from Table 1 and at least one CTL neoepitope targeting at least one ORF selected from the group consisting of Protein 3a, nsp4, nsp3, nsp6, nsp12, nsp13, nsp14 and nsp16 and selected from Table 2 and/or 3; or
at least one CTL epitope and at least two CTL neoepitopes targeting at least one ORF selected from the group consisting of Protein 3a, nsp4, nsp3, nsp6, nsp12, nsp13, nsp14 and nsp16 and selected from Table 2 and/or 3; or
at least two CTL neoepitopes targeting at least one ORF selected from the group consisting of Protein 3a, nsp4, nsp3, nsp6, nsp12, nsp13, nsp14 and nsp16 and selected from Table 2 and/or 3.

Optionally, the vaccine composition comprises CTL epitopes/neoepitopes targeting at least one ORF selected from the group consisting of Protein 3a, nsp4, nsp3, nsp6, nsp12, nsp13, nsp14 and nsp16, and the vaccine composition comprises:
at least one CTL epitope targeting at least one ORF selected from the group consisting of Protein 3a, nsp4, nsp3, nsp6, nsp12, nsp13, nsp14 and nsp16 and selected from the group consisting of SEQ ID NOs: 3, 8, 20, 22, 30, 31, 32, 33, 36, 42, 49, and 52 and at least one CTL neoepitope targeting at least one ORF selected from the group consisting of Protein 3a, nsp4, nsp3, nsp6, nsp12, nsp13, nsp14 and nsp16 and selected from the group consisting of SEQ ID NOs: 59, 76, 77, 78, 83, 90, 95, 97, 101, 105, 120, 125, 135, 139, 140, 153; or
at least one CTL epitope targeting at least one ORF selected from the group consisting of Protein 3a, nsp4, nsp3, nsp6, nsp12, nsp13, nsp14 and nsp16 and selected from the group consisting of SEQ ID NOs: 3, 8, 20, 22, 30, 31, 32, 33, 36, 42, 49, and 52 and at least two CTL neoepitopes targeting at least one ORF selected from the group consisting of Protein 3a, nsp4, nsp3, nsp6, nsp12, nsp13, nsp14 and nsp16 and selected from the group consisting of SEQ ID NOs: 59, 76, 77, 78, 83, 90, 95, 97, 101, 105, 120, 125, 135, 139, 140, 153; or
at least two CTL neoepitopes targeting at least one ORF selected from the group consisting of Protein 3a, nsp4, nsp3, nsp6, nsp12, nsp13, nsp14 and nsp16 and selected from the group consisting of SEQ ID NOs: 59, 76, 77, 78, 83, 90, 95, 97, 101, 105, 120, 125, 135, 139, 140, 153.

Optionally, the vaccine composition comprises CTL epitopes/neoepitopes targeting at least one ORF selected from the group consisting of Protein 3a, nsp4, nsp3, nsp6, nsp12, nsp13, nsp14 and nsp16, and the vaccine composition comprises:
at least one CTL epitope targeting at least one ORF selected from the group consisting of Protein 3a, nsp4, nsp3, nsp6, nsp12, nsp13, nsp14 and nsp16 and selected from the group consisting of SEQ ID NOs: 3, 8, 22, 30, 31, 32, 36, 42, and 52 and at least one CTL neoepitope targeting at least one ORF selected from the group consisting of Protein 3a, nsp4, nsp3, nsp6, nsp12, nsp13, nsp14 and nsp16 and selected from the group consisting of SEQ ID NOs: 59, 76, 77, 78, 83, 125, 135, 139, and 140; or
at least one CTL epitope targeting at least one ORF selected from the group consisting of Protein 3a, nsp4, nsp3, nsp6, nsp12, nsp13, nsp14 and nsp16 and selected from the group consisting of SEQ ID NOs: 3, 8, 22, 30, 31, 32, 36, 42, and 52 and at least two CTL neoepitopes targeting at least one ORF selected from the group consisting of Protein 3a, nsp4, nsp3, nsp6, nsp12, nsp13, nsp14 and nsp16 and selected from the group consisting of SEQ ID NOs: 59, 76, 77, 78, 83, 125, 135, 139, and 140; or
at least two CTL neoepitopes targeting at least one ORF selected from the group consisting of Protein 3a, nsp4, nsp3, nsp6, nsp12, nsp13, nsp14 and nsp16 and selected from the group consisting of SEQ ID NOs: 59, 76, 77, 78, 83, 125, 135, 139, and 140.

Optionally, the vaccine composition comprises CTL epitopes/neoepitopes targeting at least one ORF selected from the group consisting of Protein 3a, nsp4, nsp3, nsp6, nsp12, nsp13, nsp14 and nsp16, and the vaccine composition comprises:
at least one CTL epitope targeting at least one ORF selected from the group consisting of Protein 3a, nsp4, nsp3, nsp6, nsp12, nsp13, nsp14 and nsp16 and selected from the group consisting of SEQ ID NOs: 8, 22, 31, 32, 42 and 52 and at least one CTL neoepitope targeting at least one ORF selected from the group consisting of nsp3 and nsp6 and selected from the group consisting of SEQ ID NOs: 77 and 97; or
at least one CTL epitope targeting at least one ORF selected from the group consisting of Protein 3a, nsp4, nsp3, nsp6, nsp12, nsp13, nsp14 and nsp16 and selected from the group consisting of SEQ ID NOs: 8, 22, 31, 32, 42 and 52 and two CTL neoepitopes targeting nsp3 and nsp6 and of SEQ ID NOs: 77 and 97; or
two CTL neoepitopes targeting nsp3 and nsp6 and of SEQ ID NOs: 77 and 97.

Optionally, the vaccine composition comprises CTL epitopes/neoepitopes targeting Spike glycoprotein (S) and Nucleocapsid protein (N) and the vaccine composition comprises:
at least one CTL epitope targeting Spike glycoprotein (S) selected from Table 1 and at least one CTL neoepitope targeting Spike glycoprotein (S) selected from Table 2 and/or 3; and at least one CTL epitope targeting Nucleocapsid protein (N) selected from Table 1 and at least one CTL neoepitope targeting Nucleocapsid protein (N) selected from Table 2 and/or 3; or
at least one CTL epitope targeting Spike glycoprotein (S) selected from Table 1 and at least one CTL neoepitope targeting Spike glycoprotein (S) selected from Table 2 and/or 3; and at least one CTL epitope targeting Nucleocapsid protein (N) selected from Table 1 and at least two CTL neoepitopes targeting Nucleocapsid protein (N) selected from Table 2 and/or 3; or
at least one CTL epitope targeting Spike glycoprotein (S) selected from Table 1 and at least one CTL neoepitope targeting Spike glycoprotein (S) selected from Table 2 and/or 3; and at least two CTL neoepitopes targeting Nucleocapsid protein (N) selected from Table 2 and/or 3;
at least one CTL epitope targeting Spike glycoprotein (S) selected from Table 1 and at least two CTL neoepitopes targeting Spike glycoprotein (S) selected from Table 2 and/or 3; and at least one CTL epitope targeting Nucleocapsid protein (N) selected from Table 1 and at least one CTL neoepitope targeting Nucleocapsid protein (N) selected from Table 2 and/or 3; or
at least one CTL epitope targeting Spike glycoprotein (S) selected from Table 1 and at least two CTL neoepitopes targeting Spike glycoprotein (S) selected from Table 2 and/or 3; and at least one CTL epitope targeting Nucleocapsid protein (N) selected from Table 1 and at least two CTL neoepitopes targeting Nucleocapsid protein (N) selected from Table 2 and/or 3; or
at least one CTL epitope targeting Spike glycoprotein (S) selected from Table 1 and at least two CTL neoepitopes targeting Spike glycoprotein (S) selected from Table 2 and/or 3; and at least two CTL neoepitopes targeting Nucleocapsid protein (N) selected from Table 2 and/or 3;
at least two CTL neoepitopes targeting Spike glycoprotein (S) selected from Table 2 and/or 3; and at least one CTL epitope targeting Nucleocapsid protein (N) selected from Table 1 and at least one CTL neoepitope targeting Nucleocapsid protein (N) selected from Table 2 and/or 3; or
at least two CTL neoepitopes targeting Spike glycoprotein (S) selected from Table 2 and/or 3; and at least one CTL epitope targeting Nucleocapsid protein (N) selected from Table 1 and at least two CTL neoepitopes targeting Nucleocapsid protein (N) selected from Table 2 and/or 3; or
at least two CTL neoepitopes targeting Spike glycoprotein (S) selected from Table 2 and/or 3; and at least two CTL neoepitopes targeting Nucleocapsid protein (N) selected from Table 2 and/or 3.

Optionally, the vaccine composition comprises CTL epitopes/neoepitopes targeting Spike glycoprotein (S) and Nucleocapsid protein (N) and the vaccine composition comprises:
at least one CTL epitope targeting Spike glycoprotein (S) selected from the group consisting of SEQ ID NOs: 34 and 48 and at least one CTL neoepitope targeting Spike glycoprotein (S) selected from the group consisting of SEQ ID NOs: 56, 60, 70, 74, 84, 86, 91, 92, 104, and 146; and one CTL epitope targeting Nucleocapsid protein (N) of SEQ ID NO: 23 and at least one CTL neoepitope targeting Nucleocapsid protein (N) selected from the group consisting of SEQ ID NOs: 67, 75, 79, 85, and 113; or
at least one CTL epitope targeting Spike glycoprotein (S) selected from the group consisting of SEQ ID NOs: 34 and 48 and at least one CTL neoepitope targeting Spike glycoprotein (S) selected from the group consisting of SEQ ID NOs: 56, 60, 70, 74, 84, 86, 91, 92, 104, and 146; and one CTL epitope targeting Nucleocapsid protein (N) of SEQ ID NO: 23 and at least two CTL neoepitopes targeting Nucleocapsid protein (N) selected from the group consisting of SEQ ID NOs: 67, 75, 79, 85, and 113; or
at least one CTL epitope targeting Spike glycoprotein (S) selected from the group consisting of SEQ ID NOs: 34 and 48 and at least one CTL neoepitope targeting Spike glycoprotein (S) selected from the group consisting of SEQ ID NOs: 56, 60, 70, 74, 84, 86, 91, 92, 104, and 146; and at least two CTL neoepitopes targeting Nucleocapsid protein (N) selected from the group consisting of SEQ ID NOs: 67, 75, 79, 85, and 113;
at least one CTL epitope targeting Spike glycoprotein (S) selected from the group consisting of SEQ ID NOs: 34 and 48 and at least two CTL neoepitopes targeting Spike glycoprotein (S) selected from the group consisting of SEQ ID NOs: 56, 60, 70, 74, 84, 86, 91, 92, 104, and 146; and one CTL epitope targeting Nucleocapsid protein (N) of SEQ ID NO: 23 and at least one CTL neoepitope targeting Nucleocapsid protein (N) selected from the group consisting of SEQ ID NOs: 67, 75, 79, 85, and 113; or
at least one CTL epitope targeting Spike glycoprotein (S) selected from the group consisting of SEQ ID NOs: 34 and 48 and at least two CTL neoepitopes targeting Spike glycoprotein (S) selected from the group consisting of SEQ ID NOs: 56, 60, 70, 74, 84, 86, 91, 92, 104, and 146; and one CTL epitope targeting Nucleocapsid protein (N) of SEQ ID NO: 23 and at least two CTL neoepitopes targeting Nucleocapsid protein (N) selected from the group consisting of SEQ ID NOs: 67, 75, 79, 85, and 113; or
at least one CTL epitope targeting Spike glycoprotein (S) selected from the group consisting of SEQ ID NOs: 34 and 48 and at least two CTL neoepitopes targeting Spike glycoprotein (S) selected from the group consisting of SEQ ID NOs: 56, 60, 70, 74, 84, 86, 91, 92, 104, and 146; and at least two CTL neoepitopes targeting Nucleocapsid protein (N) selected from the group consisting of SEQ ID NOs: 67, 75, 79, 85, and 113;
at least two CTL neoepitopes targeting Spike glycoprotein (S) selected from the group consisting of SEQ ID NOs: 56, 60, 70, 74, 84, 86, 91, 92, 104, and 146; and one CTL epitope targeting Nucleocapsid protein (N) of SEQ ID NO: 23 and at least one CTL neoepitope targeting Nucleocapsid protein (N) selected from the group consisting of SEQ ID NOs: 67, 75, 79, 85, and 113; or
at least two CTL neoepitopes targeting Spike glycoprotein (S) selected from the group consisting of SEQ ID NOs: 56, 60, 70, 74, 84, 86, 91, 92, 104, and 1463; and one CTL epitope targeting Nucleocapsid protein (N) of SEQ ID NO: 23 and at least two CTL neoepitopes targeting Nucleocapsid protein (N) selected from the group consisting of SEQ ID NOs: 67, 75, 79, 85, and 113; or
at least two CTL neoepitopes targeting Spike glycoprotein (S) selected from the group consisting of SEQ ID NOs: 56, 60, 70, 74, 84, 86, 91, 92, 104, and 146; and at least two CTL neoepitopes targeting Nucleocapsid protein (N) selected from the group consisting of SEQ ID NOs: 67, 75, 79, 85, and 113.

Optionally, the vaccine composition comprises CTL epitopes/neoepitopes targeting Spike glycoprotein (S) and Membrane glycoprotein (M) and the vaccine composition comprises:
at least one CTL epitope targeting Spike glycoprotein (S) selected from Table 1 and at least one CTL neoepitope targeting Spike glycoprotein (S) selected from Table 2 and/or 3; and at least one CTL epitope targeting Membrane glycoprotein (M) selected from Table 1 and at least one CTL neoepitope targeting Membrane glycoprotein (M) selected from Table 2 and/or 3; or
at least one CTL epitope targeting Spike glycoprotein (S) selected from Table 1 and at least one CTL neoepitope targeting Spike glycoprotein (S) selected from Table 2 and/or 3; and at least one CTL epitope targeting Membrane glycoprotein (M) selected from Table 1 and at least two CTL neoepitopes targeting Membrane glycoprotein (M) selected from Table 2 and/or 3; or
at least one CTL epitope targeting Spike glycoprotein (S) selected from Table 1 and at least one CTL neoepitope targeting Spike glycoprotein (S) selected from Table 2 and/or 3; and at least two CTL neoepitopes targeting Membrane glycoprotein (M) selected from Table 2 and/or 3;
at least one CTL epitope targeting Spike glycoprotein (S) selected from Table 1 and at least two CTL neoepitopes targeting Spike glycoprotein (S) selected from Table 2 and/or 3; and at least one CTL epitope targeting Membrane glycoprotein (M) selected from Table 1 and at least one CTL neoepitope targeting Membrane glycoprotein (M) selected from Table 2 and/or 3; or
at least one CTL epitope targeting Spike glycoprotein (S) selected from Table 1 and at least two CTL neoepitopes targeting Spike glycoprotein (S) selected from Table 2 and/or 3; and at least one CTL epitope targeting Membrane glycoprotein (M) selected from Table 1 and at least two CTL neoepitopes targeting Membrane glycoprotein (M) selected from Table 2 and/or 3; or
at least one CTL epitope targeting Spike glycoprotein (S) selected from Table 1 and at least two CTL neoepitopes targeting Spike glycoprotein (S) selected from Table 2 and/or 3; and at least two CTL neoepitopes targeting Membrane glycoprotein (M) selected from Table 2 and/or 3;
at least two CTL neoepitopes targeting Spike glycoprotein (S) selected from Table 2 and/or 3; and
at least one CTL epitope targeting Membrane glycoprotein (M) selected from Table 1 and at least one CTL neoepitope targeting Membrane glycoprotein (M) selected from Table 2 and/or 3; or
at least two CTL neoepitopes targeting Spike glycoprotein (S) selected from Table 2 and/or 3; and at least one CTL epitope targeting Membrane glycoprotein (M) selected from Table 1 and at least two CTL neoepitopes targeting Membrane glycoprotein (M) selected from Table 2 and/or 3; or
at least two CTL neoepitopes targeting Spike glycoprotein (S) selected from Table 2 and/or 3; and at least two CTL neoepitopes targeting Membrane glycoprotein (M) selected from Table 2 and/or 3.

Optionally, the vaccine composition comprises CTL epitopes/neoepitopes targeting Spike glycoprotein (S) and Membrane glycoprotein (M) and the vaccine composition comprises:
at least one CTL epitope targeting Spike glycoprotein (S) selected from the group consisting of SEQ ID NOs: 34 and 48 and at least one CTL neoepitope targeting Spike glycoprotein (S) selected from the group consisting of SEQ ID NOs: 56, 60, 70, 74, 84, 86, 91, 92, 104, and 146; and one CTL neoepitope targeting Membrane glycoprotein (M) of SEQ ID NO: 66; or
at least one CTL epitope targeting Spike glycoprotein (S) selected from the group consisting of SEQ ID NOs: 34 and 48 and at least two CTL neoepitopes targeting Spike glycoprotein (S) selected from the group consisting of SEQ ID NOs: 56, 60, 70, 74, 84, 86, 91, 92, 104, and 146; and one CTL neoepitope targeting Membrane glycoprotein (M) of SEQ ID NO: 66; or
at least one CTL epitope targeting Spike glycoprotein (S) selected from the group consisting of SEQ ID NOs: 34 and 48 and at least two CTL neoepitopes targeting Spike glycoprotein (S) selected from the group consisting of SEQ ID NOs: 56, 60, 70, 74, 84, 86, 91, 92, 104, and 146; and one CTL neoepitope targeting Membrane glycoprotein (M) of SEQ ID NO: 66; or
at least two CTL neoepitopes targeting Spike glycoprotein (S) selected from the group consisting of SEQ ID NOs: 56, 60, 70, 74, 84, 86, 91, 92, 104, and 146; and one CTL epitope targeting Nucleocapsid protein (N) of SEQ ID NO: 23 and one CTL neoepitope targeting Membrane glycoprotein (M) of SEQ ID NO: 66.

Optionally, the vaccine composition comprises CTL epitopes/neoepitopes targeting Nucleocapsid protein (N) and Membrane glycoprotein (M) and the vaccine composition comprises:
at least one CTL epitope targeting Nucleocapsid protein (N) selected from Table 1 and at least one CTL neoepitope targeting Nucleocapsid protein (N) selected from Table 2 and/or 3; and at least one CTL epitope targeting Membrane glycoprotein (M) selected from Table 1 and at least one CTL neoepitope targeting Membrane glycoprotein (M) selected from Table 2 and/or 3; or
at least one CTL epitope targeting Nucleocapsid protein (N) selected from Table 1 and at least one CTL neoepitope targeting Nucleocapsid protein (N) selected from Table 2 and/or 3; and at least one CTL epitope targeting Membrane glycoprotein (M) selected from Table 1 and at least two CTL neoepitopes targeting Membrane glycoprotein (M) selected from Table 2 and/or 3; or
at least one CTL epitope targeting Nucleocapsid protein (N) selected from Table 1 and at least one CTL neoepitope targeting Nucleocapsid protein (N) selected from Table 2 and/or 3; and at least two CTL neoepitopes targeting Membrane glycoprotein (M) selected from Table 2 and/or 3;
at least one CTL epitope targeting Nucleocapsid protein (N) selected from Table 1 and at least two CTL neoepitopes targeting Nucleocapsid protein (N) selected from Table 2 and/or 3; and at least one CTL epitope targeting Membrane glycoprotein (M) selected from Table 1 and at least one CTL neoepitope targeting Membrane glycoprotein (M) selected from Table 2 and/or 3; or
at least one CTL epitope targeting Nucleocapsid protein (N) selected from Table 1 and at least two CTL neoepitopes targeting Nucleocapsid protein (N) selected from Table 2 and/or 3; and at least one CTL epitope targeting Membrane glycoprotein (M) selected from Table 1 and at least two CTL neoepitopes targeting Membrane glycoprotein (M) selected from Table 2 and/or 3; or
at least one CTL epitope targeting Nucleocapsid protein (N) selected from Table 1 and at least two CTL neoepitopes targeting Nucleocapsid protein (N) selected from Table 2 and/or 3; and at least two CTL neoepitopes targeting Membrane glycoprotein (M) selected from Table 2 and/or 3;
at least two CTL neoepitopes targeting Nucleocapsid protein (N) selected from Table 2 and/or 3; and at least one CTL epitope targeting Membrane glycoprotein (M) selected from Table 1 and at least one CTL neoepitope targeting Membrane glycoprotein (M) selected from Table 2 and/or 3; or
at least two CTL neoepitopes targeting Nucleocapsid protein (N) selected from Table 2 and/or 3; and at least one CTL epitope targeting Membrane glycoprotein (M) selected from Table 1 and at least two CTL neoepitopes targeting Membrane glycoprotein (M) selected from Table 2 and/or 3; or
at least two CTL neoepitopes targeting Nucleocapsid protein (N) selected from Table 2 and/or 3; and at least two CTL neoepitopes targeting Membrane glycoprotein (M) selected from Table 2 and/or 3.

Optionally, the CTL neoepitope targeting Membrane glycoprotein (M) of SEQ ID NO: 66. Optionally, the CTL epitope targeting Nucleocapsid protein (N) consists of SEQ ID NO: 23 and the CTL neoepitope targeting Nucleocapsid protein (N) are selected from the group consisting of SEQ ID NOs: 67, 75, 79, 85, and 113.

In a particular aspect, the vaccine composition comprises at least 1, 2, 3, 4, 5 or 6 CTL (neo)epitopes selected in one of the groups consisting of (i) SEQ ID NOs: 20, 23, 32, 36, 42, 56, 59, 60, 76, 79, 85, 91, 95, 97, 125, 140 and 146; (ii) SEQ ID NOs: 23, 32, 36, 42, 56, 59, 60, 76, 79, 91, 97, 125, 140 and 146; and (iii) SEQ ID NOs: 23, 32, 42, 97 and 146.

In a very particular aspect, the vaccine composition comprises at least 5, 6, 7, 8, 9, 10, 11, or 12 CTL (neo)epitopes selected from SEQ ID NOs: 8, 22, 23, 31, 32, 42, 52, 66, 70, 77, 97 and 146.

In another very particular aspect, the vaccine composition comprises CTL (neo)epitopes of SEQ ID NOs: 70 and/or 146; 23, and 66, and at least 2, 3, 4, 5, 6, 7 or 8 CTL (neo)epitopes selected from SEQ ID NOs: 8, 22, 31, 32, 42, 52, 77 and 97.

In a very specific aspect, the vaccine composition comprises CTL (neo)epitopes of SEQ ID NOs: 8, 22, 23, 31, 32, 42, 52, 66, 70, 77, 97 and 146. More particularly, the vaccine composition may comprise CTL (neo)epitopes of SEQ ID NOs: 8, 22, 23, 31, 32, 42, 52, 66, 70, 77, 97 and 146 and a T helper peptide, especially PADRE (aKXVAAWTLKAAa with X and a respectively indicating cyclohexylalanine and d-alanine).

For instance, the vaccine composition comprises or consists of one of the following CTL (neo)epitopes:
CTL (neo)epitopes of SEQ ID NOs: 23, 66 and 70 and (a) CTL (neo)epitopes selected from SEQ ID NOs: 8, 22, 31, 32, 42, 52, 77, and 97; (b) CTL (neo)epitopes selected from SEQ ID NOs: 8, 22, 31, 32, 42, 52, and 77; (c) CTL (neo)epitopes selected from SEQ ID NOs: 22, 31, 32, 42, 52, 77 and 97; (d) CTL (neo)epitopes selected from SEQ ID NOs: 8, 31, 32, 42, 52, 77 and 97; (e) CTL (neo)epitopes selected from SEQ ID NOs: 8, 22, 32, 42, 52, 77, and 97; (f) CTL (neo)epitopes selected from SEQ ID NOs: 8, 22, 31, 42, 52, 77, and 97; (g) CTL (neo)epitopes selected from SEQ ID NOs: 8, 22, 31, 32, 42, 77, and 97; or (h) CTL (neo)epitopes selected from SEQ ID NOs: 8, 22, 31, 32, 42, 52, and 97; (i) CTL (neo)epitopes selected from SEQ ID NOs: 8, 22, 31, 32, 52, 77 and 97;
CTL (neo)epitopes of SEQ ID NOs: 23, 66 and 146 and (a) CTL (neo)epitopes selected from SEQ ID NOs: 8, 22, 31, 32, 42, 52, 77, and 97; (b) CTL (neo)epitopes selected from SEQ ID NOs: 8, 22, 31, 32, 42, 52, and 77 140; (c) CTL (neo)epitopes selected from SEQ ID NOs: 22, 31, 32, 42, 52, 77, and 97; (d) CTL (neo)epitopes selected from SEQ ID NOs: 8, 31, 32, 42, 52, 77, and 97; (e) CTL (neo)epitopes selected from SEQ ID NOs: 8, 22, 32, 42, 52, 77, and 97; (f) CTL (neo)epitopes selected from SEQ ID NOs: 8, 22, 31, 42, 52, 77, and 97; (g) CTL (neo)epitopes selected from SEQ ID NOs: 8, 22, 31, 32, 42, 77, and 97; or (h) CTL (neo)epitopes selected from SEQ ID NOs: 8, 22, 31, 32, 42, 52, and 97; (i) CTL (neo)epitopes selected from SEQ ID NOs: 8, 22, 31, 32, 52, 77 and 97;
CTL (neo)epitopes of SEQ ID NOs: 23, 66, 70 and 146 and (a) CTL (neo)epitopes selected from SEQ ID NOs: 8, 22, 31, 32, 42, 52, 77, and 97; (b) CTL (neo)epitopes selected from SEQ ID NOs: 8, 22, 31, 32, 42, 52 and 77; (c) CTL (neo)epitopes selected from SEQ ID NOs: 22, 31, 32, 42, 52, 77, 9 and 7; (d) CTL (neo)epitopes selected from SEQ ID NOs: 8, 31, 32, 42, 52, 77 and 97; (e) CTL (neo)epitopes selected from SEQ ID NOs: 8, 22, 32, 42, 52, 77 and 97; (f) CTL (neo)epitopes selected from SEQ ID NOs: 8, 22, 31, 42, 52, 77 and 97; (g) CTL (neo)epitopes selected from SEQ ID NOs: 8, 22, 31, 32, 42, 77 and 97; or (h) CTL (neo)epitopes selected from SEQ ID NOs: 8, 22, 31, 32, 42, 52 and 97; (i) CTL (neo)epitopes selected from SEQ ID NOs: 8, 22, 31, 32, 52, 77 and 97.

In a particular aspect, the vaccine composition comprises at least one (neo)epitope inducing a virus-specific tissue-resident memory T lymphocytes (Trm). Accordingly, the vaccine composition preferably comprises at least 1, 2, 3, 4, 5 or 6 CTL (neo)epitopes selected in one of the groups consisting of (i) SEQ ID NOs: 20, 23, 32, 36, 42, 56, 59, 60, 76, 79, 85, 91, 95, 97, 125, 140 and 146; (ii) SEQ ID NOs: 23, 32, 36, 42, 56, 59, 60, 76, 79, 91, 97, 125, 140 and 146; and (iii) SEQ ID NOs: 23, 32, 42, 97 and 146.

The addition of a specific HTL epitope as a provider of CD4+ T Helper Lymphocytes cells is an important factor in the combination of (neo)epitopes with T Helper support for the global immune response, both in the B cell early responses and in the T cell Long term specific memory responses.

Considering COVID 19, it has been reported that CD4+ T cell responses correlated with positive outcomes (Braun et al, medRxiv 2020 Presence of SARS-CoV-2-reactive T cells in COVID-19 patients and healthy donors).

The HTL epitopes are either natural or synthetic T cells helper peptides know in the art. Natural helper peptides are for instance a Natural Tetanus sequence alone or linked to another epitope, or a Plasmodium falciparum sequence alone or linked to another epitope.

In a particular aspect, the HTL peptide may comprise a synthetic peptide such as a Pan-DR-binding epitope (e.g., a PADRE® peptide, Epimmune Inc., San Diego, Calif., described, for example, in U.S. Pat. No. 5,736,142), for example, having the formula aKXVAAZTLKAAa, where “X” is either cyclohexylalanine, phenylalanine, or tyrosine; “Z” is either tryptophan, tyrosine, histidine or asparagine; and “a” is either D-alanine or L-alanine (SEQ ID NO: 746). Certain pan-DR binding epitopes comprise all “L” natural amino acid residues; these molecules can be provided as peptides or in the form of nucleic acids that encode the peptide. See also, U.S. Pat. Nos. 5,679,640 and 6,413,935.

The vaccine composition may comprise adjuvants. The adjuvant is preferably an oily adjuvant, which comprises both a hydrocarbon oil and a water-in-oil emulsifier. Such adjuvants act by the so-called “deposition effect”. The hydrocarbon oil may be paraffin oil, a vegetable oil, squalene, squalane or mineral oil, for instance. Suitable W/O emulsifiers may be selected from mannide mono-oleate and sorbitan mono-oleate, for instance. Examples of appropriate oily adjuvants are a mixture of 5-20% mannide mono-oleate with 80-95% mineral oil (Montanide® ISA 51 sold by SEPPIC) or squalene (Montanide® ISA 720 sold by SEPPIC) and similar mixtures. In a specific embodiment, the adjuvant is a mixture of mineral oil and mannide mono-oleate, especially Montanide® ISA 51.

In a particular aspect, the vaccine composition is an emulsion with a mineral oil adjuvant.

The adjuvant used in this invention may alternatively, or in addition to the above oily adjuvants, be selected from micro- and nanoparticles, such as liposomes and microspheres, of PLG, PLA, PLGA or other natural polymers such as gelatin, collagen and chitosan. Other adjuvants may comprise TLR ligands, Toll-like receptor ligands (TLR3 and TLR9), stimulators of IFN genes (STING) agonists, cytokines such as GM-CSF and IL2, carbohydrates, bacterial derivatives, mineral salts and immune stimulating complexes (ISCOM).

Optionally, the vaccine composition may comprise aluminum salts, such as aluminum hydroxide, aluminum phosphate, and aluminum potassium sulfate.

The vaccine compositions are intended for parenteral, topical, oral, intrathecal, or local administration. Preferably, the vaccine compositions are administered parentally, e.g., intravenously, subcutaneously, intradermally, or intramuscularly. More preferably, the vaccine composition is intended for subcutaneous administration or intramuscular administration. Optionally, the vaccine composition is intended for nasal administration.

Optionally, each peptide of the composition is present at a concentration of 0.01 mg/ml to 1 g/ml, 0.1 mg/ml to 10 mg/ml. For instance, each peptide can be present at a concentration of 0.5 mg/ml.

In a particular aspect, the vaccine composition is to be administered once, twice or more. For instance, two administrations can be carried out. A prime administration followed by a boost administration. For instance, the injections can be spaced by 3 weeks or 2 weeks and will be adapted to the Immune response requested and to the medical conditions of the subject to be treated.

The present invention relates to a composition of the present invention for use for preventing or treating an infection by a severe acute respiratory syndrome-related coronavirus (SARS-CoV), the use of a composition of the present invention for the manufacture of a vaccine for preventing or treating an infection by a severe acute respiratory syndrome-related coronavirus (SARS-CoV), and to a method for preventing or treating an infection by a severe acute respiratory syndrome-related coronavirus (SARS-CoV) in a subject, comprising the administration of an effective amount of a composition of the present invention.

Optionally, the SARS-CoV is selected from the group consisting of SARS-CoV1, SARS-CoV2 or MERS-CoV virus. Optionally, the SARS-CoV is SARS-CoV1. Optionally, the SARS-CoV is SARS-CoV2. Optionally, the SARS-CoV is MERS-CoV virus.

The present invention relates to a composition of the present invention for use for preventing or treating Covid-19, the use of a composition of the present invention for the manufacture of a vaccine for preventing or treating Covid-19, and to a method for preventing or treating an infection by Covid-19 in a subject, comprising the administration of an effective amount of a composition of the present invention.

Optionally, the subject to be treated is a subject aged 65 years or older, a subject having a cancer or having had a cancer, a subject being obese (In particular with severe obesity (body mass index [BMI] of 40 or higher [CDC-HCSP BMI>30]), a subject being diabetic, a subject having a hypertension, a subject having a sarcoidosis, a subject being immunocompromised, a subject who lives in a nursing home or long-term care facility, a subject with chronic lung disease or moderate to severe asthma, lung fibrosis, a subject who has serious heart conditions, a subject with chronic kidney disease undergoing dialysis and/or a subject with liver diseases. Optionally, the subject can be a subject with a stable comorbidity factor, for instance, stable cancer patients, chronic obstructive pulmonary disease (COPD) patients, stable patients with comorbidity as Obesity or renal dialysis (10 volunteers planned by group of comorbidity).

Many conditions can cause a person to be immunocompromised (including cancer treatment, smoking, bone marrow or organ transplantation, immune deficiencies, poorly controlled HIV or AIDS, prolonged use of corticosteroids and other immune weakening medications).

The patient could be selected on HLA typing. In one aspect, the subject to be treated has one of the HLA typing disclosed in any of the Tables 1-3. In a very specific aspect, the subject is HLA-A2.

However, the vaccine composition comprises a combination of peptides allowing the treatment of subjects having a broad diversity of HLA, then being suitable for the treatment of the worldwide population.

### Definitions

A “diluent” includes sterile liquids, such as water and oils, including those of petroleum, animal, vegetable or synthetic origin, such as peanut oil, soybean oil, mineral oil, sesame oil and the like. Water is a preferred diluent for pharmaceutical compositions. Saline solutions and aqueous dextrose and glycerol solutions can also be employed as diluents, particularly for injectable solutions.

An “epitope” is the collective features of a molecule, such as primary, secondary and tertiary peptide structure, and charge, that together form a site recognized by an immunoglobulin, T cell receptor or HLA molecule. Alternatively, an epitope can be defined as a set of amino acid. residues which is involved in recognition by a particular immunoglobulin, or in the context of T cells, those residues necessary for recognition by T cell receptor proteins and/or Major Histocompatibility Complex (MHC) receptors. Epitopes are present in nature, and can be isolated, purified or otherwise prepared or derived by humans. For example, epitopes can be prepared by isolation from a natural source, or they can be synthesized in accordance with standard protocols in the art. Synthetic epitopes can comprise artificial amino acid residues, “amino acid mimetics,” such as D isomers of naturally-occurring L amino acid residues or non-naturally-occurring amino acid residues such as cyclohexylalanine. Throughout this disclosure, epitopes may be referred to in some cases as peptides or peptide epitopes.

“Human Leukocyte Antigen” or “HLA” is a human class I or class II Major Histocompatibility Complex (MHC) protein (see, e.g., Stites, et al., IMMUNOLOGY, 8TH ED., Lange Publishing, Los Altos, Calif. (1994).

An “HLA supertype or HLA family”, as used herein, describes sets of HLA molecules grouped on the basis of shared peptide-binding specificities. HLA class I molecules that share somewhat similar binding affinity for peptides bearing certain amino acid motifs are grouped into such HLA supertypes. The terms HLA superfamily, HLA supertype family, HLA family, and HLA xx-like molecules (where “xx” denotes a particular HLA type), are synonyms.

“Major Histocompatibility Complex” or “MHC” is a cluster of genes that plays a role in control of the cellular interactions responsible for physiologic immune responses. In humans, the MHC complex is also known as the human leukocyte antigen (HLA) complex. For a detailed description of the MHC and HLA complexes, see, Paul, FUNDAMENTAL IMMUNOLOGY, 3RD ED., Raven Press, New York (1993).

A “native” or a “wild type” sequence refers to a sequence found in nature. Such a sequence may comprise a longer sequence in nature.

The terms “peptide”, “epitope” and “peptide epitope” are used interchangeably with “oligopeptide” in the present specification to designate a series of residues, typically L-amino acid residues, connected one to the other, typically by peptide bonds between the α-amino and carboxyl groups of adjacent amino acid residues.

Optionally, a “peptide”, “epitope” and “peptide epitope” defined by a SEQ ID NO can consist in the particular SEQ ID NO and can also refer to a peptide consisting in the particular SEQ ID NO but including 1 or 2 additional amino acids at the N and/or C terminal end of the SEQ ID NO.

Optionally, one or several “peptide”, “epitope” and “peptide epitope” can be fused together in a same polypeptide.

A “PanDR binding” peptide, a “PanDR binding epitope,” or “PADRE®” peptide (Epimmune, San Diego, Calif.) is a member of a family of molecules that binds more than one HLA class II DR molecule. The pattern that defines the PADRE® family of molecules can be referred to as an HLA Class II supermotif. A PADRE® molecule binds to HLA-DR molecules and stimulates in vitro and in vivo human helper T lymphocyte (HTL) responses. For a further definition of the PADRE® family, see copending application U.S. Ser. No. 09/709,774, filed Nov. 11, 2000; and Ser. No. 09/707,738, filed Nov. 6, 2000; PCT publication Nos WO 95/07707, and WO 97/26784; U.S. Pat. No. 5,736,142 issued Apr. 7, 1998; U.S. Pat. No. 5,679,640, issued Oct. 21, 1997; and U.S. Pat. No. 6,413,935, issued Jul. 2, 2002.

“Pharmaceutically acceptable” refers to a generally non-toxic, inert, and/or physiologically compatible composition or component of a composition.

A “pharmaceutical excipient” or “excipient” comprises a material such as an adjuvant, a carrier, pH-adjusting and buffering agents, tonicity adjusting agents, wetting agents, preservatives, and the like. A “pharmaceutical excipient” is an excipient which is pharmaceutically acceptable.

A “protective immune response” or “therapeutic immune response” refers to a BCL, CTL and/or an HTL response to an antigen derived from a pathogenic antigen (e.g., an antigen from an infectious agent or a tumor antigen), which in some way prevents or at least partially arrests disease symptoms, side effects or progression. The immune response may also include an antibody response which has been facilitated by the stimulation of helper T cells.

As used herein, a “vaccine” is a composition used for vaccination, e.g., for prophylaxis or therapy, that comprises one or more peptides of the invention. There are numerous embodiments of vaccines in accordance with the invention, such as by a cocktail of one or more peptides; one or more peptides of the invention comprised by a polyepitopic peptide; or nucleic acids that encode such peptides or polypeptides, e.g., a minigene that encodes a polyepitopic peptide. The “one or more peptides” can include any whole unit integer from 1-50, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 peptides of the invention. The peptides or polypeptides can optionally be modified, such as by lipidation, addition of targeting or other sequences. HLA class I-binding peptides of the invention can be linked or to otherwise be combined with HLA class II-binding peptides, e.g., a PADRE® universal HTL-binding peptide, to facilitate activation of both cytotoxic T lymphocytes and helper T lymphocytes. Vaccines can comprise peptide pulsed antigen presenting cells, e.g., dendritic cells.

TABLE 1
wild-type epitope; CTL epitopes

| | SEQ | | | |
|---|---|---|---|---|
| Pep | ID | Target | | |
| ID | NO | protein | Sequence | HLA binding |
| 1_wt | 1 | Spike | ALNTLVKQL | HLA-A*02:01; HLA-A*32:01; HLA-B*13:01; HLA- |
| | | glycoprotein | | B*48:01; HLA-C*02:02; HLA-C*02:09; HLA- |
| | | | | C*07:04; HLA-C*17:01; HLA-E*01:01 |
| 2_wt | 2 | N protein | ALNTPKDHI | HLA-A*02:01; HLA-A*02:02 |
| 3_wt | 3 | Protein 3a | ALSKGVHFV | HLA-A*02:01; HLA-A*02:06; HLA-A*32:01; HLA- |
| | | | | C*02:02; HLA-C*02:09; HLA-C*05:01; HLA- |
| | | | | C*07:04; HLA-C*08:02; HLA-C*15:02; HLA- |
| | | | | C*17:01; HLA-E*01:01 |
| 4_wt | 4 | ORF1ab | AMYTPHTVL | HLA-A*02:01; HLA-A*02:06; HLA-A*30:01; HLA- |
| | | (nsp12) | | A*32:01; HLA-B*07:02; HLA-B*08:01; HLA- |
| | | | | B*13:01; HLA-B*13:02; HLA-B*14:02; HLA- |
| | | | | B*15:01; HLA-B*15:02; HLA-B*15:25; HLA- |
| | | | | B*37:01; HLA-B*38:01; HLA-B*39:01; HLA- |
| | | | | B*46:01; HLA-B*48:01; HLA-B*52:01; HLA- |
| | | | | B*55:01; HLA-B*58:02; HLA-C*01:02; HLA- |
| | | | | C*02:02; HLA-C*02:09; HLA-C*03:02; HLA- |
| | | | | C*03:03; HLA-C*03:04; HLA-C*04:01; HLA- |
| | | | | C*05:01; HLA-C*06:02; HLA-C*07:01; HLA- |
| | | | | C*07:02; HLA-C*07:04; HLA-C*08:01; HLA- |
| | | | | C*08:02; HLA-C*12:02; HLA-C*12:03; HLA- |
| | | | | C*14:02; HLA-C*15:02; HLA-C*16:01; HLA- |
| | | | | C*17:01; HLA-E*01:01; HLA-G*01:02; HLA- |
| | | | | G*01:03; HLA-G*01:04; HLA-G*01:06 |
| 5_wt | 5 | ORF1ab | CLEASFNYL | HLA-A*02:01; HLA-A*02:02 |
| | | (nsp3) | | |
| 6_wt | 6 | Spike | FIAGLIAIV | HLA-A*02:01; HLA-A*02:06; HLA-A*25:01; HLA- |
| | | glycoprotein | | A*26:01; HLA-A*68:02; HLA-C*01:02; HLA- |
| | | | | C*02:02; HLA-C*02:09; HLA-C*03:03; HLA- |
| | | | | C*03:04; HLA-C*05:01; HLA-C*08:01; HLA- |
| | | | | C*08:02; HLA-C*12:02; HLA-C*12:03; HLA- |
| | | | | C*15:02; HLA-C*17:01 |
| 7_wt | 7 | ORF1Ab | FLAHIQWMV | HLA-A*02:01; HLA-A*02:06; HLA-A*68:02; HLA- |
| | | (nsp4) | | C*04:01; HLA-C*05:01; HLA-C*08:01; HLA- |
| | | | | C*08:02; HLA-C*17:01 |
| 8_wt | 8 | ORF1ab | FLLNKEMYL | HLA-A*02:01; HLA-A*02:06; HLA-A*32:01; HLA- |
| | | (nsp4) | | B*08:01; HLA-B*14:02; HLA-B*46:01; HLA- |
| | | | | B*48:01; HLA-C*01:02; HLA-C*02:02; HLA- |
| | | | | C*02:09; HLA-C*03:02; HLA-C*03:03; HLA- |
| | | | | C*03:04; HLA-C*04:01; HLA-C*05:01; HLA- |
| | | | | C*06:02; HLA-C*07:01; HLA-C*07:02; HLA- |
| | | | | C*07:04; HLA-C*08:01; HLA-C*08:02; HLA- |
| | | | | C*12:02; HLA-C*12:03; HLA-C*14:02; HLA- |
| | | | | C*15:02; HLA-C*16:01; HLA-C*17:01; HLA- |
| | | | | E*01:01; HLA-G*01:02; HLA-G*01:03; HLA- |
| | | | | G*01:04; HLA-G*01:06 |
| 9_wt | 9 | ORF1ab | FLTENLLLY | HLA-A*01:01; HLA-A*25:01; HLA-A*26:01; HLA- |
| | | (nsp3) | | A*29:02; HLA-A*30:02; HLA-A*32:01; HLA- |
| | | | | B*15:01; HLA-B*15:02; HLA-B*15:25; HLA- |
| | | | | B*35:01; HLA-B*46:01; HLA-B*53:01; HLA- |
| | | | | C*02:02; HLA-C*02:09; HLA-C*03:02; HLA- |
| | | | | C*04:01; HLA-C*05:01; HLA-C*06:02; HLA- |
| | | | | C*07:01; HLA-C*07:02; HLA-C*08:01; HLA- |
| | | | | C*08:02; HLA-C*12:02; HLA-C*12:03; HLA- |
| | | | | C*14:02; HLA-C*16:01; HLA-C*17:01 |
| 10_wt | 10 | Spike | FQFCNDPFL | HLA-A*02:01; HLA-A*02:06; HLA-B*13:01; HLA- |
| | | glycoprotein | | B*13:02; HLA-B*38:01; HLA-B*39:01; HLA- |
| | | | | B*48:01; HLA-B*52:01; HLA-C*03:02; HLA- |
| | | | | C*03:03; HLA-C*03:04; HLA-C*08:01; HLA- |
| | | | | C*08:02; HLA-C*12:02; HLA-C*17:01; HLA-E*01:01 |
| 11_wt | 11 | membrane | FVLAAVYRI | HLA-A*02:01; HLA-A*02:06; HLA-A*32:01; HLA- |
| | | glycoP | | A*68:02; HLA-B*13:02; HLA-B*51:01; HLA- |
| | | | | B*52:01; HLA-C*01:02; HLA-C*02:02; HLA- |
| | | | | C*02:09; HLA-C*03:02; HLA-C*03:03; HLA- |
| | | | | C*03:04; HLA-C*05:01; HLA-C*07:04; HLA- |
| | | | | C*08:01; HLA-C*08:02; HLA-C*12:02; HLA- |
| | | | | C*12:03; HLA-C*15:02; HLA-C*16:01; HLA-C*17:01 |
| 12_wt | 12 | Spike | GKQGNFKNL | HLA-A*02:01; HLA-A*02:02 |
| | | glycoprotein | | |
| 13_wt | 13 | ORF1 | GLFKDCSKV | HLA-A*02:01; HLA-A*02:06 |
| | | (nsp14) | | |
| 14_wt | 14 | M protein | GLMWLSYFI | HLA-A*02:01; HLA-A*02:06 |
| 15_wt | 15 | N protein | GMSRIGMEV | HLA-A*02:06; HLA-A*32:01; HLA-C*17:01 |
| 16_wt | 16 | Spike | GYQPYRVVVL | HLA-A*23:01; HLA-A*24:02; HLA-C*04:01; HLA- |
| | | glycoprotein | | C*06:02; HLA-C*07:01; HLA-C*07:02; HLA- |
| | | | | C*07:04; HLA-C*14:02; HLA-G*01:02; HLA- |
| | | | | G*01:03; HLA-G*01:04; HLA-G*01:06 |
| 17_wt | 17 | ORF1ab (nsp3) | ILLLDQALV | HLA-A*02:01; HLA-A*02:06; HLA-C*17:01 |
| 18_wt | 18 | N protein | ILLNKHIDA | HLA-A*02:01; HLA-A*02:02 |
| 19_wt | 19 | Spike | KIADYNYKL | HLA-A*02:01; HLA-A*02:06; HLA-A*32:01; HLA- |
| | | glycoprotein | | A*68:02; HLA-A*74:01; HLA-B*13:01; HLA- |
| | | | | B*15:25; HLA-B*46:01; HLA-B*48:01; HLA- |
| | | | | B*57:01; HLA-B*58:01; HLA-B*58:02; HLA- |
| | | | | C*01:02; HLA-C*02:02; HLA-C*02:09; HLA- |
| | | | | C*03:02; HLA-C*03:03; HLA-C*03:04; HLA- |
| | | | | C*04:01; HLA-C*05:01; HLA-C*06:02; HLA- |
| | | | | C*07:01; HLA-C*07:02; HLA-C*07:04; HLA- |
| | | | | C*08:01; HLA-C*08:02; HLA-C*12:02; HLA- |
| | | | | C*12:03; HLA-C*14:02; HLA-C*15:02; HLA- |
| | | | | C*16:01; HLA-C*17:01; HLA-E*01:01; HLA- |
| | | | | G*01:02; HLA-G*01:03; HLA-G*01:04; HLA- |
| | | | | G*01:06 |
| 20_wt | 20 | ORF1ab | KLKDCVMYA | HLA-A*02:01; HLA-A*02:06; HLA-A*30:01 |
| | | (nsp6) | | |
| 21_wt | 21 | Spike | KLPDDFTGCV | HLA-A*02:01; HLA-A*02:06 |
| | | glycoprotein | | |
| 22_wt | 22 | ORFA1b | KLSYGIATV | HLA-A*02:01; HLA-A*02:06; HLA-A*32:01; HLA- |
| | | (nsp13) | | C*01:02; HLA-C*02:02; HLA-C*02:09; HLA- |
| | | | | C*05:01; HLA-C*08:01; HLA-C*08:02; HLA- |
| | | | | C*12:02; HLA-C*15:02; HLA-C*16:01; HLA- |
| | | | | C*17:01; HLA-E*01:01 |
| 23_wt | 23 | N protein | LALLLLDRL | HLA-B*08:01; HLA-C*01:02; HLA-C*03:03; HLA- |
| | | | | C*03:04; HLA-C*12:02; HLA-C*12:03; HLA- |
| | | | | C*15:02; HLA-C*16:01; HLA-E*01:01 |
| 24_wt | 24 | Spike | LITGRLQSL | HLA-A*02:01; HLA-A*02:06; HLA-A*32:01; HLA- |
| | | glycoprotein | | B*08:01; HLA-B*14:02; HLA-B*46:01; HLA- |
| | | | | B*48:01; HLA-C*01:02; HLA-C*02:02; HLA- |
| | | | | C*02:09; HLA-C*03:02; HLA-C*03:03; HLA- |
| | | | | C*03:04; HLA-C*04:01; HLA-C*05:01; HLA- |
| | | | | C*06:02; HLA-C*07:01; HLA-C*07:02; HLA- |
| | | | | C*07:04; HLA-C*08:01; HLA-C*08:02; HLA- |
| | | | | C*12:02; HLA-C*12:03; HLA-C*14:02; HLA- |
| | | | | C*15:02; HLA-C*16:01; HLA-C*17:01; HLA- |
| | | | | E*01:01; HLA-G*01:02; HLA-G*01:03; HLA- |
| | | | | G*01:04; HLA-G*01:06 |
| 25_wt | 25 | N protein | LLLDRLNQL | HLA-A*02:01; HLA-A*02:06; HLA-A*32:01; HLA- |
| | | | | B*48:01; HLA-C*01:02; HLA-C*03:03; HLA- |
| | | | | C*03:04; HLA-C*08:01; HLA-C*17:01 |
| 26_wt | 26 | ORF1ab | LLLTILTSL | HLA-A*02:01; HLA-A*02:06 |
| | | (nsp6) | | |
| 27_wt | 27 | ORF1Ab | LLSAGIFGA | HLA-A*02:01; HLA-A*02:02 |
| | | (nsp3) | | |
| 28_wt | 28 | ORF1Ab | LMWLIINLV | HLA-A*02:06; HLA-A*32:01; HLA-B*13:01; HLA- |
| | | (nsp3) | | B*13:02; HLA-B*14:02; HLA-B*15:01; HLA- |
| | | | | B*15:02; HLA-B*15:25; HLA-B*37:01; HLA- |
| | | | | B*38:01; HLA-B*39:01; HLA-B*40:01; HLA- |
| | | | | B*40:02; HLA-B*46:01; HLA-B*48:01; HLA- |
| | | | | B*49:01; HLA-B*50:01; HLA-B*52:01; HLA- |
| | | | | C*01:02; HLA-C*02:02; HLA-C*02:09; HLA- |
| | | | | C*03:02; HLA-C*03:03; HLA-C*03:04; HLA- |
| | | | | C*04:01; HLA-C*06:02; HLA-C*07:01; HLA- |
| | | | | C*07:02; HLA-C*07:04; HLA-C*08:01; HLA- |
| | | | | C*08:02; HLA-C*12:02; HLA-C*12:03; HLA- |
| | | | | C*14:02; HLA-C*15:02; HLA-C*16:01; HLA- |
| | | | | C*17:01; HLA-E*01:01 |
| 29_wt | 29 | N protein | LQLPQGTTL | HLA-A*02:01; HLA-A*02:06; HLA-A*68:02; HLA- |
| | | | | C*02:02; HLA-C*02:09; HLA-C*15:02; HLA-C*17:01 |
| 30_wt | 30 | ORF1Ab | MLAKALRKV | HLA-A*02:01; HLA-A*02:06; HLA-A*23:01; HLA- |
| | | (nsp3) | | A*24:02; HLA-A*25:01; HLA-A*29:02; HLA- |
| | | | | A*32:01; HLA-A*68:02; HLA-B*08:01; HLA- |
| | | | | B*13:01; HLA-B*13:02; HLA-B*14:02; HLA- |
| | | | | B*15:01; HLA-B*15:02; HLA-B*15:25; HLA- |
| | | | | B*35:01; HLA-B*35:03; HLA-B*38:01; HLA- |
| | | | | B*39:01; HLA-B*46:01; HLA-B*48:01; HLA- |
| | | | | B*52:01; HLA-B*56:01; HLA-C*01:02; HLA- |
| | | | | C*02:02; HLA-C*02:09; HLA-C*03:02; HLA- |
| | | | | C*03:03; HLA-C*03:04; HLA-C*04:01; HLA- |
| | | | | C*05:01; HLA-C*07:01; HLA-C*07:02; HLA- |
| | | | | C*07:04; HLA-C*08:01; HLA-C*08:02; HLA- |
| | | | | C*12:02; HLA-C*12:03; HLA-C*14:02; HLA- |
| | | | | C*15:02; HLA-C*16:01; HLA-C*17:01; HLA- |
| | | | | E*01:01; HLA-G*01:02; HLA-G*01:03; HLA- |
| | | | | G*01:04; HLA-G*01:06 |
| 31_wt | 31 | ORF1Ab | MMISAGFSL | HLA-A*02:01; HLA-A*02:06; HLA-A*68:02; HLA- |
| | | (nsp14) | | B*13:02; HLA-B*52:01; HLA-C*05:01; HLA- |
| | | | | C*07:04; HLA-C*08:01; HLA-C*08:02; HLA-C*17:01 |
| 32_wt | 32 | ORF1ab | NLIDSYFVV | HLA-A*02:01; HLA-A*02:06 |
| | | (nsp12) | | |
| 33_wt | 33 | ORF1ab | NLLKDCPAV | HLA-A*02:01; HLA-A*02:06; HLA-C*05:01; HLA- |
| | | (nsp12) | | C*08:01; HLA-C*08:02; HLA-C*17:01 |
| 34_wt | 34 | Spike | NLNESLIDL | HLA-A*02:01; HLA-A*02:02 |
| | | glycoprotein | | |
| 35_wt | 35 | Spike | PYRWVLSF | HLA-A*02:01; HLA-A*02:02 |
| | | glycoprotein | | |
| 36_wt | 36 | ORF1ab | QLEMELTPV | HLA-A*02:02; HLA-A*23:01; HLA-A*24:02; HLA- |
| | | (nsp3) | | C*14:02 |
| 37_wt | 37 | ORF1ab | RIMTWLDMV | HLA-A*02:01; HLA-A*02:06; HLA-A*32:01; HLA- |
| | | (nsp6) | | C*15:02; HLA-C*17:01 |
| 38_wt | 38 | Spike | RLNEVAKNL | HLA-A*02:01; HLA-A*02:06; HLA-A*32:01; HLA- |
| | | glycoprotein | | B*13:01; HLA-B*13:02; HLA-B*15:01; HLA- |
| | | | | B*15:25; HLA-B*46:01; HLA-B*48:01; HLA- |
| | | | | B*58:02; HLA-C*01:02; HLA-C*02:02; HLA- |
| | | | | C*02:09; HLA-C*03:02; HLA-C*03:03; HLA- |
| | | | | C*03:04; HLA-C*04:01; HLA-C*05:01; HLA- |
| | | | | C*06:02; HLA-C*07:01; HLA-C*07:02; HLA- |
| | | | | C*07:04; HLA-C*08:01; HLA-C*08:02; HLA- |
| | | | | C*12:02; HLA-C*12:03; HLA-C*14:02; HLA- |
| | | | | C*15:02; HLA-C*16:01; HLA-C*17:01; HLA- |
| | | | | E*01:01; HLA-G*01:02; HLA-G*01:03; HLA- |
| | | | | G*01:04; HLA-G*01:06 |
| 39_wt | 39 | N protein | RLNQLESKM | HLA-A*32:01; HLA-B*15:01; HLA-B*15:25; HLA- |
| | | | | B*46:01; HLA-C*02:02; HLA-C*02:09; HLA- |
| | | | | C*03:02; HLA-C*12:02; HLA-C*15:02; HLA-C*17:01 |
| 40_wt | 40 | Spike | SIIAYTMSL | HLA-A*02:01; HLA-A*02:06; HLA-A*25:01; HLA- |
| | | glycoprotein | | A*26:01; HLA-A*32:01; HLA-A*68:02; HLA- |
| | | | | B*07:02; HLA-B*08:01; HLA-B*13:01; HLA- |
| | | | | B*14:02; HLA-B*15:02; HLA-B*15:25; HLA- |
| | | | | B*35:03; HLA-B*39:01; HLA-B*46:01; HLA- |
| | | | | B*48:01; HLA-B*52:01; HLA-B*55:01; HLA- |
| | | | | B*56:01; HLA-C*01:02; HLA-C*02:02; HLA- |
| | | | | C*02:09; HLA-C*03:02; HLA-C*03:03; HLA- |
| | | | | C*03:04; HLA-C*04:01; HLA-C*05:01; HLA- |
| | | | | C*06:02; HLA-C*07:01; HLA-C*07:02; HLA- |
| | | | | C*07:04; HLA-C*08:01; HLA-C*08:02; HLA- |
| | | | | C*12:02; HLA-C*12:03; HLA-C*14:02; HLA- |
| | | | | C*15:02; HLA-C*16:01; HLA-C*17:01; HLA- |
| | | | | E*01:01; HLA-G*01:02; HLA-G*01:03; HLA- |
| | | | | G*01:04; HLA-G*01:06 |
| 41_wt | 41 | ORF1ab | SLPGVFCGV | HLA-A*02:01; HLA-A*02:06; HLA-A*68:02; HLA- |
| | | (nsp4) | | C*17:01; HLA-E*01:01 |
| 42_wt | 42 | ORF1ab | SMWALIISV | HLA-A*02:01; HLA-A*02:06; HLA-A*32:01; HLA- |
| | | (nsp6) | | B*13:02; HLA-B*48:01; HLA-B*52:01; HLA- |
| | | | | C*08:01; HLA-C*17:01 |
| 43_wt | 43 | M protein | TLACFVLAAV | HLA-A*02:01; HLA-A*02:02 |
| 44_wt | 44 | ORF1ab | TLMNVLTLV | HLA-A*02:01; HLA-A*02:06; HLA-C*17:01; HLA- |
| | | (nsp6) | | A*68:02; HLA-C*02:02; HLA-C*02:09; HLA- |
| | | | | C*15:02; HLA-C*05:01; HLA-A*32:01; HLA- |
| | | | | B*13:02; HLA-C*07:04; HLA-C*08:01 |
| 45_wt | 45 | Spike | VLNDILSRL | HLA-A*02:01; HLA-A*02:06; HLA-A*25:01; HLA- |
| | | glycoprotein | | A*32:01; HLA-A*68:02; HLA-B*13:01; HLA- |
| | | | | B*13:02; HLA-B*15:01; HLA-B*15:25; HLA- |
| | | | | B*46:01; HLA-B*48:01; HLA-C*01:02; HLA- |
| | | | | C*02:02; HLA-C*02:09; HLA-C*03:02; HLA- |
| | | | | C*03:03; HLA-C*03:04; HLA-C*04:01; HLA- |
| | | | | C*05:01; HLA-C*06:02; HLA-C*07:01; HLA- |
| | | | | C*07:02; HLA-C*07:04; HLA-C*08:01; HLA- |
| | | | | C*08:02; HLA-C*12:02; HLA-C*12:03; HLA- |
| | | | | C*14:02; HLA-C*15:02; HLA-C*16:01; HLA- |
| | | | | C*17:01; HLA-E*01:01; HLA-G*01:02; HLA- |
| | | | | G*01:03; HLA-G*01:04; HLA-G*01:06 |
| 46_wt | 46 | ORF1Ab | VQMAPISAM | HLA-A*02:06; HLA-A*25:01; HLA-A*26:01; HLA- |
| | | (nsp3) | | A*32:01; HLA-B*07:02; HLA-B*13:01; HLA- |
| | | | | B*13:02; HLA-B*14:02; HLA-B*15:01; HLA- |
| | | | | B*15:02; HLA-B*15:25; HLA-B*18:01; HLA- |
| | | | | B*35:01; HLA-B*35:03; HLA-B*37:01; HLA- |
| | | | | B*38:01; HLA-B*39:01; HLA-B*40:01; HLA- |
| | | | | B*40:02; HLA-B*46:01; HLA-B*48:01; HLA- |
| | | | | B*49:01; HLA-B*50:01; HLA-B*52:01; HLA- |
| | | | | B*55:01; HLA-B*56:01; HLA-C*01:02; HLA- |
| | | | | C*02:02; HLA-C*02:09; HLA-C*03:02; HLA- |
| | | | | C*03:03; HLA-C*03:04; HLA-C*04:01; HLA- |
| | | | | C*06:02; HLA-C*07:01; HLA-C*07:02; HLA- |
| | | | | C*07:04; HLA-C*08:01; HLA-C*08:02; HLA- |
| | | | | C*12:02; HLA-C*12:03; HLA-C*14:02; HLA- |
| | | | | C*15:02; HLA-C*16:01; HLA-C*17:01; HLA- |
| | | | | E*01:01; HLA-G*01:02; HLA-G*01:03; HLA- |
| | | | | G*01:04; HLA-G*01:06 |
| 47_wt | 47 | Spike | VSPTKLNDL | HLA-C*01:02; HLA-E*01:01; HLA-G*01:02; HLA- |
| | | glycoprotein | | G*01:03; HLA-G*01:04; HLA-G*01:06 |
| 48_wt | 48 | Spike | VVFLHVTYV | HLA-A*02:01; HLA-A*02:06; HLA-A*30:01; HLA- |
| | | glycoprotein | | A*32:01; HLA-A*68:02; HLA-B*13:02; HLA- |
| | | | | B*51:01; HLA-B*52:01; HLA-B*55:01; HLA- |
| | | | | B*56:01; HLA-C*01:02; HLA-C*02:02; HLA- |
| | | | | C*02:09; HLA-C*03:02; HLA-C*03:03; HLA- |
| | | | | C*03:04; HLA-C*05:01; HLA-C*06:02; HLA- |
| | | | | C*07:01; HLA-C*07:02; HLA-C*07:04; HLA- |
| | | | | C*08:01; HLA-C*08:02; HLA-C*12:02; HLA- |
| | | | | C*12:03; HLA-C*15:02; HLA-C*16:01; HLA- |
| | | | | C*17:01; HLA-E*01:01 |
| 49_wt | 49 | ORF1ab | WLMWLIINL | HLA-A*02:01; HLA-A*02:06 |
| | | (nsp3) | | |
| 50_wt | 50 | ORF1ab | YLD AYN M M1 | HLA-A*01:01; HLA-A*02:01; HLA-A*02:06; HLA- |
| | | (nsp14) | | A*32:01; HLA-A*68:02; HLA-B*13:01; HLA- |
| | | | | B*13:02; HLA-B*38:01; HLA-B*39:01; HLA- |
| | | | | B*48:01; HLA-B*52:01; HLA-C*01:02; HLA- |
| | | | | C*02:02; HLA-C*02:09; HLA-C*03:02; HLA- |
| | | | | C*03:03; HLA-C*03:04; HLA-C*04:01; HLA- |
| | | | | C*05:01; HLA-C*06:02; HLA-C*07:01; HLA- |
| | | | | C*07:02; HLA-C*07:04; HLA-C*08:01; HLA- |
| | | | | C*08:02; HLA-C*12:02; HLA-C*12:03; HLA- |
| | | | | C*14:02; HLA-C*15:02; HLA-C*16:01; HLA- |
| | | | | C*17:01; HLA-E*01:01; HLA-G*01:02; HLA- |
| | | | | G*01:03; HLA-G*01:04; HLA-G*01:06 |
| 51_wt | 51 | ORF1ab | YLNSTNVTI | HLA-A*02:01; HLA-A*02:06; HLA-A*32:01; HLA- |
| | | (nsp3) | | A*68:02; HLA-B*13:01; HLA-B*13:02; HLA- |
| | | | | B*14:02; HLA-B*15:02; HLA-B*15:25; HLA- |
| | | | | B*35:03; HLA-B*38:01; HLA-B*39:01; HLA- |
| | | | | B*46:01; HLA-B*48:01; HLA-B*52:01; HLA- |
| | | | | B*55:01; HLA-B*56:01; HLA-C*01:02; HLA- |
| | | | | C*02:02; HLA-C*02:09; HLA-C*03:02; HLA- |
| | | | | C*03:03; HLA-C*03:04; HLA-C*04:01; HLA- |
| | | | | C*05:01; HLA-C*06:02; HLA-C*07:01; HLA- |
| | | | | C*07:02; HLA-C*07:04; HLA-C*08:01; HLA- |
| | | | | C*08:02; HLA-C*12:02; HLA-C*12:03; HLA- |
| | | | | C*14:02; HLA-C*15:02; HLA-C*16:01; HLA- |
| | | | | C*17:01; HLA-E*01:01; HLA-G*01:02; HLA- |
| | | | | G*01:03; HLA-G*01:04; HLA-G*01:06 |
| 52_wt | 52 | ORF1ab | YLNTLTLAV | HLA-A*02:01; HLA-A*02:06; HLA-B*46:01; HLA- |
| | | (nsp16) | | C*01:02; HLA-C*02:02; HLA-C*02:09; HLA- |
| | | | | C*03:02; HLA-C*03:03; HLA-C*03:04; HLA- |
| | | | | C*04:01; HLA-C*05:01; HLA-C*07:04; HLA- |
| | | | | C*08:01; HLA-C*08:02; HLA-C*12:02; HLA- |
| | | | | C*12:03; HLA-C*14:02; HLA-C*15:02; HLA- |
| | | | | C*16:01; HLA-C*17:01 |
| 53_wt | 53 | Spike | YLQPRTFLL | HLA-A*01:01; HLA-A*02:01; HLA-A*02:06; HLA- |
| | | glycoprotein | | A*23:01; HLA-A*24:02; HLA-A*29:02; HLA- |
| | | | | A*32:01; HLA-A*68:02; HLA-B*08:01; HLA- |
| | | | | B*13:01; HLA-B*14:02; HLA-B*38:01; HLA- |
| | | | | B*39:01; HLA-B*46:01; HLA-B*48:01; HLA- |
| | | | | B*52:01; HLA-C*01:02; HLA-C*02:02; HLA- |
| | | | | C*02:09; HLA-C*03:02; HLA-C*03:03; HLA- |
| | | | | C*03:04; HLA-C*04:01; HLA-C*05:01; HLA- |
| | | | | C*06:02; HLA-C*07:01; HLA-C*07:02; HLA- |
| | | | | C*07:04; HLA-C*08:01; HLA-C*08:02; HLA- |
| | | | | C*12:02; HLA-C*12:03; HLA-C*14:02; HLA- |
| | | | | C*15:02; HLA-C*16:01; HLA-C*17:01; HLA- |
| | | | | E*01:01; HLA-G*01:02; HLA-G*01:03; HLA- |
| | | | | G*01:04; HLA-G*01:06 |
| 54_wt | 54 | Protein 3a | YLYALVYFL | HLA-A*02:01; HLA-A*02:06; HLA-A*32:01; HLA- |
| | | | | A*68:02; HLA-B*48:01; HLA-C*01:02; HLA- |
| | | | | C*02:02; HLA-C*02:09; HLA-C*03:03; HLA- |
| | | | | C*03:04; HLA-C*04:01; HLA-C*05:01; HLA- |
| | | | | C*06:02; HLA-C*07:01; HLA-C*07:02; HLA- |
| | | | | C*07:04; HLA-C*08:01; HLA-C*08:02; HLA- |
| | | | | C*12:02; HLA-C*12:03; HLA-C*15:02; HLA- |
| | | | | C*16:01; HLA-C*17:01; HLA-E*01:01; HLA- |
| | | | | G*01:02; HLA-G*01:03; HLA-G*01:04; HLA- |
| | | | | G*01:06 |
| 55_wt | 55 | ORF1ab | YTMADLVYA | HLA-A*02:01; HLA-A*02:06; HLA-A*26:01; HLA- |
| | | (nsp12) | | A*68:02; HLA-B*46:01; HLA-B*56:01; HLA- |
| | | | | C*02:02; HLA-C*02:09; HLA-C*03:02; HLA- |
| | | | | C*03:03; HLA-C*03:04; HLA-C*05:01; HLA- |
| | | | | C*08:01; HLA-C*08:02; HLA-C*12:02; HLA- |
| | | | | C*12:03; HLA-C*15:02; HLA-C*16:01; HLA-C*17:01 |

TABLE 2
neo-epitope group A: CTL neoepitope

| | SEQ | Target | | |
|---|---|---|---|---|
| PepID | ID NO | protein | Sequence | HLA binding |
| 1_neoA | 56 | Spike | ALNTLVKQV | HLA-A*02:01; HLA-A*02:06; HLA-C*17:01 |
| | | glycoprotein | | |
| 2_neoA | 57 | N protein | ALNTPKDHV | HLA-A*02:01 |
| 4_neoA | 58 | ORF1ab | ALYTPHTVV | HLA-A*02:01; HLA-A*02:06; HLA-A*32:01; |
| | | (nsp12) | | HLA-B*13:02; HLA-B*15:25; HLA-B*46:01; |
| | | | | HLA-B*48:01; HLA-B*52:01; HLA-B*55:01; |
| | | | | HLA-C*01:02; HLA-C*02:02; HLA-C*02:09; |
| | | | | HLA-C*03:02; HLA-C*03:03; HLA-C*03:04; |
| | | | | HLA-C*04:01; HLA-C*05:01; HLA-C*07:04; |
| | | | | HLA-C*08:01; HLA-C*08:02; HLA-C*12:02; |
| | | | | HLA-C*12:03; HLA-C*14:02; HLA-C*15:02; |
| | | | | HLA-C*16:01; HLA-C*17:01; HLA-E*01:01 |
| 5_neoA | 59 | ORF1ab (nsp3) | CLEASFNYV | HLA-A*02:01 |
| 6_neoA | 60 | Spike | FLAGLIAIV | HLA-A*02:01; HLA-A*02:06; HLA-A*68:02; |
| | | glycoprotein | | HLA-C*01:02; HLA-C*02:02; HLA-C*02:09; |
| | | | | HLA-C*03:03; HLA-C*03:04; HLA-C*05:01; |
| | | | | HLA-C*08:01; HLA-C*08:02; HLA-C*12:02; |
| | | | | HLA-C*12:03; HLA-C*15:02; HLA-C*17:01 |
| 8_neoA | 61 | ORF1ab (nsp4) | FLLNKEMYV | HLA-A*02:01; HLA-A*02:06; HLA-A*68:02; |
| | | | | HLA-B*08:01; HLA-C*01:02; HLA-C*02:02; |
| | | | | HLA-C*02:09; HLA-C*04:01; HLA-C*05:01; |
| | | | | HLA-C*06:02; HLA-C*07:01; HLA-C*07:02; |
| | | | | HLA-C*07:04; HLA-C*08:01; HLA-C*08:02; |
| | | | | HLA-C*12:02; HLA-C*12:03; HLA-C*15:02; |
| | | | | HLA-C*16:01; HLA-C*17:01; HLA-E*01:01 |
| | | ` | | |
| 9_neoA | 62 | ORF1ab | FLTENLLLV | HLA-A*02:01; HLA-A*02:06; HLA-A*68:02; |
| | | (nsp3) | | HLA-B*46:01; HLA-C*01:02; HLA-C*02:02; |
| | | | | HLA-C*02:09; HLA-C*03:03; HLA-C*03:04; |
| | | | | HLA-C*04:01; HLA-C*05:01; HLA-C*07:04; |
| | | | | HLA-C*08:01; HLA-C*08:02; HLA-C*12:02; |
| | | | | HLA-C*12:03; HLA-C*15:02; HLA-C*17:01 |
| 10_neoA | 63 | Spike | FLFCNDPFV | HLA-A*02:01; HLA-A*02:06; HLA-A*68:02; |
| | | glycoprotein | | HLA-C*01:02; HLA-C*05:01; HLA-C*08:01; |
| | | | | HLA-C*08:02; HLA-C*17:01 |
| 11_neoA | 64 | membrane | FLLAAVYRV | HLA-A*02:01; HLA-A*02:06; HLA-A*68:02; |
| | | glycoP | | HLA-C*02:02; HLA-C*02:09; HLA-C*04:01; |
| | | | | HLA-C*05:01; HLA-C*07:04; HLA-C*08:01; |
| | | | | HLA-C*08:02; HLA-C*15:02; HLA-C*17:01 |
| 12_neoA | 65 | Spike | GLQGNFKNV | HLA-A*02:01; HLA-A*02:06 |
| | | glycoprotein | | |
| 14_neoA | 66 | M protein | GLMWLSYFV | HLA-A*02:01; HLA-A*02:06 |
| 15_neoA | 67 | N protein | GLSRIGMEV | HLA-A*02:01; HLA-A*02:06; HLA-C*05:01; |
| | | | | HLA-C*17:01 |
| 16_neoA | 68 | Spike | GLQPYRVVV | HLA-A*02:01; HLA-A*02:06; HLA-A*32:01; |
| | | glycoprotein | | HLA-C*01:02; HLA-C*05:01; HLA-C*07:04; |
| | | | | HLA-C*15:02; HLA-C*17:01; HLA-E*01:01 |
| 18_neoA | 69 | N protein | ILLNKHIDV | HLA-A*02:01; HLA-A*02:06; HLA-C*07:04; |
| | | | | HLA-C*17:01 |
| 19_neoA | 70 | Spike | KLADYNYKV | HLA-A*02:01; HLA-A*02:06; HLA-A*32:01; |
| | | glycoprotein | | HLA-A*68:02; HLA-A*74:01; HLA-B*13:01; |
| | | | | HLA-B*13:02; HLA-B*48:01; HLA-C*01:02; |
| | | | | HLA-C*02:02; HLA-C*02:09; HLA-C*04:01; |
| | | | | HLA-C*05:01; HLA-C*06:02; HLA-C*07:01; |
| | | | | HLA-C*07:02; HLA-C*07:04; HLA-C*08:01; |
| | | | | HLA-C*08:02; HLA-C*12:02; HLA-C*15:02; |
| | | | | HLA-C*16:01; HLA-C*17:01; HLA-E*01:01; |
| | | | | HLA-G*01:02; HLA-G*01:03; HLA-G*01:04; |
| | | | | HLA-G*01:06 |
| 20_neoA | 71 | ORF1ab | KLKDCVMYV | HLA-A*02:01; HLA-A*02:06; HLA-A*30:01; |
| | | (nsp6) | | HLA-A*32:01; HLA-C*02:02; HLA-C*02:09; |
| | | | | HLA-C*05:01; HLA-C*07:04; HLA-C*15:02; |
| | | | | HLA-C*17:01 |
| 21_neoA | 72 | Spike | KLPDDFTGV | HLA-A*02:01; HLA-A*02:06; HLA-A*68:02; |
| | | glycoprotein | | HLA-B*46:01; HLA-C*01:02; HLA-C*02:02; |
| | | | | HLA-C*02:09; HLA-C*04:01; HLA-C*05:01; |
| | | | | HLA-C*07:04; HLA-C*08:01; HLA-C*08:02; |
| | | | | HLA-C*15:02; HLA-C*17:01; HLA-E*01:01; |
| | | | | HLA-G*01:02; HLA-G*01:03; HLA-G*01:04; |
| | | | | HLA-G*01:06 |
| 23_neoA | 73 | N protein | LLLLLLDRV | HLA-A*02:01; HLA-A*02:06 |
| 24_neoA | 74 | Spike | LLTGRLQSV | HLA-A*02:01; HLA-A*02:06; HLA-B*08:01; |
| | | glycoprotein | | HLA-C*17:01 |
| 25_neoA | 75 | N protein | LLLDRLNQV | HLA-A*02:01; HLA-A*02:06; HLA-B*08:01; |
| | | | | HLA-C*02:02; HLA-C*02:09; HLA-C*04:01; |
| | | | | HLA-C*05:01; HLA-C*06:02; HLA-C*07:04; |
| | | | | HLA-C*08:01; HLA-C*08:02; HLA-C*15:02; |
| | | | | HLA-C*17:01 |
| 26_neoA | 76 | ORF1ab | LLLTILTSV | HLA-A*02:01; HLA-A*02:06; HLA-C*17:01 |
| | | (nsp6) | | |
| 27_neoA | 77 | ORF1Ab | LLSAGIFGV | HLA-A*02:01; HLA-A*02:06; HLA-A*68:02; |
| | | (nsp3) | | HLA-C*05:01; HLA-C*17:01 |
| 28_neoA | 78 | ORF1Ab | LLWLIINLV | HLA-A*02:01 |
| | | (nsp3) | | |
| 29_neoA | 79 | N protein | LLLPQGTTV | HLA-A*02:01; HLA-A*02:06; HLA-A*32:01; |
| | | | | HLA-B*13:02; HLA-B*15:25; HLA-B*46:01; |
| | | | | HLA-B*51:01; HLA-B*52:01; HLA-C*01:02; |
| | | | | HLA-C*02:02; HLA-C*02:09; HLA-C*03:02; |
| | | | | HLA-C*03:03; HLA-C*03:04; HLA-C*04:01; |
| | | | | HLA-C*05:01; HLA-C*07:04; HLA-C*08:01; |
| | | | | HLA-C*08:02; HLA-C*12:02; HLA-C*12:03; |
| | | | | HLA-C*14:02; HLA-C*15:02; HLA-C*16:01; |
| | | | | HLA-C*17:01; HLA-E*01:01 |
| 31_neoA | 80 | ORF1Ab | MLISAGFSV | HLA-A*02:01; HLA-A*02:06; HLA-A*32:01; |
| | | (nsp14) | | HLA-A*68:02; HLA-B*13:01; HLA-B*13:02; |
| | | | | HLA-B*48:01; HLA-B*52:01; HLA-C*01:02; |
| | | | | HLA-C*02:02; HLA-C*02:09; HLA-C*03:02; |
| | | | | HLA-C*03:03; HLA-C*03:04; HLA-C*05:01; |
| | | | | HLA-C*07:04; HLA-C*08:01; HLA-C*08:02; |
| | | | | HLA-C*12:02; HLA-C*12:03; HLA-C*15:02; |
| | | | | HLA-C*16:01; HLA-C*17:01 |
| 34_neoA | 81 | Spike | NLNESLIDV | HLA-A*02:01; HLA-A*02:06; HLA-C*05:01; |
| | | glycoprotein | | HLA-C*17:01 |
| 35_neoA | 82 | Spike | PLRVVVLSV | HLA-A*02:01 |
| | | glycoprotein | | |
| 37_neoA | 83 | ORF1ab | RLMTWLDMV | HLA-A*02:01; HLA-A*02:06; HLA-A*32:01; |
| | | (nsp6) | | HLA-C*17:01 |
| 38_neoA | 84 | Spike | RLNEVAKNV | HLA-A*02:01; HLA-A*02:06; HLA-A*32:01; |
| | | glycoprotein | | HLA-B*13:01; HLA-B*13:02; HLA-C*02:02; |
| | | | | HLA-C*02:09; HLA-C*05:01; HLA-C*15:02; |
| | | | | HLA-C*17:01 |
| 39_neoA | 85 | N protein | RLNQLESKV | HLA-A*02:01; HLA-A*32:01; HLA-C*17:01 |
| 40_neoA | 86 | Spike | SLIAYTMSV | HLA-A*02:01; HLA-A*02:06; HLA-A*32:01; |
| | | glycoprotein | | HLA-A*68:02; HLA-B*08:01; HLA-B*13:01; |
| | | | | HLA-B*13:02; HLA-B*14:02; HLA-B*46:01; |
| | | | | HLA-B*48:01; HLA-B*52:01; HLA-C*01:02; |
| | | | | HLA-C*02:02; HLA-C*02:09; HLA-C*03:02; |
| | | | | HLA-C*03:03; HLA-C*03:04; HLA-C*04:01; |
| | | | | HLA-C*05:01; HLA-C*06:02; HLA-C*07:01; |
| | | | | HLA-C*07:02; HLA-C*07:04; HLA-C*08:01; |
| | | | | HLA-C*08:02; HLA-C*12:02; HLA-C*12:03; |
| | | | | HLA-C*14:02; HLA-C*15:02; HLA-C*16:01; |
| | | | | HLA-C*17:01; HLA-E*01:01 |
| 42_neoA | 87 | ORF1ab | SLWALIISV | HLA-A*02:01; HLA-A*02:06; HLA-A*32:01; |
| | | (nsp6) | | HLA-B*13:02; HLA-B*52:01; HLA-C*08:01; |
| | | | | HLA-C*17:01 |
| 43_neoA | 88 | M protein | TLACFVLAV | HLA-A*02:01; HLA-A*02:06 |
| 45_neoA | 89 | Spike | VLNDILSRV | HLA-A*02:01; HLA-A*02:06; HLA-A*32:01; |
| | | glycoprotein | | HLA-A*68:02; HLA-B*13:02; HLA-B*46:01; |
| | | | | HLA-B*52:01; HLA-C*01:02; HLA-C*02:02; |
| | | | | HLA-C*02:09; HLA-C*04:01; HLA-C*05:01; |
| | | | | HLA-C*06:02; HLA-C*07:04; HLA-C*08:01; |
| | | | | HLA-C*08:02; HLA-C*12:02; HLA-C*12:03; |
| | | | | HLA-C*15:02; HLA-C*17:01; HLA-E*01:01 |
| 46_neoA | 90 | ORF1Ab | VLMAPISAV | HLA-A*02:01; HLA-A*02:06; HLA-A*32:01; |
| | | (nsp3) | | HLA-A*68:02; HLA-B*08:01; HLA-B*13:02; |
| | | | | HLA-B*14:02; HLA-B*15:25; HLA-B*46:01; |
| | | | | HLA-B*48:01; HLA-B*52:01; HLA-B*55:01; |
| | | | | HLA-C*01:02; HLA-C*02:02; HLA-C*02:09; |
| | | | | HLA-C*03:02; HLA-C*03:03; HLA-C*03:04; |
| | | | | HLA-C*04:01; HLA-C*05:01; HLA-C*07:04; |
| | | | | HLA-C*08:01; HLA-C*08:02; HLA-C*12:02; |
| | | | | HLA-C*12:03; HLA-C*14:02; HLA-C*15:02; |
| | | | | HLA-C*16:01; HLA-C*17:01; HLA-E*01:01 |
| 47_neoA | 91 | Spike | VLPTKLNDV | HLA-A*02:01; HLA-A*02:06; HLA-E*01:01 |
| | | glycoprotein | | |
| 48_neoA | 92 | Spike | VLFLHVTYV | HLA-A*02:01; HLA-A*02:06; HLA-A*32:01; |
| | | glycoprotein | | HLA-A*68:02; HLA-B*13:02; HLA-B*52:01; |
| | | | | HLA-C*01:02; HLA-C*02:02; HLA-C*02:09; |
| | | | | HLA-C*04:01; HLA-C*05:01; HLA-C*06:02; |
| | | | | HLA-C*07:01; HLA-C*07:02; HLA-C*07:04; |
| | | | | HLA-C*08:01; HLA-C*12:02; HLA-C*12:03; |
| | | | | HLA-C*15:02; HLA-C*16:01; HLA-C*17:01; |
| | | | | HLA-E*01:01 |
| 49_neoA | 93 | ORF1ab | WLMWLIINV | HLA-A*02:01; HLA-A*02:06 |
| | | (nsp3) | | |
| 50_neoA | 94 | ORF1ab | YLDAYNMMV | HLA-A*01:01; HLA-A*02:01; HLA-A*02:06; |
| | | (nsp14) | | HLA-A*68:02; HLA-B*13:02; HLA-B*39:01; |
| | | | | HLA-B*48:01; HLA-C*01:02; HLA-C*02:02; |
| | | | | HLA-C*02:09; HLA-C*03:03; HLA-C*03:04; |
| | | | | HLA-C*04:01; HLA-C*05:01; HLA-C*06:02; |
| | | | | HLA-C*07:01; HLA-C*07:02; HLA-C*07:04; |
| | | | | HLA-C*08:01; HLA-C*08:02; HLA-C*12:02; |
| | | | | HLA-C*14:02; HLA-C*15:02; HLA-C*16:01; |
| | | | | HLA-C*17:01; HLA-E*01:01; HLA-G*01:02; |
| | | | | HLA-G*01:03; HLA-G*01:04; HLA-G*01:06 |
| 51_neoA | 95 | ORF1ab | YLNSTNVTV | HLA-A*02:01; HLA-A*02:06; HLA-A*32:01; |
| | | (nsp3) | | HLA-A*68:02; HLA-B*13:01; HLA-B*13:02; |
| | | | | HLA-B*15:25; HLA-B*39:01; HLA-B*46:01; |
| | | | | HLA-B*48:01; HLA-B*52:01; HLA-B*56:01; |
| | | | | HLA-C*01:02; HLA-C*02:02; HLA-C*02:09; |
| | | | | HLA-C*03:02; HLA-C*03:03; HLA-C*03:04; |
| | | | | HLA-C*04:01; HLA-C*05:01; HLA-C*06:02; |
| | | | | HLA-C*07:01; HLA-C*07:02; HLA-C*07:04; |
| | | | | HLA-C*08:01; HLA-C*08:02; HLA-C*12:02; |
| | | | | HLA-C*12:03; HLA-C*14:02; HLA-C*15:02; |
| | | | | HLA-C*16:01; HLA-C*17:01; HLA-E*01:01; |
| | | | | HLA-G*01:02; HLA-G*01:03; HLA-G*01:04; |
| | | | | HLA-G*01:06 |
| 53_neoA | 96 | Spike | YLQPRTFLV | HLA-A*02:01; HLA-A*02:06; HLA-A*29:02; |
| | | glycoprotein | | HLA-A*32:01; HLA-A*68:02; HLA-B*08:01; |
| | | | | HLA-B*14:02; HLA-B*46:01; HLA-B*52:01; |
| | | | | HLA-C*01:02; HLA-C*02:02; HLA-C*02:09; |
| | | | | HLA-C*03:03; HLA-C*03:04; HLA-C*04:01; |
| | | | | HLA-C*05:01; HLA-C*06:02; HLA-C*07:01; |
| | | | | HLA-C*07:02; HLA-C*07:04; HLA-C*08:01; |
| | | | | HLA-C*08:02; HLA-C*12:02; HLA-C*12:03; |
| | | | | HLA-C*14:02; HLA-C*15:02; HLA-C*16:01; |
| | | | | HLA-C*17:01; HLA-E*01:01; HLA-G*01:02; |
| | | | | HLA-G*01:03; HLA-G*01:04; HLA-G*01:06 |
| 54_neoA | 97 | Protein 3a | YLYALVYFV | HLA-A*02:01; HLA-A*02:06; HLA-A*32:01; |
| | | | | HLA-A*68:02; HLA-C*04:01; HLA-C*05:01; |
| | | | | HLA-C*07:04; HLA-C*08:01; HLA-C*17:01 |
| 55_neoA | 98 | ORF1ab | YLMADLVYV | HLA-A*02:01; HLA-A*02:06; HLA-A*32:01; |
| | | (nsp12) | | HLA-A*68:02; HLA-B*13:01; HLA-B*13:02; |
| | | | | HLA-B*46:01; HLA-B*48:01; HLA-B*52:01; |
| | | | | HLA-C*01:02; HLA-C*02:02; HLA-C*02:09; |
| | | | | HLA-C*03:02; HLA-C*03:03; HLA-C*03:04; |
| | | | | HLA-C*04:01; HLA-C*05:01; HLA-C*06:02; |
| | | | | HLA-C*07:01; HLA-C*07:02; HLA-C*07:04; |
| | | | | HLA-C*08:01; HLA-C*08:02; HLA-C*12:02; |
| | | | | HLA-C*12:03; HLA-C*14:02; HLA-C*15:02; |
| | | | | HLA-C*16:01; HLA-C*17:01; HLA-E*01:01; |
| | | | | HLA-G*01:02; HLA-G*01:03; HLA-G*01:04; |
| | | | | HLA-G*01:06 |

TABLE 3
neo-epitope group B; CTL neoepitope

| | SEQ | | | |
|---|---|---|---|---|
| PepID | ID NO | Target protein | Sequence | HLA binding |
| 1_neoB | 99 | Spike | ASNTLVKQL | HLA-C*15:02; HLA-B*57:01; HLA-C*16:01; |
| | | glycoprotein | | HLA-B*58:01; HLA-B*58:02; HLA-C*02:02; |
| | | | | HLA-C*02:09; HLA-C*12:03; HLA-E*01:01 |
| 2_neoB | 100 | N protein | AVNTPKDHE | HLA-A*30:01 |
| 3_neoB | 101 | Protein 3a | AVSKGVHFI | HLA-C*15:02; HLA-C*17:01; HLA-A*68:02; |
| | | | | HLA-E*01:01; HLA-A*32:01; HLA-A*02:06; |
| | | | | HLA-C*02:02; HLA-C*02:09; HLA-G*01:02; |
| | | | | HLA-G*01:03; HLA-G*01:04; HLA-G*01:06; |
| | | | | HLA-C*05:01; HLA-B*13:01; HLA-C*01:02; |
| | | | | HLA-C*07:04; HLA-C*08:02; HLA-C*16:01; |
| | | | | HLA-A*30:01; HLA-C*12:03; HLA-A*02:01; |
| | | | | HLA-B*13:02; HLA-B*58:01 |
| 4_neoB | 102 | ORF1ab | AGYTPHTVV | HLA-B*52:01; HLA-C*16:01; HLA-B*13:02; |
| | | ((nsp12) | | HLA-C*12:03; HLA-C*15:02; HLA-C*01:02; |
| | | | | HLA-C*03:03; HLA-C*03:04; HLA-B*55:01; |
| | | | | HLA-C*08:01; HLA-B*51:01; HLA-C*08:02; |
| | | | | HLA-A*30:01; HLA-C*07:04; HLA-C*17:01; |
| | | | | HLA-C*03:02; HLA-B*48:01; HLA-C*12:02 |
| 5_neoB | 103 | ORF1ab | CVEASFNYL | HLA-C*17:01; HLA-C*05:01 |
| | | (nsp3) | | |
| 6_neoB | 104 | Spike | FMAGLIAIV | HLA-A*02:01; HLA-A*02:06; HLA-C*17:01; |
| | | glycoprotein | | HLA-C*08:01; HLA-C*12:02; HLA-C*02:02; |
| | | | | HLA-C*02:09; HLA-C*05:01; HLA-C*08:02; |
| | | | | HLA-C*01:02; HLA-C*12:03; HLA-C*03:03; |
| | | | | HLA-C*03:04; HLA-A*68:02; HLA-B*46:01 |
| 7_neoB | 105 | ORF1Ab | FTAHIQWMV | HLA-A*68:02; HLA-A*02:06; HLA-C*17:01; |
| | | (nsp4) | | HLA-A*02:01; HLA-C*15:02; HLA-C*02:02; |
| | | | | HLA-C*02:09; HLA-C*05:01; HLA-C*08:01; |
| | | | | HLA-A*26:01; HLA-A*25:01; HLA-C*08:02 |
| 8_neoB | 106 | ORF1ab (nsp4) | FTLNKEMYL | HLA-C*15:02; HLA-C*17:01; HLA-C*02:02; |
| | | | | HLA-C*02:09; HLA-C*01:02; HLA-C*08:01; |
| | | | | HLA-C*16:01; HLA-C*12:03; HLA-C*03:03; |
| | | | | HLA-C*03:04; HLA-A*02:06; HLA-C*12:02; |
| | | | | HLA-E*01:01; HLA-C*07:04; HLA-C*03:02; |
| | | | | HLA-C*05:01; HLA-A*68:02; HLA-C*08:02; |
| | | | | HLA-C*07:01; HLA-C*06:02; HLA-B*46:01; |
| | | | | HLA-C*07:02; HLA-A*02:01; HLA-G*01:02; |
| | | | | HLA-G*01:03; HLA-G*01:04; HLA-G*01:06; |
| | | | | HLA-C*14:02; HLA-C*04:01; HLA-B*08:01; |
| | | | | HLA-B*58:02; HLA-A*32:01 |
| 9_neoB | 107 | ORF1ab | FITENLLLF | HLA-C*02:02; HLA-C*02:09; HLA-B*46:01; |
| | | (nsp3) | | HLA-C*03:02; HLA-C*12:02; HLA-A*26:01; |
| | | | | HLA-A*25:01; HLA-C*05:01; HLA-C*08:01; |
| | | | | HLA-C*17:01; HLA-C*03:03; HLA-C*03:04; |
| | | | | HLA-C*08:02; HLA-C*12:03; HLA-A*29:02; |
| | | | | HLA-C*01:02; HLA-C*04:01; HLA-A*23:01; |
| | | | | HLA-C*16:01; HLA-B*35:01; HLA-B*53:01; |
| | | | | HLA-A*02:06; HLA-A*24:02; HLA-C*14:02; |
| | | | | HLA-B*15:02; HLA-G*01:02; HLA-G*01:03; |
| | | | | HLA-G*01:04; HLA-G*01:06; HLA-C*07:02; |
| | | | | HLA-B*15:25; HLA-A*32:01; HLA-C*15:02; |
| | | | | HLA-E*01:01 |
| 10_neoB | 108 | Spike | FSFCNDPFL | HLA-C*08:01; HLA-C*03:03; HLA-C*03:04; |
| | | glycoprotein | | HLA-C*17:01; HLA-C*01:02; HLA-C*08:02; |
| | | | | HLA-C*03:02; HLA-C*15:02; HLA-C*02:02; |
| | | | | HLA-C*02:09; HLA-A*02:06; HLA-C*05:01; |
| | | | | HLA-C*12:02; HLA-B*46:01; HLA-C*12:03 |
| 11_neoB | 109 | membrane | FLLAAVYRF | HLA-A*32:01; HLA-A*23:01; HLA-A*29:02; |
| | | glycoP | | HLA-A*02:06; HLA-A*02:01; HLA-A*24:02; |
| | | | | HLA-B*15:25; HLA-B*13:01; HLA-C*02:02; |
| | | | | HLA-C*02:09; HLA-C*08:01; HLA-B*46:01; |
| | | | | HLA-C*17:01; HLA-C*04:01; HLA-G*01:02; |
| | | | | HLA-G*01:03; HLA-G*01:04; HLA-G*01:06; |
| | | | | HLA-B*15:02; HLA-C*03:02; HLA-B*15:01; |
| | | | | HLA-C*12:02; HLA-C*05:01; HLA-C*08:02; |
| | | | | HLA-C*07:02; HLA-E*01:01; HLA-C*16:01 |
| 12_neoB | 110 | Spike | GVQGNFKNL | HLA-E*01:01; HLA-G*01:02; HLA-G*01:03; |
| | | glycoprotein | | HLA-G*01:04; HLA-G*01:06 |
| 13_neoB | 111 | ORF1 | GFFKDCSKG | HLA-A*23:01 |
| | | (nsp14) | | |
| 14_neoB | 112 | M protein | GLMWLSYFT | HLA-A*02:01 |
| 15_neoB | 113 | N protein | GTSRIGMEV | HLA-A*02:06; HLA-A*68:02; HLA-C*02:02; |
| | | | | HLA-C*02:09; HLA-C*15:02; HLA-C*17:01 |
| 16_neoB | 114 | Spike | GFQPYRVVVL | HLA-A*24:02; HLA-A*23:01 |
| | | glycoprotein | | |
| 17_neoB | 115 | ORF1ab (nsp3) | IPLLDQALV | HLA-B*07:02; HLA-B*35:01; HLA-B*35:03; |
| | | | | HLA-B*51:01; HLA-B*53:01; HLA-B*55:01; |
| | | | | HLA-B*56:01 |
| 18_neoB | 116 | N protein | ILLNKHIDG | HLA-B*08:01 |
| 19_neoB | 117 | Spike | KHADYNYKL | HLA-A*23:01; HLA-A*24:02; HLA-B*13:01; |
| | | glycoprotein | | HLA-B*13:02; HLA-B*14:02; HLA-B*37:01; |
| | | | | HLA-B*38:01; HLA-B*39:01; HLA-B*48:01; |
| | | | | HLA-B*52:01; HLA-C*04:01; HLA-C*06:02; |
| | | | | HLA-C*07:01; HLA-C*07:02; HLA-C*07:04; |
| | | | | HLA-C*08:01; HLA-C*14:02; HLA-E*01:01; |
| | | | | HLA-G*01:02; HLA-G*01:03; HLA-G*01:04; |
| | | | | HLA-G*01:06 |
| 20_neoB | 118 | ORF1ab | KIKDCVMYV | HLA-A*02:01; HLA-A*02:06; HLA-A*30:01; |
| | | (nsp6) | | HLA-A*32:01; HLA-C*02:02; HLA-C*02:09; |
| | | | | HLA-C*15:02; HLA-C*17:01 |
| 21_neoB | 119 | Spike | KAPDDFTGCV | HLA-A*02:01; HLA-B*07:02 |
| | | glycoprotein | | |
| 22_neoB | 120 | ORFA1b | KLSYGIATE | HLA-A*74:01 |
| | | (nsp13) | | |
| 23_neoB | 121 | N protein | LCLLLLDRI | HLA-B*51:01 |
| 24_neoB | 122 | Spike | LATGRLQSI | HLA-B*08:01; HLA-B*51:01; HLA-B*52:01; |
| | | glycoprotein | | HLA-C*01:02; HLA-C*03:02; HLA-C*03:03; |
| | | | | HLA-C*03:04; HLA-C*08:01; HLA-C*12:02; |
| | | | | HLA-C*12:03; HLA-C*15:02; HLA-C*16:01 |
| 25_neoB | 123 | N protein | LCLDRLNQR | HLA-A*33:03; HLA-A*31:01 |
| 26_neoB | 124 | ORF1ab | LPLTILTSK | HLA-B*55:01; HLA-B*56:01 |
| | | (nsp6) | | |
| 27_neoB | 125 | ORF1Ab | LVSAGIFGV | HLA-A*02:01; HLA-A*02:06; HLA-A*68:02; |
| | | (nsp3) | | HLA-C*02:02; HLA-C*02:09; HLA-C*05:01; |
| | | | | HLA-C*15:02; HLA-C*17:01 |
| 28_neoB | 126 | ORF1Ab | LAWLIINLV | HLA-B*51:01; HLA-B*52:01 |
| | | (nsp3) | | |
| 29_neoB | 127 | N protein | LGLPQGTTE | HLA-B*35:01 |
| 30_neoB | 128 | ORF1Ab | MPAKALRKV | HLA-B*07:02; HLA-B*35:01; HLA-B*35:03; |
| | | (nsp3) | | HLA-B*51:01; HLA-B*53:01; HLA-B*55:01; |
| | | | | HLA-B*56:01 |
| 31_neoB | 129 | ORF1Ab | MAISAGFSV | HLA-A*02:06; HLA-A*25:01; HLA-A*26:01; |
| | | (nsp14) | | HLA-A*32:01; HLA-A*68:02; HLA-B*13:01; |
| | | | | HLA-B*13:02; HLA-B*14:02; HLA-B*15:02; |
| | | | | HLA-B*35:01; HLA-B*35:03; HLA-B*46:01; |
| | | | | HLA-B*51:01; HLA-B*52:01; HLA-B*53:01; |
| | | | | HLA-B*55:01; HLA-B*56:01; HLA-B*58:01; |
| | | | | HLA-C*01:02; HLA-C*02:02; HLA-C*02:09; |
| | | | | HLA-C*03:02; HLA-C*03:03; HLA-C*03:04; |
| | | | | HLA-C*05:01; HLA-C*08:01; HLA-C*08:02; |
| | | | | HLA-C*12:02; HLA-C*12:03; HLA-C*14:02; |
| | | | | HLA-C*15:02; HLA-C*16:01; HLA-C*17:01 |
| 32_neoB | 130 | ORF1ab | NAIDSYFVL | HLA-A*02:06; HLA-A*25:01; HLA-A*26:01; |
| | | (nsp12) | | HLA-A*68:02; HLA-B*08:01; HLA-B*13:01; |
| | | | | HLA-B*13:02; HLA-B*14:02; HLA-B*15:02; |
| | | | | HLA-B*35:01; HLA-B*35:03; HLA-B*37:01; |
| | | | | HLA-B*38:01; HLA-B*39:01; HLA-B*46:01; |
| | | | | HLA-B*48:01; HLA-B*51:01; HLA-B*52:01; |
| | | | | HLA-B*53:01; HLA-B*55:01; HLA-B*56:01; |
| | | | | HLA-B*58:02; HLA-C*01:02; HLA-C*02:02; |
| | | | | HLA-C*02:09; HLA-C*03:02; HLA-C*03:03; |
| | | | | HLA-C*03:04; HLA-C*04:01; HLA-C*05:01; |
| | | | | HLA-C*06:02; HLA-C*07:01; HLA-C*07:02; |
| | | | | HLA-C*07:04; HLA-C*08:01; HLA-C*08:02; |
| | | | | HLA-C*12:02; HLA-C*12:03; HLA-C*14:02; |
| | | | | HLA-C*15:02; HLA-C*16:01; HLA-C*17:01; |
| | | | | HLA-E*01:01; HLA-G*01:02; HLA-G*01:03; |
| | | | | HLA-G*01:04; HLA-G*01:06 |
| 33_neoB | 131 | ORF1ab | NWLKDCPAI | HLA-A*24:02; HLA-A*23:01 |
| | | (nsp12) | | |
| 34_neoB | 132 | Spike | NWNESLIDD | HLA-A*24:02 |
| | | glycoprotein | | |
| 35_neoB | 133 | Spike | PLRVVVLSP | HLA-A*30:01 |
| | | glycoprotein | | |
| 36_neoB | 134 | ORF1ab | QEEMELTPI | HLA-B*37:01; HLA-B*40:01; HLA-B*40:02; |
| | | (nsp3) | | HLA-B*49:01; HLA-B*50:01 |
| 37_neoB | 135 | ORF1ab | RTMTWLDMI | HLA-A*02:06; HLA-A*32:01; HLA-B*57:01; |
| | | (nsp6) | | HLA-B*58:01; HLA-B*58:02; HLA-C*02:02; |
| | | | | HLA-C*02:09; HLA-C*15:02; HLA-C*17:01 |
| 38_neoB | 136 | Spike | RPNEVAKNT | HLA-B*07:02; HLA-B*55:01; HLA-B*56:01 |
| | | glycoprotein | | |
| 39_neoB | 137 | N protein | RLNQLESKT | HLA-A*02:01 |
| 40_neoB | 138 | Spike | SPIAYTMSV | HLA-A*25:01; HLA-A*68:02; HLA-B*07:02; |
| | | glycoprotein | | HLA-B*08:01; HLA-B*13:02; HLA-B*14:02; |
| | | | | HLA-B*35:01; HLA-B*35:03; HLA-B*39:01; |
| | | | | HLA-B*51:01; HLA-B*52:01; HLA-B*53:01; |
| | | | | HLA-B*55:01; HLA-B*56:01; HLA-C*01:02; |
| | | | | HLA-C*03:02; HLA-C*03:03; HLA-C*03:04; |
| | | | | HLA-C*07:04; HLA-C*08:01; HLA-C*08:02; |
| | | | | HLA-C*12:02; HLA-C*12:03; HLA-C*15:02; |
| | | | | HLA-C*16:01 |
| 41_neoB | 139 | ORF1ab | SAPGVFCGV | HLA-A*02:06; HLA-A*68:02; HLA-B*51:01 |
| | | (nsp4) | | |
| 42_neoB | 140 | ORF1ab | SAWALIISV | HLA-A*02:01; HLA-A*02:06; HLA-A*32:01; |
| | | (nsp6) | | HLA-A*68:02; HLA-B*13:01; HLA-B*13:02; |
| | | | | HLA-B*48:01; HLA-B*51:01; HLA-B*52:01; |
| | | | | HLA-B*55:01; HLA-B*56:01; HLA-C*01:02; |
| | | | | HLA-C*02:02; HLA-C*02:09; HLA-C*03:02; |
| | | | | HLA-C*03:03; HLA-C*03:04; HLA-C*08:01; |
| | | | | HLA-C*08:02; HLA-C*12:02; HLA-C*12:03; |
| | | | | HLA-C*15:02; HLA-C*16:01; HLA-C*17:01 |
| 43_neoB | 141 | M protein | TNACFVLAAA | HLA-A*02:01; HLA-B*56:01; HLA-A*68:02 |
| 44_neoB | 142 | ORF1ab | TAMNVLTLV | HLA-A*02:06; HLA-A*25:01; HLA-A*68:02; |
| | | (nsp6) | | HLA-B*13:02; HLA-B*46:01; HLA-B*51:01; |
| | | | | HLA-B*52:01; HLA-B*53:01; HLA-B*55:01; |
| | | | | HLA-B*56:01; HLA-C*01:02; HLA-C*02:02; |
| | | | | HLA-C*02:09; HLA-C*03:02; HLA-C*03:03; |
| | | | | HLA-C*03:04; HLA-C*05:01; HLA-C*06:02; |
| | | | | HLA-C*07:04; HLA-C*08:01; HLA-C*08:02; |
| | | | | HLA-C*12:02; HLA-C*12:03; HLA-C*15:02; |
| | | | | HLA-C*16:01; HLA-C*17:01 |
| 45_neoB | 143 | Spike | VANDILSRL | HLA-C*12:03; HLA-C*15:02; HLA-C*02:02; |
| | | glycoprotein | | HLA-C*02:09; HLA-C*03:03; HLA-C*03:04; |
| | | | | HLA-C*16:01; HLA-C*03:02; HLA-C*08:01; |
| | | | | HLA-C*12:02; HLA-C*17:01; HLA-C*01:02; |
| | | | | HLA-C*08:02; HLA-C*05:01; HLA-B*46:01; |
| | | | | HLA-E*01:01; HLA-G*01:02; HLA-G*01:03; |
| | | | | HLA-G*01:04; HLA-G*01:06; HLA-B*52:01; |
| | | | | HLA-C*06:02; HLA-B*53:01; HLA-B*58:02; |
| | | | | HLA-B*58:01; HLA-B*13:01; HLA-A*68:02; |
| | | | | HLA-B*35:03; HLA-C*07:01; HLA-C*07:04; |
| | | | | HLA-B*57:01; HLA-B*51:01; HLA-C*04:01; |
| | | | | HLA-C*07:02; HLA-C*14:02; HLA-B*56:01; |
| | | | | HLA-B*55:01; HLA-B*35:01; HLA-A*25:01; |
| | | | | HLA-B*13:02; HLA-A*32:01; HLA-B*15:02; |
| | | | | HLA-A*02:06; HLA-A*26:01; HLA-B*14:02; |
| | | | | HLA-B*48:01 |
| 46_neoB | 144 | ORF1Ab | VYMAPISAM | HLA-C*07:02; HLA-C*14:02; HLA-C*07:04; |
| | | (nsp3) | | HLA-A*24:02; HLA-G*01:02; HLA-G*01:03; |
| | | | | HLA-G*01:04; HLA-G*01:06; HLA-A*23:01; |
| | | | | HLA-B*14:02; HLA-C*07:01; HLA-C*04:01; |
| | | | | HLA-C*06:02; HLA-C*01:02; HLA-C*12:02; |
| | | | | HLA-C*03:02; HLA-B*46:01; HLA-C*16:01; |
| | | | | HLA-C*12:03; HLA-C*03:03; HLA-C*03:04; |
| | | | | HLA-E*01:01; HLA-C*08:01; HLA-C*02:02; |
| | | | | HLA-C*02:09; HLA-A*32:01; HLA-C*08:02; |
| | | | | HLA-A*29:02; HLA-B*38:01; HLA-B*15:02; |
| | | | | HLA-B*39:01; HLA-A*25:01; HLA-B*15:25; |
| | | | | HLA-A*30:02; HLA-C*17:01; HLA-B*48:01 |
| 47_neoB | 145 | Spike | VSPTKLNDG | HLA-B*57:01 |
| | | glycoprotein | | |
| 48_neoB | 146 | Spike | VSFLHVTYV | HLA-B*52:01; HLA-C*15:02; HLA-C*12:03; |
| | | glycoprotein | | HLA-C*17:01; HLA-B*51:01; HLA-C*02:02; |
| | | | | HLA-C*02:09; HLA-C*08:01; HLA-A*68:02; |
| | | | | HLA-C*12:02; HLA-C*16:01; HLA-C*01:02; |
| | | | | HLA-A*02:06; HLA-B*13:02; HLA-C*03:03; |
| | | | | HLA-C*03:04; HLA-C*06:02; HLA-E*01:01; |
| | | | | HLA-C*07:04; HLA-A*30:01; HLA-C*03:02; |
| | | | | HLA-C*07:01; HLA-C*08:02; HLA-A*32:01; |
| | | | | HLA-C*05:01; HLA-C*07:02 |
| 49_neoB | 147 | ORF1ab | WYMWLIINV | HLA-A*23:01; HLA-A*24:02 |
| | | (nsp3) | | |
| 50_neoB | 148 | ORF1ab | WCYPKCDRS | HLA-C*14:02 |
| | | (nsp14) | | |
| 51_neoB | 149 | ORF1ab | YANSTNVTI | HLA-C*03:03; HLA-C*03:04; HLA-C*08:01; |
| | | (nsp3) | | HLA-C*15:02; HLA-C*03:02; HLA-C*12:02; |
| | | | | HLA-C*12:03; HLA-C*16:01; HLA-B*52:01; |
| | | | | HLA-C*01:02; HLA-C*08:02; HLA-B*51:01; |
| | | | | HLA-C*02:02; HLA-C*02:09; HLA-C*17:01; |
| | | | | HLA-B*53:01; HLA-B*35:03; HLA-C*05:01; |
| | | | | HLA-B*46:01; HLA-B*56:01; HLA-B*55:01; |
| | | | | HLA-B*13:02; HLA-C*07:04; HLA-A*68:02; |
| | | | | HLA-B*13:01; HLA-B*35:01; HLA-B*39:01; |
| | | | | HLA-C*14:02; HLA-E*01:01; HLA-C*07:01; |
| | | | | HLA-B*58:01; HLA-C*06:02; HLA-B*58:02; |
| | | | | HLA-C*04:01; HLA-B*38:01; HLA-B*14:02; |
| | | | | HLA-B*48:01; HLA-A*32:01; HLA-B*15:02; |
| | | | | HLA-G*01:02; HLA-G*01:03; HLA-G*01:04; |
| | | | | HLA-G*01:06; HLA-C*07:02; HLA-A*02:06; |
| | | | | HLA-B*57:01; HLA-A*25:01 |
| 52_neoB | 150 | ORF1ab | YRNTLTLAV | HLA-C*06:02; HLA-C*07:01; HLA-B*39:01; |
| | | (nsp16) | | HLA-C*07:02; HLA-C*07:04; HLA-B*14:02; |
| | | | | HLA-B*38:01; HLA-B*27:05; HLA-B*27:02; |
| | | | | HLA-C*14:02; HLA-C*12:02; HLA-B*48:01; |
| | | | | HLA-C*04:01; HLA-C*02:02; HLA-C*02:09; |
| | | | | HLA-C*03:02; HLA-C*12:03 |
| 53_neoB | 151 | Spike | YNNSTNVTS | HLA-C*08:01 |
| | | glycoprotein | | |
| 54_neoB | 152 | Protein 3a | YGYALVYFA | HLA-A*02:06 |
| 55_neoB | 153 | ORF1ab | YLMADLVYT | HLA-A*02:01; HLA-A*02:06; HLA-C*17:01 |
| | | (nsp12) | | |

TABLE 4
B-cell epitopes; BCL epitope

| SEQ | | |
|---|---|---|
| ID | | |
| NO | Target protein | Sequence |
| 154 | Spike glycoprotein | NSNNLDSKVGGNY |
| | | NYLYRLFRKS |
| 155 | Spike glycoprotein | NNLDSKVGGNY |
| 156 | Spike glycoprotein | NYNYLYRLFRKS |
| 157 | Spike glycoprotein | NYNYLYRLFRKSNLK |
| | | PFERDISTEIYQA |
| 158 | Spike glycoprotein | YQAGSTPCNGVEGFN |
| 159 | Spike glycoprotein | EGFNCYFPLQSYGF |
| | | QPTNGVGYQPY |
| 160 | Spike glycoprotein | PLQSYGFQPTNGVGYQ |
| 161 | Spike glycoprotein | RVVVLSFELLHAPATV |
| | | CPGKKSTN |

TABLE 5
B-cell epitopes fused to PADRE;
BCL-HTL epitope

| SEQ ID NO | Sequence |
|---|---|
| 162 | aKXVAAWTLKAaNSNNLDSKVGG |
| | NYNYLYRLFRKS |
| 163 | aKXVAAWTLKAaNNLDSKVGGNY |
| 164 | aKXVAAWTLKAaNYNYLYRLFRKS |
| 165 | aKXVAAWTLKAaNYNYLYRLFRKSN |
| | LKPFERDISTEIYQA |
| 166 | aKXVAAWTLKAaYQAGSTPCNGVEGFN |
| 167 | aKXVAAWTLKAaEGFNCYFPLQSYGFQ |
| | PTNGVGYQPY |
| 168 | aKXVAAWTLKAaPLQSYGFQPTNGVGYQ |
| 169 | aKXVAAWTLKAaRVVVLSFELLHAPATV |
| | CPGKKSTN |

With X = L-Cyclohexylalanine, L-amino acids in single letter code are designated by upper case letters, D-amino acids are designated by lower case letters

TABLE 6

| SEQ ID | | | %identity with | %identity with |
|---|---|---|---|---|
| NO | Target protein | Sequence | SARS-CoV1 | MERS-CoV1 |
| 1 | Spike glycoprotein | ALNTLVKQL | 100 | 66 |
| 2 | N protein | ALNTPKDHI | 100 | 22 |
| 3 | ORF3 | ALSKGVHFV | 71 | 52 |
| 4 | ORF1ab | AMYTPHTVL | 100 | 44 |
| 5 | Orf1ab | CLEASFNYL | 56 | 71 |
| 6 | Spike glycoprotein | FIAGLIAIV | 100 | 57 |
| 7 | ORF1Ab (nsp4) | FLAHIQWMV | 86 | 11 |
| 8 | ORF1ab (nsp4/pplab) | FLLNKEMYL | 100 | 83 |
| 9 | ORF1ab (nsp3) | FLTENLLLY | 67 | 100 |
| 10 | Spike glycoprotein | FQFCNDPFL | 50 | 55 |
| 11 | membrane glycoP | FVLAAVYRI | 100 | 66 |
| 12 | Spike glycoprotein | GKQGNFKNL | 75 | 77 |
| 13 | ORF1(pplab) | GLFKDCSKV | 89 | 70 |
| 14 | M protein | GLMWLSYFI | 89 | 77 |
| 15 | N protein | GMSRIGMEV | 100 | 44 |
| 16 | Spike glycoprotein | GYQPYRVVVL | 100 | 20 |
| 17 | Orf1ab (ppla/nsp3) | ILLLDQALV | 100 | 66 |
| 18 | N protein | ILLNKHIDA | 100 | 66 |
| 19 | Spike glycoprotein | KIADYNYKL | 100 | 77 |
| 20 | ORF1ab (nsp6) | KLKDCVMYA | 100 | 88 |
| 21 | Spike glycoprotein | KLPDDFTGCV | 90 | 80 |
| 22 | ORFA1b (pplab) | KLSYGIATV | 100 | 77 |
| 23 | N protein | LALLLLDRL | 100 | 70 |
| 24 | Spike glycoprotein | LITGRLQSL | 100 | 66 |
| 25 | N protein | LLLDRLNQL | 100 | 70 |
| 26 | ORF1ab (nsp6) | LLLTILTSL | 78 | 55 |
| 27 | ORF1Ab (pplab/nsp3) | LLSAGIFGA | 100 | 87 |
| 28 | ORF1Ab (nsp3) | LMWLIINLV | 67 | 66 |
| 29 | N protein | LQLPQGTTL | 100 | 66 |
| 30 | ORF1Ab (nsp3) | MLAKALRKV | 67 | 77 |
| 31 | ORF1Ab | MMISAGFSL | 100 | 66 |
| 32 | ORF1ab | NLIDSYFW | 89 | 77 |
| 33 | ORF1ab | NLLKDCPAV | 89 | 66 |
| 34 | Spike glycoprotein | NLNESLIDL | 100 | 88 |
| 35 | Spike glycoprotein | PYRWVLSF | 100 | 55 |
| 36 | ORF1ab (nsp3) | QLEMELTPV | 80 | 75 |
| 37 | ORF1ab(nsp6) | RIMTWLDMV | 75 | 0 |
| 38 | Spike glycoprotein | RLNEVAKNL | 100 | 0 |
| 39 | N protein | RLNQLESKM | 89 | 71 |
| 40 | Spike glycoprotein | SIIAYTMSL | 89 | 77 |
| 41 | Orf1ab (nsp4) | SLPGVFCGV | 78 | 66 |
| 42 | Orf1ab (nsp6) | SMWALIISV | 89 | 66 |
| 43 | M protein | TLACFVLAAV | 100 | 60 |
| 44 | Orf1ab | TLMNVLTLV | 89 | 71 |
| 45 | Spike glycoprotein | VLNDILSRL | 100 | 67 |
| 46 | ORF1Ab (nsp3) | VQMAPISAM | 89 | 44 |
| 47 | Spike glycoprotein | VSPTKLNDL | 89 | 44 |
| 48 | Spike glycoprotein | VVFLHVTYV | 100 | 62 |
| 49 | Orf1ab | WLMWLIINL | 86 | 44 |
| 50 | ORF1ab | YLDAYNMMI | 100 | 57 |
| 51 | ORF1ab (nsp3) | YLNSTNVTI | 87 | 66 |
| 52 | Orf1ab | YLNTLTLAV | 100 | 88 |
| 53 | Spike glycoprotein | YLQPRTFLL | 67 | 88 |
| 54 | Orf3a | YLYALVYFL | 89 | 62 |
| 55 | Orf1ab | YTMADLVYA | 100 | 88 |

TABLE 7

| SEQ | | | | | | | |
|---|---|---|---|---|---|---|---|
| ID | | | | tetramer | Trm | ELIspot | Patient |
| NO | PepID | sequence | target_protein | response | response | (YES/NO) | response |
| 91 | 47_neoA | VLPTKLNDV | Spike glycoprotein_RBD | medium | Yes | No | Yes |
| 60 | 6_neoA | FLAGLIAIV | Spike glycoprotein | high | Yes | Yes | Yes |
| 146 | 48_neoB | VSFLHVTYV | Spike glycoprotein | high | Yes | Yes | Yes |
| 92 | 48_neoA | VLFLHVTYV | Spike glycoprotein | high | No | Yes | Yes |
| 95 | 51_neoA | YLNSTNVTV | nsp3 | high | Yes | Yes | |
| 97 | 54_neoA | YLYALVYFV | Protein 3a | high | Yes | Yes | |
| 104 | 6_neoB | FMAGLIAIV | Spike glycoprotein | medium | No | Yes | |
| 48 | 48_wt | VVFLHVTYV | Spike glycoprotein | high | No | Yes | Yes |
| 56 | l_neoA | ALNTLVKQV | Spike glycoprotein | high | Yes | Yes | Yes |
| 3 | 3_wt | ALSKGVHFV | Protein 3a | medium | No | Yes | Yes |
| 42 | 42_wt | SMWALHSV | nsp6 | high | Yes | Yes | Yes |
| 153 | 55_neoB | YLMADLVYT | nsp12 | medium | No | Yes | |
| 76 | 26_neoA | LLLTILTSV | nsp6 | medium | Yes | Yes | Yes |
| 140 | 42_neoB | SAWALIISV | nsp6 | high | Yes | Yes | Yes |
| 83 | 37_neoA | RLMTWLDMV | nsp6 | high | No | Yes | Yes |
| 135 | 37_neoB | RTMTWLDMI | nsp6 | high | No | Yes | Yes |
| 20 | 20_wt | KLKDCVMYA | nsp6 | medium | Yes | No | |
| 8 | 8_wt | FLLNKEMYL | nsp4 | medium | No | Yes | Yes |
| 139 | 41_neoB | SAPGVFCGV | nsp4 | high | No | Yes | YES |
| 77 | 27_neoA | LLSAGIFGV | nsp3 | high | No | Yes | Yes |
| 59 | 5_neoA | CLEASFNYV | nsp3 | medium | Yes | No | Yes |
| 125 | 27_neoB | LVSAGIFGV | nsp3 | high | Yes | Yes | Yes |
| 36 | 36_wt | QLEMELTPV | nsp3 | medium | Yes | No | Yes |
| 78 | 28_neoA | LLWLIINLV | nsp3 | high | No | Yes | Yes |
| 30 | 30_wt | MLAKALRKV | nsp3 | high | No | Yes | Yes |
| 52 | 52_wt | YLNTLTLAV | nsp16 | neg | No | Yes | Yes |
| 101 | 3_neoB | AVSKGVHFI | Protein 3a | medium | No | Yes | |
| 31 | 31_wt | MMISAGFSL | nsp14 | high | No | Yes | Yes |
| 105 | 7_neoB | FTAHIQWMV | nsp4 | medium | No | Yes | |
| 67 | 15_neoA | GLSRIGMEV | N protein | high | No | No | |
| 113 | 15_neoB | GTSRIGMEV | N protein | high | No | No | |
| 120 | 22_neoB | KLSYGIATE | nsp13 | high | No | No | |
| 75 | 25_neoA | LLLDRLNOV | N protein | medium | No | Yes | |
| 74 | 24_neoA | LLTGRLQSV | Spike glycoprotein | high | No | Yes | |
| 22 | 22_wt | KLSYGIATV | nsp13 | low | No | Yes | Yes |
| 32 | 32_wt | NLIDSYFW | nsp12 | medium | Yes | Yes | Yes |
| 23 | 23_wt | LALLLLDRL | N protein | medium | Yes | Yes | Yes |
| 33 | 33_wt | NLLKDCPAV | nsp12 | medium | No | Yes | |
| 34 | 34_wt | NLNESLIDL | Spike glycoprotein | medium | No | No | |
| 85 | 39_neoA | RLNQLESKV | N protein | medium | Yes | No | |
| 84 | 38_neoA | RLNEVAKNV | Spike glycoprotein | high | No | No | |
| 79 | 29_neoA | LLLPQGTTV | N protein | medium | Yes | No | Yes |
| 66 | 14_neoA | GLMWLSYFV | M protein | medium | No | Yes | Yes |
| 86 | 40_neoA | SLIAYTMSV | Spike glycoprotein | medium | No | Yes | |
| 90 | 46_neoA | VLMAPISAV | nsp3 | medium | No | Yes | |
| 49 | 49_wt | WLMWLIINL | nsp3 | high | No | No | |
| 70 | 19_neoA | KLADYNYKV | Spike glycoprotein_RBD | low | NA | Yes | Yes |

TABLE 8

| SEQ | | | | tetramer | Trm | ELIspot | Patient |
|---|---|---|---|---|---|---|---|
| ID | PepID | sequence | target_protein | response | (response) | (YES/ | response |
| 91 | 47_neoA | VLPTKLNDV | Spike glycoprotein_RBD | medium | Yes | NO) | Yes |
| 70 | 19_neoA | KLADYNYKV | Spike glycoprotein_RBD | low | NA | Yes | Yes |
| 60 | 6_neoA | FLAGLIAIV | Spike glycoprotein | high | Yes | Yes | Yes |
| 146 | 48_neoB | VSFLHVTYV | Spike glycoprotein | high | Yes | Yes | Yes |
| 92 | 48_neoA | VLFLHVTYV | Spike glycoprotein | high | No | Yes | Yes |
| 48 | 48_wt | VVFLHVTYV | Spike glycoprotein | high | No | Yes | Yes |
| 56 | l_neoA | ALNTLVKQV | Spike glycoprotein | high | Yes | | Yes |
| 3 | 3_wt | ALSKGVHFV | Protein 3a | medium | No | | Yes |
| 42 | 42_wt | SMWALIISV | nsp6 | high | Yes | Yes | Yes |
| 76 | 26_neoA | LLLTILTSV | nsp6 | medium | Yes | | Yes |
| 140 | 42_neoB | SAWALIISV | nsp6 | high | Yes | | Yes |
| 83 | 37_neoA | RLMTWLDMV | nsp6 | high | No | | Yes |
| 135 | 37_neoB | RTMTWLDMI | nsp6 | high | No | | Yes |
| 8 | 8_wt | FLLNKEMYL | nsp4 | medium | No | | Yes |
| 139 | 41_neoB | SAPGVFCGV | nsp4 | high | No | | YES |
| 77 | 27_neoA | LLSAGIFGV | nsp3 | high | No | Yes | Yes |
| 59 | 5_neoA | CLEASFNYV | nsp3 | medium | Yes | | Yes |
| 125 | 27_neoB | LVSAGIFGV | nsp3 | high | Yes | | Yes |
| 36 | 36_wt | QLEMELTPV | nsp3 | medium | Yes | | Yes |
| 78 | 28_neoA | LLWLIINLV | nsp3 | high | No | | Yes |
| 30 | 30_wt | MLAKALRKV | nsp3 | high | No | | Yes |
| 52 | 52_wt | YLNTLTLAV | nsp16 | neg | No | Yes | Yes |
| 31 | 31_wt | MMISAGFSL | nsp14 | high | No | | Yes |
| 22 | 22_wt | KLSYGIATV | nsp13 | low | No | Yes | Yes |
| 32 | 32_wt | NLIDSYFW | nsp12 | medium | Yes | | Yes |
| 23 | 23_wt | LALLLLDRL | N protein | medium | Yes | Yes | Yes |
| 79 | 29_neoA | LLLPQGTTV | N protein | medium | Yes | | Yes |
| 66 | 14_neoA | GLMWLSYFV | M protein | medium | No | Yes | Yes |

### EXAMPLES

### Example 1

The COVID-19 pandemic is caused by severe acute respiratory syndrome coronavirus-2 (SARS-CoV-2) which enters the body principally through the nasal and larynx mucosa and progress to the lungs through the respiratory tract. SARS-CoV-2 replicates efficiently in respiratory epithelial cells motivating the development of alternative and rapidly scalable vaccine inducing mucosal protective and long-lasting immunity. Here, the inventors present a multi-target CD8 T cell peptide COVID-19 vaccine design targeting several structural (S, M, N) and non-structural (NSPs) SARS-CoV-2 proteins with selected epitopes in conserved regions on the SARS-CoV-2 genome. They observe that a single subcutaneous injection of several peptides induces robust immunogenicity measured by IFNγ ELIspot. Upon tetramer characterization, they found that a series of epitopes induce a strong proportion of virus-specific CD8 T cells expressing CD103, CD44, CXCR3 and CD49a, the specific phenotype of tissue-resident memory T lymphocytes (Trm). Finally, they observe broad cellular responses, as characterized by IFNγ production, upon restimulation with structural and non-structural protein-derived epitopes using blood T cells isolated from convalescent asymptomatic, moderate and severe COVID-19 patients.

Humoral and cellular adaptive immunity are different and complementary immune defenses engaged by the body to clear viral infection. While neutralizing antibodies have the capacity to block virus binding to its entry receptor expressed on human cells, memory T lymphocytes have the capacity to eliminate infected cells and are required for viral clearance. However, viruses evolve quickly, and their antigens are prone to mutations to avoid recognition by the antibodies (phenomenon named ‘antigenic drift’). This limitation of the antibody-mediated immunity could be addressed by the T-cell mediated immunity, which is able to recognize conserved viral peptides from any viral proteins presented by virus-infected cells. Thus, by targeting several proteins and conserved regions on the genome of a virus, T-cell epitope-based vaccines are less subjected to mutations and may work effectively on different strains of the virus. We design a multi-target T cell-based vaccine containing epitope regions optimized for CD8+ T cell stimulation that would drive long-lasting cellular immunity with high specificity, avoiding undesired effects such as antibody-dependent enhancement (ADE) and antibody-induced macrophages hyperinflammation observed in the COVID-19. The present results showed that a single injection of selected CD8 T cell epitopes induces memory viral-specific T-cell responses with a phenotype of tissue-resident memory T cells (Trm). Trm has attracted a growing interest for developing vaccination strategies since they act as immune sentinels in barrier tissue such as the respiratory tract and the lung. Because of their localization in tissues, they are able to immediately recognize infected cells and, because of their memory phenotype, to rapidly respond to viral infection by orchestrating local protective immune responses to eliminate pathogens. Lastly, such multiepitope-based vaccination platform uses robust and well-validated synthetic peptide production technologies that can be rapidly manufactured in a distributed manner.

### Results

### CD8 T-Cell Epitopes Selection and Neo-Epitopes Design

Previous research in SARS-CoV-1 suggests that the structural Spike (S) protein is one of the main antigenic components responsible for inducing the host immune responses. However recent evaluation of asymptomatic, moderate and severe convalescent COVID-19 patients showed broad and robust T-cell responses not only on Spike but also on membrane (M), nucleocapsid (N) proteins and several ORFs non-structural proteins (nsp). Deep sequencing data of tens of thousands SARS-CoV-2 genomes from all over the world identified regions that have remained largely invariant to date, and others that have already accumulated significant diversity with several hundreds of point mutations (SNPs) in some key viral proteins, such as the Spike glycoprotein which also displayed a large number of recurrent mutations and several homoplasic site highlighting a possible convergent evolution and adaptation of SARS-CoV-2 to the human host. COVID-19 vaccines reliance on a single antigen (Spike) bearing recurrent mutations and homoplasic sites, as occurring with Spike monovalent vaccines under development, is not without risk of antigen drift, selection pressure and immune evasion.

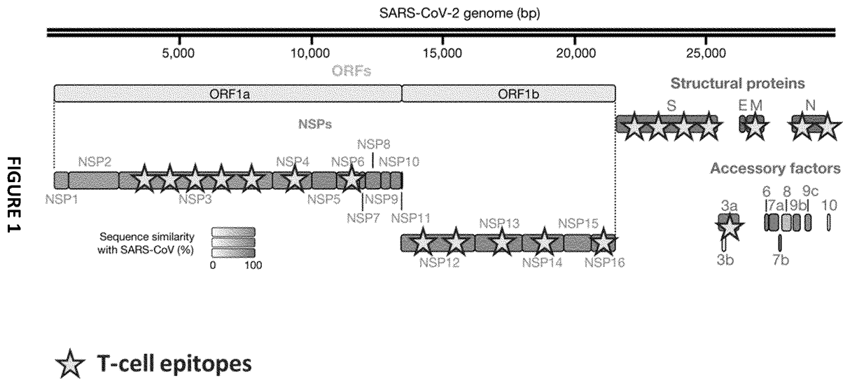

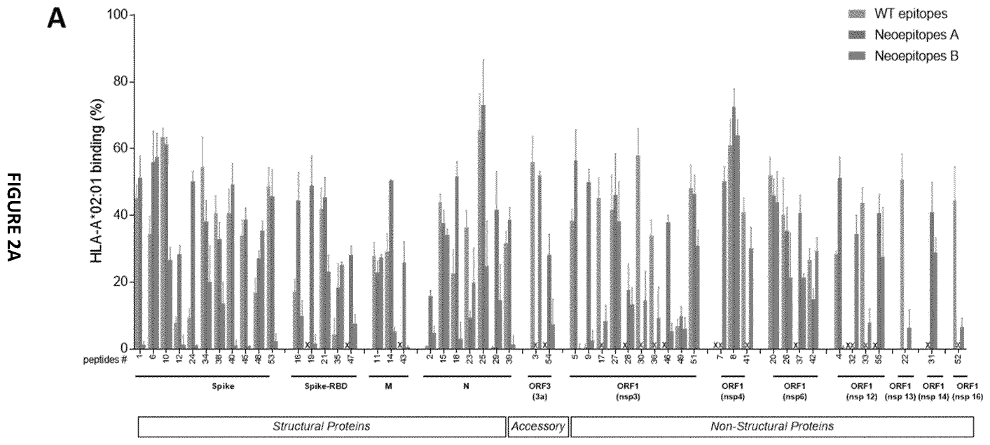

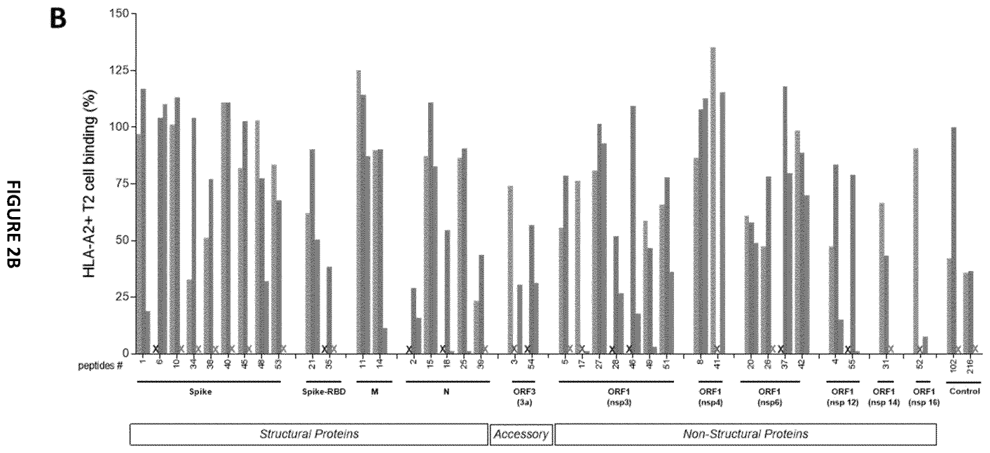

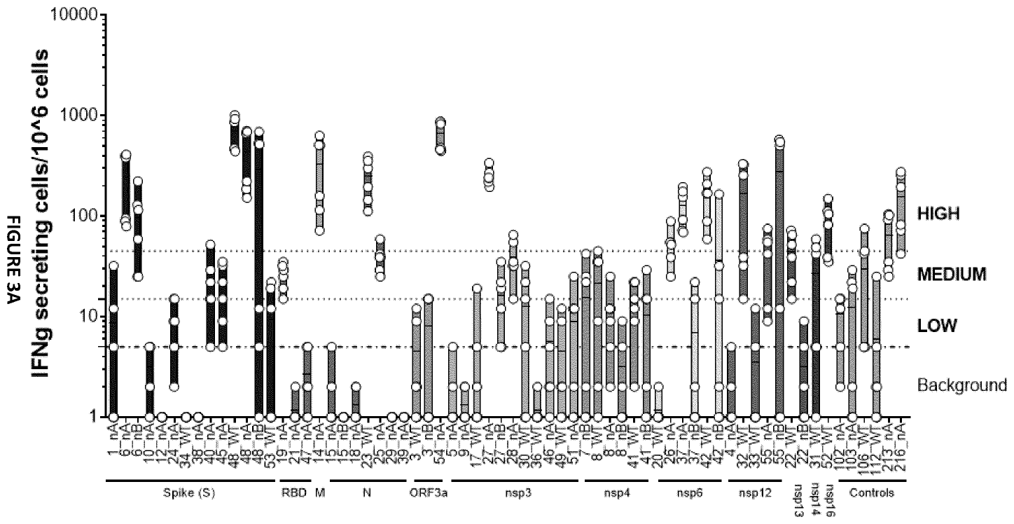

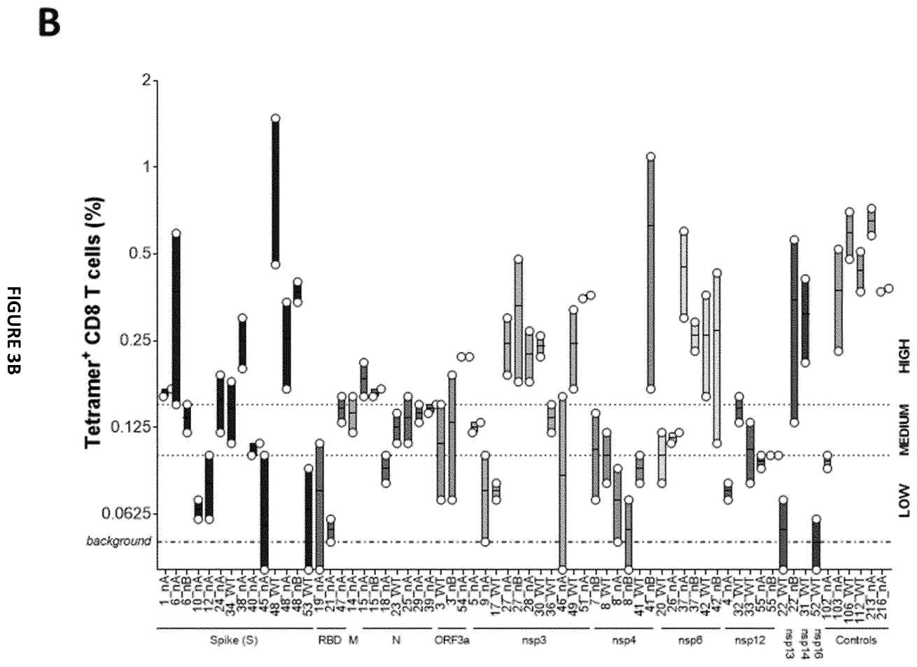

Using bioinformatics approaches of immune deconvolution to identify T cell epitopes intersected with predicted SARS-CoV-2 T-cell epitopes reported in early manuscripts, population HLA diversity, protein sequence similarity and immunogenicity observed with previous CoVs homologues and the number of copies per virion of SARS-COV-1 (estimated to approximatively 100 copies for S, 1 000 for M, 2 000 for N and only 20 for E proteins), the inventors identified and selected 55 HLA-A2-restricted T-cell epitopes (9-10 mer peptides) derived from 11 of the 29 SARS-CoV-2 proteins in human cells: 3 out of the 4 structural proteins (S, M, N), the largest accessory proteins (ORF3a) and 7 out of the 16 ORF1a/b non-structural proteins (nsp3, nsp4, nsp6, nsp12, nsp13, nsp14, nsp16) (FIG. 1 and Table 1). The inventors then designed 400 mutated peptides for each of the epitopes based on their knowledge of key fixed-anchor positions to enhance HLA binding and increase their immunogenicity potential. These 22 000 mutated peptides were first screened using in-silico bioinformatic analyses (e.g. IEDB Immune epitope database, netMHCpan EL 4.0 algorithm) and a first series of the most optimized mutant for each epitope was selected (neo-epitopes A, see Table 2). In parallel, 22 000 in silico HLA-A*0201-peptide docking models were generated using computational tools and analyzed using newly developed proprietary Artificial Intelligence (AI) algorithms based on binding energy calculations and reduction (MabSilico, France). A second series of the most optimized mutant for each epitope was selected (neo-epitopes B, see Table 3). Finally, SARS-CoV-2 genetic evolution analyses through the alignment of 23 085 sequences (https://macman123.shinyapps.io/ugi-scov2-alignment-screen/) identified recurrent mutation (SNPs) and homoplasic site in SARS-CoV-2 genomes isolated globally, particularly in the Spike protein which contain the D614G mutation and which identified the new dominant SARS-CoV-2 variant emerged in February in Europe, then spread worldwide and became the most prevalent form. The inventors eliminated T cell epitopes with recurrent mutation and homoplasic site in order to cover all circulating SARS-CoV-2 strains and anticipate future evolution of the virus in hotspot mutation regions.

CD8 T-Cell Epitopes Elicit Tissue-Resident Memory (Trm) Viral-Specific T Cells In-Vivo 134 WT and mutated peptides (neo-epitopes A and B) were produced using synthetic peptide synthesis (Proteogenix, France). HLA-A2 binding property characterization at 37° C., using UV peptide exchange assay on H LA-A*0201 monomer, showed that the majority of selected WT epitopes binds to HLA-A2 with good efficacy (FIG. 2A) as compared to our MEMOPI® internal positive neoepitope control (mutated peptide with increased HLA-A*0201 binding and in-vivo immunogenicity). HLA-A2 binding was increased with several neoepitopes A and/or B, particularly when the corresponding WT peptide showed weak (<15%: peptides 2, 9, 12, 24, 29 and 35) or intermediate (15-30%: peptides 4, 14, 16, 18 and 48) HLA-A2 stability at 37° C. Similarly, several neoepitopes A increased peptide binding to HLA-A2 expressing human cells (TAP-deficient human cell line (T2) lacking the ability to transport peptide fragments to the endoplasmic reticulum to form stable pMHCI) particularly when the corresponding WT peptide showed low (<30%: peptides 34, 39) or intermediate (30-60%: peptides 4, 5, 21, 26, 38) HLA-A2 stability at 37° C. (FIG. 2B). Altogether, bioinformatic and AI-based mutation increased peptides stability with HLA-A2 (recombinant or expressed by cells) for 52% (26/50) of evaluated CD8 T-cell epitopes.

60 peptides (at least one WT or mutated peptides for each T-cell epitopes outside homoplasic site) has been selected based on HLA-A2 binding, peptide stability and SARS-CoV-2 genome stability for further in-vivo immunogenicity evaluation in HLA-A2.1 transgenic mice. Mice received a single subcutaneous injection of each peptide combined with the universal PADRE helper T-cell epitope and emulsified in Montanide ISA-51 adjuvant. Immunogenicity was assessed in the spleen and draining lymph nodes 11 days after vaccination by ex-vivo restimulation and tetramer phenotypic characterization with the corresponding WT peptide to evaluate cross-reactivity of elicited T cell response towards WT epitopes. CD8 T cells IFNγ ELIspot restimulation showed large immunogenicity in-vivo responses elicited by single vaccination with 14 out of 60 (23%) positive CD8 T cells epitopes derived from 8 out of 11 selected proteins (FIG. 3A). Similarly, broad immunogenicity response was observed by HLA-A*0201-tetramer flow cytometry analyses to a higher number of epitopes since 44 out of 60 (73%) evaluated peptides, derived from 10/11 selected proteins, exhibited significant frequency (0.1-1%) of viral-specific CD8 T cells (FIG. 3B). Phenotypic characterization of Tetramer+ cells showed that 17 out of 44 (39%) positive peptides elicited viral-specific CD8 T cells with mainly a Trm phenotype: co-expressing the memory marker CD44, the CD103 αE integrin, the Th1-biased CXCR3 chemokine receptor and to a lesser extent the CD49a al integrin (FIG. 4). Altogether, these data showed that MEMOPI® peptide vaccination against selected SARS-CoV-2 epitopes elicits robust and broad Th1-biased immunogenicity against several structural (S, M, N) and non-structural proteins in HLA-A2 expressing mice and that several peptides induce viral-specific memory CD8 T cells displaying all characteristics of T lymphocyte sentinels in barrier tissues.

### CD8 T-Cell Epitopes Ex-Vivo Reactivity in Asymptomatic and Convalescent COVID-19 Individuals Compared to Unexposed Healthy Donors

In order to identify and select naturally SARS-CoV-2 CD8 T cell immunodominant epitopes, peripheral blood mononuclear cells (PBMC) from asymptomatic and moderate or severe COVID-19 patients with a previously confirmed (at least one month before sampling) and recovered SARS-CoV-2 infection were restimulated ex-vivo for one week with each of the isolated selected 60 peptides derived from 11 proteins. IFNγ responses over 48 hours of restimulation with HLA-A2+ Tap-deficient (T2) human cells were analyzed and compare to PBMC of unexposed healthy donors. The serology of all unexposed healthy donors was negative while all asymptomatic, moderate and severe COVID-19 individuals were IgG+ for anti-Spike antibodies. As recently reported for CD4+ T cells response, the inventors first observed some positive IFNγ response in unexposed donors to few CD8 T cells epitopes, in particular for those derived from structural proteins (Spike and N proteins but no response to the Spike_RBD or M protein) while CD8 T cell responses to non-structural proteins were limited to nsp12 and nsp13 (FIG. 5). In contrast, broad positive IFNγ response was reproducibly measured against 43 out of 60 selected CD8 T cells epitopes derived from all evaluated SARS-CoV-2 proteins (n=11) in moderate/severe COVID-19 convalescent patients as well as in asymptomatic individuals but to a lesser extent.

Altogether, the inventors identified in previously infected SARS-CoV-2 individuals 22 significantly different CD8 T cell immunodominant epitopes against 3 structural proteins (S, M, N), 1 accessory factor (ORF3a) and 7 non-structural proteins (nsp3, nsp4, nsp6, nsp12, nsp13, nsp14, nsp16) as compared to unexposed healthy donors. 18 of these epitopes are of particular interest for vaccination since able to elicit also in-vivo CD8 T cell immunogenicity against all 11 structural and non-structural SARS-CoV-2 proteins after a single peptide injection in HLA-A2 expressing mice. The inventors selected a combination of 12 CD8 T cells epitopes based on manufacturing, HLA-I coverage, previous CoVs homology and SARS-CoV-2 proteins diversity considerations (Table 9). These 12 epitopes covered the 11 selected proteins, 1 epitope/protein excepting Spike for which 2 epitopes (including 1 RBD epitope) have been selected. Bioinformatic analyses illustrate these 12 epitopes are not restricted to HLA-A*0201 allele, hence are predict (netMHC score <1) to bind efficiently to different HLA-I (A, B, C) alleles with high genetic coverage in all geographical region of the world. Despite HLA polymorphism and different worldwide HLA-I distribution, the combination of these 12 T cell epitopes should induce at least 1 to 3 positive peptides responses in all individuals globally and achieve the 60-70% ‘herd immunity’ threshold with at least 3 to 7 positive peptides responses in in each geographical region (Table 10).

### Methods

### Study Approval

Animal housing and procedures have been conducted according to the guidelines of the French Agriculture Ministry and were approved by the regional ethical committee (APAFIS 25256.) as well as according to the guidelines of Jackson Laboratory (Bar Harbor, USA) and approved by the Institutional Animal Care and Use Committee (IACUC #20031). Human studies were performed under the clinical protocol COVEPIT-1 approved by French Central Ethic Committee (CPP) and registered by the French Regulatory Authority (ANSM) under the ID-RCB no 2020-A01654-35. Written informed consent has been obtained from each of the participating subject.

### HLA-A*0201-Peptide Binding Evaluation

T-cell WT and mutated peptides binding property on HLA-A2 has been evaluated using the Flex-T HLA-A*02:01 monomer ultraviolet (UV) exchange assay according to the manufacturer recommendation (Biolegend, San Diego, USA). HLA-A*02:01 monomer (200 μg/ml) were exposed to a 366-nm UV lamp in the presence or absence of 400 μM of peptide. After UV-exposure, HLA-peptide complexes were incubated at 37° C. for 30 min to promote unfolding of peptide-free HLA molecule. HLA-peptide complexes stability was detected by ELISA with 132-microglobulin coated antibodies and incubation of 3 ng/ml of complexes for 1 h at room temperature under shaking condition. Avidin-HRP were used to reveal stable biotinylated HLA-peptide complexes and absorbance was monitored at 450 nm. Data are expressed as percentage of binding relative to an MEMOPI® internal positive control neoepitope. MEMOPI® internal positive control neoepitope is a mixture of MPS-216 (SEQ ID NO: 171) and MPS-102 (SEQ ID NO: 172).

### SARS-CoV-2 Genome Diversity Evaluation

A visualization tool was used to determine T-cell and B-cell epitope location in SARS-CoV-2 genomes according to single nucleotide polymorphism (SNPs) and homoplasic site (https://macman123.shinyapps.io/ugi-scov2-alignment-screen/). 23,085 SARS-CoV-2 genomes isolated from patients worldwide were aligned against the Wuhan-Hu-1 reference genome NC_045512.2. A total of 8,667 SNPs has been identified corresponding to 308 homoplasic sites with recurrent mutations. Peptides have been blasted with tblastn algorithm against the Wuhan-Hu-1 reference genome NC_045512.2 to determine the nucleotide coordinates for each peptide. The online tool was then used to identify the peptides corresponding to a homoplasic site.

### T-Cell Epitopes Immunization

B6.Cg-Immp2ITg(HLA-A/H2-D)2Enge/J (HLA-A2.1) transgenic mice (The Jackson Laboratory, Bar Harbor, USA) received a single subcutaneous injection of 6 SARS-CoV-2 peptides (50 μg each, WT and mutated peptide of a same epitope have not been evaluated in same mice) plus the universal PADRE helper T-cell epitope emulsified in Montanide ISA-51 adjuvant. Immunization was measured 11 days after injection. 3 males and 3 females have been evaluated per group. Freshly harvested spleen and draining lymph nodes have been pooled by sex per group and analyzed by flow cytometry analyses. CD8+ T cells have been isolated using MACS microbeads and restimulated individually with each evaluated peptide. The frequency of IFNγ-secreting CD8+ T cells was measured by ELIspot in parallel of tetramer staining for each peptide evaluated by flow cytometry. Control Memopi® peptides are a mixture of MPS-216 (SEQ ID NO: 171), MPS-102 (SEQ ID NO: 172), MPS-112 (SEQ ID NO: 173), MPS-106 (SEQ ID NO: 174), MPS-213 (SEQ ID NO: 175), and MPS-103 (SEQ ID NO: 176) plus the universal PADRE helper T-cell epitope emulsified in Montanide ISA-51 adjuvant.

### COVID-19 Patients Cohorts

All subjects were enrolled in the COVEPIT-1 clinical trial, an open-label, multicentric and prospective study with minimal risk and constraints designed to assess the memory T cell in subjects who recovered from COVID-19. Subjects were enrolled at the Groupement Santé du Bataillon des Marins-Pompiers (Marseille-France) and GHR MSA—Hôpital Emile Muller (Mulhouse, France). The main objective was to test the subject' memory T cells reactivity to a selection of SARS-CoV-2 antigens. Enrolled subjects must have a proven COVID-19 infection which recovered 1 to 6 months before study entry. Eligible subjects are male and female of 18 to 70 years old diagnosed for COVID-19 using a PCR test from a nasal and/or oropharyngeal swab, and/or a serological test, and/or a chest CT with lesions suggestive of COVID-19. Subjects were excluded if pregnant or breastfeeding, unable to fulfill the protocol requirement, with an history of cancer 5 years prior to study entry (except for localized or in situ cancer), history of head injury or sepsis 1 year prior to study entry, chronic infections (e.g. HIV infection, chronic hepatitis B, active viral hepatitis C or bacterial or fungal infection) requiring a systemic treatment in the month prior to COVID-19, disease (auto immune or inflammatory disease, transplant recipients . . . ) requiring a immunosuppressive or immunomodulator treatment and/or, corticosteroids at an equivalent dose of prednisone >10 mg/d for more than 15 days or >40 mg/d for the last 15 days prior to COVID-19; corticosteroid during COVID-19 were not considered as an exclusion criterion.

Ex-Vivo PBMC Restimulation with SARS-CoV-2 Peptide

PBMC were isolated after a Ficoll density-gradient centrifugation and a red blood cell lysis. HLA-A2 phenotyping was performed by flow cytometry (clone BB7.2, BD Bioscience). Ex-vivo stimulation protocol was adapted from a previously described protocol (Mitra, A. et al. Nature Communications 11, 1839 (2020)). HLA-A2+ positive PBMC (106/well) were incubated in RPMI 1640 containing 10 mM HEPES, 2 mM L-glutamine, 1 mM Sodium Pyruvate, 2% human AB serum, 10% bovine serum and non-essential amino acids in 48-well plates. During the first week of culture, PBMC were cultured with 3 μg/mL of each isolated peptides and IL-21 (30 ng/mL; Miltenyi, Paris France). Fresh medium containing IL-21 (30 ng/mL), IL-7 (5 ng/mL; BioRad, Paris France), and IL-2 (10 ng/mL; Miltenyi, Paris France) and peptide-loaded HLA-A2+ Tap-deficient (T2) cells were added to the culture for the next two days. Ex vivo T-cell viral stimulation was evaluated by IFNΥ supernatant quantification (BD Biosciences, US). The percentage of background IFNΥ secretion was determined by the response of PBMC co-cultured with non-loaded T2 cells and negative control peptide, then fold change was calculated over the IFNΥ secretion background for each donor.

### Statistical Analysis

Continuous variables were expressed as the mean±SEM, unless otherwise indicated, and raw data were compared with nonparametric tests: Mann-Whitney for 2 groups or Kruskall-Wallis with Dunn's comparison when the number of groups was >2. P values of <0.05 were considered statistically significant. All statistical analyses were performed on GraphPad Software (GraphPad Software, San Diego, Calif.).

### Example 2: CoVepiT

CoVepit is the combination of 13 following peptides

aKXVAAWTLKAAa, a Pan DR HTL epitope (SEQ ID NO: 170)

12 CTL epitope HLA restricted epitopes:
KLADYNYKV versus Spike glycoprotein RBD (SEQ ID NO: 70), neoepitope of 19-wt (SEQ ID NO: 19)
VSFLHVTYV versus Spike glycoprotein (SEQ ID NO: 146), neoepitope of 48-wt (SEQ ID NO: 48)
LALLLLDRL versus N protein (SEQ ID NO: 23)
GLMWLSYFV versus M protein (SEQ ID NO: 66), neoepitope of 14-wt (SEQ ID NO: 14)
YLYALVYFV versus Protein 3a (SEQ ID NO: 97), neoepitope of 54-wt (SEQ ID NO: 54)
LLSAGIFGV versus nsp3 (SEQ ID NO: 77), neoepitope of 27-wt (SEQ ID NO: 27)
FLLNKEMYL versus nsp4 (SEQ ID NO: 8)
SMWALIISV versus nsp6 (SEQ ID NO: 42)
NLIDSYFVV versus nsp12 (SEQ ID NO: 32)
KLSYGIATV versus nsp13 (SEQ ID NO: 22)
MMISAGFSL versus nsp14 (SEQ ID NO: 31)
YLNTLTLAV versus nsp16 (SEQ ID NO: 52).
The peptides are mixed and emulsified in Montanide ISA 51.

### Example 3: Immunogenicity and Cytotoxicity Data of CoVepiT

A) CTL Immunogenicity Evaluation after CoVepiT Immunization in Different HLA-A2+ Transgenic Mouse Strains

CoVepiT immunogenicity was evaluated in different strains of HLA transgenic mice (FIG. 6). For this study, CoVepiT (12 selected CTL peptides+HTL pan DR) was prepared by an emulsification protocol as per as method used for drug manufacture and subcutaneously injected into mice on Day 0 and Day 14.

Different HLA-A2 Strain were Tested:

(Group 1) B6.Cg-Immp2ITg (HLA-A2/H2D) 2Enge/J mice mice. (n=16, age 6-7 weeks); 8 mice were immunized (4 males, 4 females) and 8 naïve mice were used as negative control (4 males, 4 females).

(Group 2) HLA-A2/HLA-DR1+ double transgenic mice (strain from Pasteur institute, Pascolo S, Lemonnier FA, JEM 1997) (n=39 mice age 10-48 weeks), 27 mice were immunized and 12 naïve mice were used as negative control. 3 mice were pooled for each IFN-γ analysis.

(Group 3) HLA-A2/H2kB transgenic mice (CB6F1-Tg(HLA-A*0201/H2-Kb)A*0201; Taconic stock #9659) (n=7 mice age 5-6 weeks, males), 4 mice were immunized and 3 naïve mice were used as negative control).

T cell effector responses was determined by measuring IFN-γ production by CD8+ T cells compared to naïve mice (non-vaccinated mice) after in vitro restimulation for 24 hours with the pool of 12 corresponding wild-type peptides. As shown on FIG. 6, the 3 different HLA-A2 transgenic mice strains illustrated a strong IFN-γ CD8 T cells response after immunization with CoVepiT vaccine, all mice of each strain responded to the stimulation showing the reliability and reproducibility of the immunization experiments. As in vitro restimulation was performed with wild-type peptides, the data also show that elicited T cell response is cross-reactive with the SARS-COV2 virus sequence.

Because PADRE binds with high affinity to human HLA-DR types, and with moderate affinity to mouse I-A^{b/d} MHC haplotypes, the use of HLA-A2 and HLA-DR1 double transgenic mice allow to study the impact of PADRE in a more relevant model for CD4+ T cell responses which indirectly impact the quality of CD8+ T cell responses. Data show that CoVepiT immunization in HLA-A2/DR1 mice is highly effective in this specific HLA-A2/DR1+ double transgenic model mimicking the human situation (HLA ABC and HLA DR).

### B) Cytotoxic Activity Measured by Granzyme B Secretion and T2 Cell Model:

Cytotoxic T lymphocytes (CTLs) are controlling intracellular pathogens by recognizing and clearing infected viral target cells. Experimental methods were implemented, to estimate the CTL's efficacy in detecting granzyme B protease inducing target cell death and direct killing assay.

### Granzyme B Model:

To demonstrate that specific CD8 T cells generated after immunization by CoVepiT achieve cytotoxic activity against SARS-COV2 infected cells, Granzyme B secretion and cytolytic activity of CD8⁺ T cells were measured after in vitro restimulation with SARS-COV2 peptides-presenting HLA-A2⁺ human cells.

Granzyme B is established as a caspase-like serine protease that is released by cytotoxic lymphocytes to kill virus-infected cells.

For this study, HLA-A2/H2 KB transgenic mice (n=7, 7-28 weeks old males) were immunized with 12 CTL peptides of CoVepiT+pan-HLA-DR PADRE peptide emulsified in Montanide ISA 51 (50 μg of each peptide+25 μg of HTL peptide). On Day 10, spleen and draining lymph nodes were collected. CD8⁺ T cells were sorted. To measure secretion of granzyme B, CD8⁺ T cells were in vitro restimulated with wild-peptide 14/23/48/19 (SEQ ID NOs: 14, 23, 48 and 19, respectively) encoding for Spike/Receptor Binding Domain/Membrane/Nucleocapsid (S/RBD/M/N) SARS COV-2 proteins (10 μg/mL each) plus CD8⁻ T cells (ratio 1:1) (0.1×10⁶CD8⁺ T cells) to present peptides. As demonstrated in FIG. 7, a specific Granzyme B response is observed after restimulation with the pool of 4 wild type peptides from Spike/Receptor Binding Domain/Membrane/Nucleocapsid (S/RBD/M/N) compared to no peptide stimulation (medium) showing that CoVepiT vaccination induces Granzyme B+CD8+ T cell populations with high cytotoxicity potential.

### T2 Cells Model Presenting Viral Proteins at the Surface:

Cytotoxic activity of CD8+ T Cells obtained after CoVepiT vaccination, was also measured by Chromium 51 release assay in HLA-A2 T2 human cells presenting 4 viral proteins. This second method was employed to confirm direct cytolytic capacity of CD8+ T cells against SARS-COV2 infected target cells using chromium 51 release assay. To mimic SARS-COV2 infection and viral peptides presentation by HLA-A2 cells, HLA-A2+T2 human cells were pulsed overnight with the same wild-type peptides from viral proteins Spike/Receptor Binding Domain/Membrane/Nucleocapsid (48/19/14/23 S/RBD/M/N, SEQ ID NOs 48, 19, 14 and 23 respectively) and used as target cells to measure cytotoxic functions of elicited CD8+ T cells after vaccination. Prior the assay, CD8 T cells isolated from immunized mice were expanded in vitro with peptide vaccine 14/23/48/19 (SEQ ID NOs 48, 19, 14 and 23 respectively) (2 μg/mL each) plus cytokine (IL-7, IL-21 and IL-2) to increase the pool of viral-specific T cells. T2 cells were labeled with chromium 51 then cocultured with CD8 T cells at ratio 40:1 or 15:1 for 4 hours. Chromium released by target T2 cells was counted in the supernatant using a gamma counter to quantify specific cytolysis. FIG. 8 shows a specific lysis of the SARS-COV2 peptide-presenting T2 cells compared with unpulsed T2 cells at high (40:1) or low (15:1) Effector: Target ratio.

Altogether, these immunogenicity and cytotoxicity data indicate that CoVepiT elicits SARS-COV2 specific Th1-biased CTL cells, producing IFNg and Granzyme B cytolytic granules. These immunogenicity results were more pronounced where the bi-transgenic model was expressing both HLA A2 and HLA DR. Finally, the CTL cells induced by the CoVepiT vaccine are capable to recognize and kill SARS-COV2 peptides-presenting human cells in a direct manner.

### Example 4: Dose Escalation—One or Two Injections—Cellular Immunogenicity Evaluation, No Humoral Responses in HLA A2.1 Model

### A) Dose and Schedule (One or Two Injections) Testing in HLA-A2.1 Model

This experiment was conducted using 56 transgenic mice (male, age 6-7 weeks) HLA-A/H2-D. Experimental design is detailed in the table below. Nine different groups were studied for pharmacology and immunogenicity analysis with 3 escalating doses and one or two injections schedule studied.

| | | | Dose | Dosing | Dosing | |
|---|---|---|---|---|---|---|
| Group | N | Treatment | (μg/peptide) | Route | Time Point | Necropsy |
| 1A | 4M | Covepit ® | 1 | SC | Day 0 | Day 14 |
| 1B | 4M | Covepit ® | 5 | SC | Day 0 | Day 14 |
| 1C | 4M | Covepit ® | 50 | SC | Day 0 | Day 14 |
| | 4F | | | | | |
| 2A | 4M | Covepit ® | 1 | SC | Day 0 and 14 | Day 21 |
| 2B | 4M | Covepit ® | 5 | SC | Day 0 and 14 | Day 21 |
| 2C | 4M | Covepit ® | 50 | SC | Day 0 and 14 | Day 21 |
| | 4F | | | | | |
| 3A | 4M | Naive | N/A | N/A | N/A | Day 0 |
| | 4F | | | | | |
| 4A | 4M | Montanide | N/A | SC | Day 0 | Day 14 |
| | 4F | | | | | |
| 4B | 4M | Montanide | N/A | SC | Day 0 and 14 | Day 21 |
| | 4F | | | | | |

SC: subcutaneous

One or 2 administrations of CoVepiT vaccine were also compared (group 1 versus group 2). The product was prepared with 12 CTL peptides plus Pan DR HTL epitope emulsified with the adjuvant Montanide ISA 51 (1/1 m/m) to be injected by subcutaneous route (100 uL). In parallel, groups of Naïve mice (group 3A) or mice injected with adjuvant only (group 4A and 4B) were used as negative control for immunization.

For pharmacology measurement, on Day 14 necropsy (one injection at DO) or Day 21 necropsy (2 injections D0-D14), spleen, Inguinal, axillary, and brachial lymph nodes were harvested and processed for single suspension to isolate CD8+ T cells from each individual mouse. CD8 T cells were sorted using negative sorting Macs Miltenyi microbeads and restimulated in vitro (0.3×10⁶ cells) with of the pool of 12 corresponding wild-type peptides (10 μg/mL each) to evaluate cross-reactivity of elicited T cell response towards SARS-COV2 virus antigens.

The naïve mice group elicits few immunogenicity and serve as negative control whereas all treated animals treated with CoVepiT showed strong immunogenicity response.

In this experiment in HLA-A/H2-D transgenic mice, it was not observed significative impact, or correlation between 1 or 2 administrations related to immunogenicity, as shown in FIG. 9. One or two injections of CoVepiT elicit a similar high IFN-γ response in this mouse model HLA-A2.1⁺.

Considering the different dose tested, the 5 μg dose (5 μg/each CTL peptide) induced better immunogenicity, the maximal effect was observed after a single injection. However, no difference was observed between 1, 5 or 50 μg after a second injection providing the same good level of immunogenicity for the 3 doses groups. Altogether, the intermediate dose (5 μg/each CTL peptide) is likely to be efficient to induce strong CD8 T cell immunogenicity. The highest dose (50 μg/each CTL peptide) gives also a strong immunogenicity validating the dose for the pharmaco-toxicological model.

Increasing doses or two injections are not providing increased of immunogenicity in this Transgenic model HLA-A/H2-D.

### B) Immunogenicity Study in HLA-A2/DR1 Mice

HLA-A2/HLA DR1 double transgenic mice were used for the study n=30 mice (aged 10-12 weeks).

The HLA-A2.1/HLA-DR1 double transgenic model was selected to explore at the highest dose (50 μg/each CTL peptide), the schedule of one injection versus two injections following protocol described in FIG. 10. In this double transgenic model, one administration was again sufficient to induce significant immunogenicity versus the naïve mice group.

A higher immunogenicity response was observed after the second administration of the vaccine versus the One injection group in the HLA A2-DR1 model.

A significant higher immunogenicity response was observed after the second administration of the vaccine versus the One injection group in the HLA A2−/HLADR1 model.

### C) Humoral Responses Analysis

Serum of immunized mice for this part of the study corresponds to the group 1C, 2C and 3A N=24 mice (12 females and 12 males, age 6-7 weeks).

SARS-COV2 specific antibody was also quantified in the sera of immunized mice to determine whether the vaccine can promote humoral B cell response. HLA-A/H2-D transgenic mice were subcutaneous injected with CoVepiT (12 peptides 50 μg plus HTL 25 μg emulsified in Montanide) (Group 1C and 2C detailed in the table). Sera from Day 14 (one injection) or Day 21 (2 injection) were collected and antibody specific for spike was quantified in the sera by ELISA. For this test, Spike or RBD recombinant proteins were immobilized on the plate (10 μg/mL) then sera were added at serial dilutions. Revelation of Mouse IgG and IgA performed using a polyclonal peroxidase labeled antibody and Tetramethylbenzidine substrate. A positive serum of mice containing specific anti spike and RBD antibodies was used as positive control for the detection. Data in FIG. 11 demonstrate that no antibody specific for spike and RBD protein was detected in all serum tested after immunization of mice with 1 or 2 doses (50 μg of each peptide). These mice were well immunized since illustrated at necropsy significant IFNγ ELISPOT response FIG. 9 (dose 50 μg one or two administrations).

Altogether these data in HLA A2-DR+ transgenic model demonstrate that CoVepiT vaccine elicits strong Th1-biased (IFNg+) CD8 T cell responses against SARS-CoV-2 antigens and with a significant higher immunogenicity response observed after the second administration of the vaccine.

CoVepiT as T multiepitope vaccine (epitopes selected from 11 proteins of SARS-CoV 2 including Spike and RBD protein) induced a strong T cellular response and this T cellular vaccine is not eliciting humoral response versus the Spike or RBD proteins.

### Example 5: Lung Resident Memory T Cell Immunogenicity in Old Versus Young Mice

HLA-A2/HLADR1 double transgenic model was used for this experiment (30 young animals (10-12 weeks)-30 aged animals (10-12 months). N=24 young mice and N=36 aged mice were immunized with CoVepiT; n=6 young and n=6 aged mice were naïve (no vaccination).

SARS-Cov2 multiepitope vaccine (CoVepiT) was evaluated in vivo in HLA-A2/DR1 double transgenic in young mice (12 weeks) versus aged mice (10-12 months) in order to evaluated CoVepiT response in immunosenescent situations. The age of 10-12 months is the oldest age that could be tested for HLA-A2/DR1 transgenic mice since this transgenic strain has a short-life expectancy (1 year).

As detailed in the FIG. 12A, 1 or 2 injections of the 12 CTL peptides plus the Pan DR HTL epitope were performed. The highest dose of 50 μg of each CTL peptide plus 25 μg of PADRE was emulsified with Montanide 15A51(1/1 m/m) and subcutaneously injected into the mice (100 uL). In parallel, 2 groups of young and old naïve mice were used as negative control for the experiment. On Day 10 or 21, spleen and lungs were harvested and mechanically dissociated to obtain a single cell suspension after lavage of lungs to remove excess of blood. Bronchoalveolar lavages (BALs) were also performed to collect T cells into the respiratory track. The organs of 3 young or aged mice were pooled leading to 4 independent analysis for young mice group and 5 independent analysis for aged mice group.

T cell immunogenicity in young and aged mice was evaluated after in vitro stimulation with SARS-COV2 wild type peptides for 24 hours. IFNγ response was quantified by ELISPOT on Day 14 or Day 21 following vaccination; CD8+ T cells mixed with CD8− cells pulsed with peptides (10 μg/mL each). No peptide stimulation (Medium) was used as basal IFN-γ aspecific secretion. Similar IFNγ response was observed after CoVepiT immunization of young and aged mice illustrating the efficacy of this vaccine in immunosenescent situation (FIG. 12B). T cell response was not only observed in the secondary lymphoid organ since a robust IFN-γ secretion was obtained with T cells isolated from the lung and Bronchoalveolar Lavage (BAL) of both young and aged mice after restimulation with all 12 wild type peptides with again similar response between, young and aged mice in term of elicited immunogenicity in the lung and respiratory tract (FIG. 12C, D). To investigate whether T cell are specific of all proteins targeted by the CoVepiT vaccine, T cells were restimulated with different pool of peptides corresponding to pool #1 Spike/RBD/nsp3, pool #2 M/N/ORf3, and pool #3 nsp 4/5/12/13/14/16 (FIG. 12E). T cells responded to all stimulation with similar magnitude demonstrating that vaccine induce broad immunogenicity against the different SARS-COV2 protein in periphery and locally into the lung tissue and without statistical difference between young and aged animals. This data also illustrates that no peptide immunodominant response was observed after vaccination.

Altogether, the data demonstrate that CoVepiT vaccine elicits CTL immunogenicity in periphery as well as in the lung and respiratory tract with similar magnitude response in young and aged immunosenescent mice and confirm the immunogenicity all SARS-COV2 peptides without immunodominance of a peptide.

Tetramer analysis by Flow cytometry were further performed as one of the available research tools to help to further characterize elicited T cell phenotype. Parenchyma resident T cells and circulating T cells into the lung were discriminated in this test with the CD8α/CD8β staining. Anti-CD8α APCe670 antibody was injected intravenously few minutes prior sacrifice of the mice, this method allows the staining of only circulating CD8 T cells with the Anti-CD8α APCe670 antibody. Tissue resident lung T cell were characterized by CD8α−/6+ phenotype (FIG. 13A gating strategy). A high frequency of viral specific Tet+ T cells both in the spleen and the lung parenchyma was detected in immunized mice (FIGS. 13B and C). Two administrations of 50 μg of the CoVepiT vaccine, significantly increased the frequency of viral lung tissue resident T cells in aged mice and in periphery in young and aged mice (FIG. 13C). Tetramer-positive T cell phenotype characterization shows that a large quantity of lung CD8+ T cells express T resident memory markers including CD103 and/or CD49a in both young and aged mice groups. The majority of CD8 lung T cells (^{˜}60%) also express CXCR6 chemokine receptor and to lesser extent express CXCR3 receptor (^{˜}30%) which are both related to lung specific migration.

The data demonstrate that CoVepiT vaccine elicits Tissue-resident viral specific CD8 T cells in the lung and respiratory tract of both young and aged animals constituting hence a local barrier provided by sentinel memory T cells with cytotoxic function.

### Example 6: CoVepiT Pharmaco-Toxicology Study in HLA-A2.1/H2D Transgenic Mice

Immunization experiments were conducted with the highest dose of 50 μg CTL/epitope. 40 transgenic mice were studied in 5 groups with 4 Males and 4 Females in each group (age 6-7 weeks)—the transgenic model was mice HLA-A/H2-D 2Enge/J.

Groups used in this pharmaco-toxicological study:

| | | | Dose | Dosing | Dosing | | |
|---|---|---|---|---|---|---|---|
| Group | N | Treatment | (μg/peptide) | Route | Time Point | Necropsy | Readouts |
| 1C | 4M | Covepit ® | 50 | SC | Day 0 | Day 14 | PharmacoTOX |
| | 4F | | | | | | |
| 2C | 4:M | Covepit ® | 50 | SC | Day 0 and 14 | Day 21 | PharmacoTOX |
| | 4F | | | | | | |
| 3A | 4M | Naive | N/A | N/A | N/A | Day 0 | PharmacoTOX |
| | 4F | | | | | | |
| 4A | 4M | Montanide | N/A | SC | Day 0 | Day 14 | PharmacoTOX |
| | 4F | | | | | | |
| 4B | 4M | Montanide | N/A | SC | Day 0 and 14 | Day 21 | PharmacoTOX |
| | 4F | | | | | | |

Mice were injected with 1 injection (D0, group 1C) or 2 injections (D0-D14, group 2C) of CoVepiT vaccine (50 μg of each CTL peptide+HTL peptide 25 μg). The peptides were emulsified with the adjuvant Montanide ISA 51 (1/1 m/m) and injected by subcutaneous route (100 uL) as the subcutaneous route is also intended for the phase I clinical study. In parallel, groups of Naïve mice not treated (Group 3A) and a group receiving the adjuvant Montanide ISA 51 (One or two injections, Group 4A and 4B) served as negative controls of the immunization.

### Clinical and Biological Observations:

Daily clinical observations were performed by a veterinarian. Each mouse was assessed for any visible abnormalities in the fur, skin with focus at the injection site, eyes, respiration, behavior, etc. The cage was also observed for signs of food and water intake, and urination and defecation. Mice were also weighted weekly using a countertop scale.

No evidence of local irritation at the injection sites, behavior, morbidity, or mortality was observed following injection of CoVepiT vaccine regardless of the doses (1 μg, 5 μg and 50 μg) tested.

After one injection, the follow up of clinical observation was done every day (from D0 to D14) and was qualified as normal in all groups treated.

After two injections, the follow up every day (from D11 to D21) reported the same clinical observation for all mice. Only a hair loss was observed in 2 mice treated with CoVepiT (one hair loss observation at 1 μg and at 50 μg), one hair loss observation was also observed in the group treated with the adjuvant Montanide alone. Such observations were considered common in mice and are frequently correlated with stress and environmental modifications.

No difference in the weight (FIG. 14) was neither observed following vaccination confirming good tolerance profile in these transgenic mice.

### Hematology and Blood Chemistry Study:

For this study, the highest dose of 50 μg/peptide was selected to evaluate the potential toxicity in transgenic mice model as this model was established to elicit expected immunogenicity responses to the vaccine. Covepit was administered one or 2 times as detailed in the Table (Group 1C and 2 C).

Blood was collected on Day 2 and Day 10 for the one injection groups or Day 14 and Day 21 for the two injections groups. In parallel, naïve mice receiving no treatment and mice treated with adjuvant (emulsion only with Montanide ISA51) one or 2 injections were used as control. Mice were sacrificed on Day 0 (naïve mice), Day 14 (one injection) or Day 21 (2 injections)

Clinical chemistry parameters were including Albumine, Alkaline phosphatase, Creatine kinase, Lactate deshydrogenase, Alanine aminotransferase, Aspartate Aminotransferase, Sodium, Potassium, Chloride analysed in (FIG. 15).

No specific biological change was observed in the treated groups with the test item CoVepiT at 50μg versus the control group (naïve not treated mice) or the adjuvant. It was not observed notable elements with one or two injections of CoVepiT compared to the adjuvant group (receiving one or two injections) considering albumin, Alkaline phosphatase, CK, LDH, AST, ALT, Sodium, Potassium, Chloride.

In parallel, White blood cell count, Red blood cell count, Hemoglobin, hematocrites, platelets, were also counted (FIG. 16).

Hematological parameters were not substantially modified in the treated groups with the test item CoVepiT at 50μg versus the control group (naïve not treated mice). It was not observed notable elements with one injection of CoVepiT compared to the adjuvant group (receiving one injection) considering the blood cells counts: white blood cell count, Red Blood cell count, Hemoglobulin, hematocrit, platelets; lymphocytes, neutrophiles, monocytes and eosinophiles.

With two injections, no significant changes were observed, except a slight increase of monocytes and a slight decrease of lymphocytes population after the two CoVepiT vaccination versus the naïve group and the Montanide group (also receiving two injections) on Day 21.

### Example 7: Ex Vivo T-Cell Epitope Reactivity in Asymptomatic and Convalescent Covid-19 Individuals and Unexposed Donors

The strong immunogenicity of the selected peptides and the recognition by memory T Cells was confirmed in convalescent COVID-19 Patients (Erreur ! Source du renvoi introuvable.) with the same responses (quantity and intensity) observed between HLA-A2+ and HLA-A2− donors.

The ex vivo clinical update in 88 COVID 19 convalescent patients confirms the immunogenicity of the 12 CTL selected peptides on T cells both in asymptomatic and in hospitalized (moderate to severe) COVID-19 convalescent patients, whatever their HLA-A2 status (negative or positive).

### Example 8: CoVepiT Promotes Long Term T Cell Immunogenicity

CTL long term Immunogenicity was evaluated after one or two administrations of CoVepiT vaccine in HLA-A2 transgenic mice, a total of 24 mice were vaccinated (n=12 one injection, and n=12 two injection) and 6 naïve mice (non-immunized) were used as negative control for the experiment. A dose of 50 ug of each peptide+25 ug HTL emulsified in Montanide ISA51 (1/1 m/m) was subcutaneously injected in the mice, the schedule of injections is described in FIG. 18A.

Long-term T cell immunogenicity was assessed on Day 60 (1 administration) or on Day 74 (2 administrations) after ex vivo restimulation. CD8⁺ T cells were isolated from the spleen and T cells were isolated from the lung or the Bronchoalveaolar lavage (BAL) of the immunized and naïve mice. In each group, spleen, BAL, and lung of 3 mice were pooled for analysis. IFN-γ response was quantified by ELISPOT after ex vivo restimulation with 12 wild-type corresponding peptides (10 ug/mL peptide) or without peptide (Medium) to measure basal IFN-γ aspecific secretion.

CoVepiT elicits a high long-term CTL immunogenicity in the spleen 2 months after a single or double vaccination FIG. 18B. A significant IFN-γ response was also observed in the lung and the respiratory tract (BAL) on Day 74 following 2 administrations of CoVepiT Vaccine. This data illustrate that CoVepit vaccine promotes a persistent memory T cell response specific of the SARS-COV2 virus in periphery and, also locally into the lung. Two injections of the vaccine induced the highest long-term response.

### Summary of Preclinical In Vivo Data

A dose effect was not explicit in the preclinical data tested. CoVepiT injected at a dose of 1, 5 or 50 μg of each peptide showed high immunogenicity in vivo (IFNg secretion by CD8+ T cells). The highest dose given once or twice gives good immunogenicity level in HLA transgenic mice mimicking human HLA system (HLA-A2/H2D model).
In the HLA-A2/H2D model, one or two administrations of CoVepiT at the highest dose elicit a similar a immunogenicity, whereas in HLA-A2/HLA DR1 double transgenic mice two injections at the highest dose induced higher immunogenicity compared to one administration.
A good safety profile was obtained after vaccination. In all in vivo model tested, CoVepiT vaccine was well tolerated without any adverse effect. Hematological analysis, chemistry and histopathological analyses showed no major difference between CoVepiT vaccinated group versus naïve mice or mice injected with adjuvant only. Inflammation was noted at the injection site, the same level of inflammation was observed with the adjuvant alone.
The vaccine stimulates T cells defenses without eliciting humoral response (anti-Spike antibodies).
CoVepiT vaccine induced with similar magnitude immunogenicity in young and immunosenescent aged mice.
SARS-COV2 specific T cells generated after immunization have cytotoxic capacity to directly and specifically kill SARS-COV2 peptides-presenting human cells and produced cytolytic granules to eliminate viral infected cells.
CoVepiT elicits T cell local CD8+ T cell immunity in both young and aged mice since SARS-COV2 specific T cells were quantified in the lung after vaccination expressing hallmarks or resident memory T cells (Trm).
CoVepit vaccine promotes a persistent memory T cell response specific of the SARS-COV2 virus in periphery and, also locally into the lung.

### A/ First Items of the Disclosure

Item 1—A vaccine composition comprising one or several peptides (CTL peptide) inducing a CTL response against a SARS-CoV protein and optionally one or several peptides (BCL peptide) inducing a B cell response against SARS-CoV protein and optionally one or several peptides (HTL peptide) inducing a T helper response.
Item 2—The vaccine composition of item 1, wherein the composition comprises
At least 1, preferably at least 5, CTL epitopes or neo-epitopes;
Optionally at least 1, preferably at least 2, notably 2 to 8, BCL peptides/epitopes i; and
Optionally at least 1 HTL peptide/epitope.
   Item 3—The vaccine composition of item 1 or 2, wherein the composition comprises
At least 1, preferably at least 5, CTL epitopes or neo-epitopes;
at least 1, preferably at least 2, notably 2 to 8, BCL peptides/epitopes; and
at least 1 HTL peptide/epitope.
   Item 4—The vaccine composition of item 1 or 2, wherein the composition comprises
At least 1, preferably at least 5, CTL epitopes or neo-epitopes;
at least 1 HTL peptide/epitope.
   Item 5—The vaccine composition of item 1 or 2, wherein the composition comprises
at least 1, preferably at least 2, notably 2 to 8, BCL peptides/epitopes; and
at least 1 HTL peptide/epitope.
   Item 6—The vaccine composition of item 1 or 2, wherein the composition comprises
At least 1, preferably at least 5, CTL epitopes or neo-epitopes;
at least 1, preferably at least 2, notably 2 to 8, BCL peptides/epitopes i; and
   Item 7—The vaccine composition of item 1 or 2, wherein the composition is a combination of two vaccine compositions, a first composition comprising at least 1, preferably at least 5, CTL epitopes or neo-epitopes and optionally 1 HTL peptide/epitope, and a second composition comprising at least 1, preferably at least 2, notably 2 to 8, BCL peptides/epitopes and optionally 1 HTL peptide/epitope.
   Item 8—The vaccine composition according to any one of items 1-7, wherein the vaccine composition comprises at least 1 CTL epitope and at least 1 CTL neoepitope, advantageously 1 to 15 CTL epitopes and 1 to 15 CTL neo-epitopes.
   Item 9—The vaccine composition according to any one of items 1-8, wherein the CTL epitopes are selected in Table 1.
   Item 10—The vaccine composition according to any one of items 1-9, wherein the CTL neo-epitopes are selected in Table 2 and/or 3.
   Item 11—The vaccine composition according to any one of items 1-10, wherein the BCL peptides/epitopes are selected in Table 4 or 5.
   Item 12—The vaccine composition according to any one of items 1-11, wherein the composition comprises a T helper peptide, especially PADRE (aKXVAAWTLKAAa with X and a respectively indicating cyclohexylalanine and d-alanine).
   Item 13—The vaccine composition according to any one of items 1-12, wherein the CTL epitopes or neo-epitopes of the vaccine composition target one or several proteins of SARS-CoV, especially selected in the group consisting of Spike glycoprotein (S), Nucleocapsid protein (N), Membrane glycoprotein (M) and ORfs, more particularly ORF3, ORF1Ab (nsp4), ORF1ab (nsp3), ORF1 (pp1ab), and ORF1Ab (nsp6).
   Item 14—The vaccine composition according to any one of items 1-12, wherein the CTL epitopes or neo-epitopes of the vaccine composition are selected for targeting 1, 2, 3, 4, 5, 6, 7 or 8 of Spike glycoprotein (S), Nucleocapsid protein (N), Membrane glycoprotein (M), ORF3, ORF1Ab (nsp4), ORF1ab (nsp3), ORF1 (pp1ab), and ORF1Ab (nsp6).
   Item 15—The vaccine composition according to any one of items 1-12, wherein the CTL epitopes or neo-epitopes of the vaccine composition are selected for targeting Spike glycoprotein (S) and Nucleocapsid protein (N); Spike glycoprotein (S) and Membrane glycoprotein (M); Spike glycoprotein (S) and ORF3; Spike glycoprotein (S) and ORF1Ab (nsp4); Spike glycoprotein (S) and ORF1ab (nsp3); Spike glycoprotein (S) and ORF1 (pp1ab); Spike glycoprotein (S) and ORF1Ab (nsp6); Nucleocapsid protein (N) and Membrane glycoprotein (M); Nucleocapsid protein (N) and ORF3; Nucleocapsid protein (N) and ORF1Ab (nsp4); Nucleocapsid protein (N) and ORF1ab (nsp3); Nucleocapsid protein (N) and ORF1 (pp1ab); Nucleocapsid protein (N) and ORF1Ab (nsp6); Spike glycoprotein (S), Nucleocapsid protein (N) and Membrane glycoprotein (M); Spike glycoprotein (S), Nucleocapsid protein (N) and ORF3; Spike glycoprotein (S), Nucleocapsid protein (N) and ORF1Ab (nsp4); Spike glycoprotein (S), Nucleocapsid protein (N) and ORF1ab (nsp3); Spike glycoprotein (S), Nucleocapsid protein (N) and ORF1 (pp1ab); Spike glycoprotein (S), Nucleocapsid protein (N) and ORF1Ab (nsp6); Membrane glycoprotein (M) and ORF3; Membrane glycoprotein (M) and ORF1Ab (nsp4); Membrane glycoprotein (M) and ORF1ab (nsp3); Membrane glycoprotein (M) and ORF1 (pp1ab); Membrane glycoprotein (M) and ORF1Ab (nsp6); Spike glycoprotein (S), Membrane glycoprotein (M) and ORF3; Spike glycoprotein (S), Membrane glycoprotein (M) and ORF1Ab (nsp4); Spike glycoprotein (S), Membrane glycoprotein (M) and ORF1ab (nsp3); Spike glycoprotein (S), Membrane glycoprotein (M) and ORF1 (pp1ab); Spike glycoprotein (S), Membrane glycoprotein (M) and ORF1Ab (nsp6); Nucleocapsid protein (N), Membrane glycoprotein (M) and ORF3; Nucleocapsid protein (N), Membrane glycoprotein (M) and ORF1Ab (nsp4); Nucleocapsid protein (N), Membrane glycoprotein (M) and ORF1ab (nsp3); Nucleocapsid protein (N), Membrane glycoprotein (M) and ORF1 (pp1ab); Nucleocapsid protein (N), Membrane glycoprotein (M) and ORF1Ab (nsp6); Spike glycoprotein (S), Nucleocapsid protein (N), Membrane glycoprotein (M) and ORF3; Spike glycoprotein (S), Nucleocapsid protein (N), Membrane glycoprotein (M) and ORF1Ab (nsp4); Spike glycoprotein (S), Nucleocapsid protein (N), Membrane glycoprotein (M) and ORF1ab (nsp3); Spike glycoprotein (S), Nucleocapsid protein (N), Membrane glycoprotein (M) and ORF1 (pp1ab); or Spike glycoprotein (S), Nucleocapsid protein (N), Membrane glycoprotein (M) and ORF1Ab (nsp6).
   Item 16—The vaccine composition according to any one of items 1-15, wherein for each targeted protein, the vaccine composition comprises at least one CTL epitope selected in Table 1 and at least one CTL neoepitope selected in Table 2 and/or 3.
   Item 17—The vaccine composition according to any one of items 1-15, wherein, for each targeted protein, the vaccine composition comprises at least one CTL epitope selected in Table 1 and at least two CTL neoepitopes selected in Table 2 and/or 3.
   Item 18—The vaccine composition according to any one of items 1-15, wherein, for each targeted protein, the vaccine composition comprises at least two CTL neoepitopes selected in Table 2 and/or 3.
   Item 19—The vaccine composition according to any one of items 1-15, wherein for each targeted protein, the vaccine composition independently for the different targeted proteins comprises: at least one CTL epitope selected from Table 1 and at least one CTL neoepitope selected from Table 2 and/or 3; or at least one CTL epitope and at least two CTL neoepitopes selected from Table 2 and/or 3; or at least two CTL neoepitopes selected from Table 2 and/or 3.
   Item 20—The vaccine composition according to any one of items 1-15, wherein the vaccine composition comprises CTL epitopes/neoepitopes targeting Spike glycoprotein (S) and the vaccine composition comprises:
at least one CTL epitope targeting Spike glycoprotein (S) selected from Table 1 and at least one CTL neoepitope targeting Spike glycoprotein (S) selected from Table 2 and/or 3; or
at least one CTL epitope targeting Spike glycoprotein (S) selected from Table 1 and at least two CTL neoepitopes targeting Spike glycoprotein (S) selected from Table 2 and/or 3; or
at least two CTL neoepitopes targeting Spike glycoprotein (S) selected from Table 2 and/or 3.
   Item 21—The vaccine composition according to any one of items 1-15, wherein the vaccine composition comprises CTL epitopes/neoepitopes targeting Nucleocapsid protein (N) and the vaccine composition comprises:
at least one CTL epitope targeting Nucleocapsid protein (N) selected from Table 1 and at least one CTL neoepitope targeting Nucleocapsid protein (N) selected from Table 2 and/or 3; or
at least one CTL epitope targeting Nucleocapsid protein (N) selected from Table 1 and at least two CTL neoepitopes targeting Nucleocapsid protein (N) selected from Table 2 and/or 3; or
at least two CTL neoepitopes targeting Nucleocapsid protein (N) selected from Table 2 and/or 3.
   Item 22—The vaccine composition according to any one of items 1-15, wherein the vaccine composition comprises CTL epitopes/neoepitopes targeting Membrane glycoprotein (M) and the vaccine composition comprises:
at least one CTL epitope targeting Membrane glycoprotein (M) selected from Table 1 and at least one CTL neoepitope targeting Membrane glycoprotein (M) selected from Table 2 and/or 3; or
at least one CTL epitope targeting Membrane glycoprotein (M) selected from Table 1 and at least two CTL neoepitopes targeting Membrane glycoprotein (M) selected from Table 2 and/or 3; or
at least two CTL neoepitopes targeting Membrane glycoprotein (M) selected from Table 2 and/or 3.
   Item 23—The vaccine composition according to any one of items 1-15, wherein the vaccine composition comprises CTL epitopes/neoepitopes targeting at least one ORF selected from the group consisting of ORF3, ORF1Ab (nsp4), ORF1ab (nsp3), ORF1 (pp1ab), and ORF1Ab (nsp6), and the vaccine composition comprises:
at least one CTL epitope targeting at least one ORF selected from the group consisting of ORF3, ORF1Ab (nsp4), ORF1ab (nsp3), ORF1 (pp1ab), and ORF1Ab (nsp6) and selected from Table 1 and at least one CTL neoepitope targeting at least one ORF selected from the group consisting of ORF3, ORF1Ab (nsp4), ORF1ab (nsp3), ORF1 (pp1ab), and ORF1Ab (nsp6) and selected from Table 2 and/or 3; or
at least one CTL epitope and at least two CTL neoepitopes targeting at least one ORF selected from the group consisting of ORF3, ORF1Ab (nsp4), ORF1ab (nsp3), ORF1 (pp1ab), and ORF1Ab (nsp6) and selected from Table 2 and/or 3; or
at least two CTL neoepitopes targeting at least one ORF selected from the group consisting of ORF3, ORF1Ab (nsp4), ORF1ab (nsp3), ORF1 (pp1ab), and ORF1Ab (nsp6) and selected from Table 2 and/or 3.
   Item 24—A vaccine composition as defined in any one of the items 1-23 for use for preventing or treating an infection by a severe acute respiratory syndrome-related coronavirus (SARS-CoV).
   Item 25—The vaccine composition for use according to items 24, wherein the SARS-CoV is selected from the group consisting of SARS-CoV1, SARS-CoV2 or MERS-CoV virus.
   Item 26—The vaccine composition for use according to item 24 or 25, for use for preventing or treating Covid-19.
   Item 27—The vaccine composition for use according to any one of items 24-26, wherein the subject to be treated is a subject aged 65 years or older, a subject having a cancer or having had a cancer, a subject being obese (In particular with severe obesity (body mass index [BMI] of 40 or higher [CDC-HCSP BMI>30]), a subject being diabetic, a subject having a hypertension, a subject having a sarcoidosis, a subject being immunocompromised, a subject who lives in a nursing home or long-term care facility, a subject with chronic lung disease or moderate to severe asthma, lung fibrosis, a subject who has serious heart conditions, a subject with chronic kidney disease undergoing dialysis and/or a subject with liver diseases.
   Item 28—The vaccine composition for use according to any one of items 24-27, wherein the subject is HLA-A2.

### B/ Second Items of the Disclosure

Item 1—A vaccine composition comprising one or several peptides selected from one or several peptides (CTL peptide) inducing a CTL response against a SARS-CoV protein, one or several peptides (BCL peptide) inducing a B cell response against SARS-CoV protein and one or several peptides (HTL peptide) inducing a T helper response, wherein the composition comprises:
   At least 1, 2, 3, 4 or 5 CTL epitopes selected from the Table 1 or neo-epitopes selected from the Tables 2 and/or 3;
   at least 1, 2, 3, 4 or 5 BCL peptides/epitopes selected from the Tables 4 and/or 5; and
   at least 1 HTL peptide/epitope;
or
   At least 1, 2, 3, 4 or 5 CTL neo-epitopes selected from the Tables 2 and/or 3, and optionally at least 1, 2, 3, 4 or 5 CTL epitopes selected from the Table 1; and
   at least 1 HTL peptide/epitope;
or
   at least 2, 3, 4 or 5 BCL peptides/epitopes selected from the Tables 4 and/or 5; and
   at least 1 HTL peptide/epitope;
or
   At least 1, 2, 3, 4 or 5 CTL epitopes selected from the Table 1 or neo-epitopes selected from the Tables 2 and/or 3; and
   at least 1, 2, 3, 4 or 5 BCL peptides/epitopes selected from the Tables 4 and/or 5.
Item 2—The vaccine composition of item 1, wherein the composition is a combination of two vaccine compositions, a first composition comprising
   At least 1, 2, 3, 4 or 5 CTL epitopes selected from the Table 1 or neo-epitopes selected from the Tables 2 and/or 3; and
   Optionally at least 1 HTL peptide/epitope;
   and a second composition comprising
   at least 2, 3, 4 or 5 BCL peptides/epitopes selected from the Tables 4 and/or 5; and
   at least 1 HTL peptide/epitope.
Item 3—The vaccine composition according to any one of items 1-2, wherein the vaccine composition comprises at least 1 CTL epitope and at least 1 CTL neoepitope, advantageously 1 to 15 CTL epitopes and 1 to 15 CTL neo-epitopes.
Item 4—The vaccine composition according to any one of items 1-3, wherein the composition comprises a T helper peptide, especially PADRE (aKXVAAWTLKAAa with X and a respectively indicating cyclohexylalanine and d-alanine).
Item 5—The vaccine composition according to any one of items 1-4, wherein the CTL epitopes or neo-epitopes of the vaccine composition target one or several proteins of SARS-CoV, especially selected in the group consisting of Spike glycoprotein (S), Nucleocapsid protein (N), Membrane glycoprotein (M) and ORfs, more particularly ORF3, ORF1Ab (nsp4), ORF1ab (nsp3), ORF1 (pp1ab), and ORF1Ab (nsp6).
Item 6—The vaccine composition according to any one of claims 1-5, wherein the CTL epitopes or neo-epitopes of the vaccine composition are selected for targeting 1, 2, 3, 4, 5, 6, 7 or 8 of Spike glycoprotein (S), Nucleocapsid protein (N), Membrane glycoprotein (M), ORF3, ORF1Ab (nsp4), ORF1ab (nsp3), ORF1 (pp1ab), and ORF1Ab (nsp6).
Item 7—The vaccine composition according to any one of items 1-6, wherein the CTL epitopes or neo-epitopes of the vaccine composition are selected for targeting Spike glycoprotein (S) and Nucleocapsid protein (N); Spike glycoprotein (S) and Membrane glycoprotein (M); Spike glycoprotein (S) and ORF3; Spike glycoprotein (S) and ORF1Ab (nsp4); Spike glycoprotein (S) and ORF1ab (nsp3); Spike glycoprotein (S) and ORF1 (pp1ab); Spike glycoprotein (S) and ORF1Ab (nsp6); Nucleocapsid protein (N) and Membrane glycoprotein (M); Nucleocapsid protein (N) and ORF3; Nucleocapsid protein (N) and ORF1Ab (nsp4); Nucleocapsid protein (N) and ORF1ab (nsp3); Nucleocapsid protein (N) and ORF1 (pp1ab); Nucleocapsid protein (N) and ORF1Ab (nsp6); Spike glycoprotein (S), Nucleocapsid protein (N) and Membrane glycoprotein (M); Spike glycoprotein (S), Nucleocapsid protein (N) and ORF3; Spike glycoprotein (S), Nucleocapsid protein (N) and ORF1Ab (nsp4); Spike glycoprotein (S), Nucleocapsid protein (N) and ORF1ab (nsp3); Spike glycoprotein (S), Nucleocapsid protein (N) and ORF1 (pp1ab); Spike glycoprotein (S), Nucleocapsid protein (N) and ORF1Ab (nsp6); Membrane glycoprotein (M) and ORF3; Membrane glycoprotein (M) and ORF1Ab (nsp4); Membrane glycoprotein (M) and ORF1ab (nsp3); Membrane glycoprotein (M) and ORF1 (pp1ab); Membrane glycoprotein (M) and ORF1Ab (nsp6); Spike glycoprotein (S), Membrane glycoprotein (M) and ORF3; Spike glycoprotein (S), Membrane glycoprotein (M) and ORF1Ab (nsp4); Spike glycoprotein (S), Membrane glycoprotein (M) and ORF1ab (nsp3); Spike glycoprotein (S), Membrane glycoprotein (M) and ORF1 (pp1ab); Spike glycoprotein (S), Membrane glycoprotein (M) and ORF1Ab (nsp6); Nucleocapsid protein (N), Membrane glycoprotein (M) and ORF3; Nucleocapsid protein (N), Membrane glycoprotein (M) and ORF1Ab (nsp4); Nucleocapsid protein (N), Membrane glycoprotein (M) and ORF1ab (nsp3); Nucleocapsid protein (N), Membrane glycoprotein (M) and ORF1 (pp1ab); Nucleocapsid protein (N), Membrane glycoprotein (M) and ORF1Ab (nsp6); Spike glycoprotein (S), Nucleocapsid protein (N), Membrane glycoprotein (M) and ORF3; Spike glycoprotein (S), Nucleocapsid protein (N), Membrane glycoprotein (M) and ORF1Ab (nsp4); Spike glycoprotein (S), Nucleocapsid protein (N), Membrane glycoprotein (M) and ORF1ab (nsp3); Spike glycoprotein (S), Nucleocapsid protein (N), Membrane glycoprotein (M) and ORF1 (pp1ab); or Spike glycoprotein (S), Nucleocapsid protein (N), Membrane glycoprotein (M) and ORF1Ab (nsp6).
Item 8—The vaccine composition according to any one of items 1-7, wherein for each targeted protein, the vaccine composition comprises at least one CTL epitope selected in Table 1 and at least one CTL neoepitope selected in Table 2 and/or 3; or at least one CTL epitope selected in Table 1 and at least two CTL neoepitopes selected in Table 2 and/or 3; or at least two CTL neoepitopes selected in Table 2 and/or 3.
Item 9—The vaccine composition according to any one of items 1-8, wherein the vaccine composition comprises CTL epitopes/neoepitopes targeting Spike glycoprotein (S) and the vaccine composition comprises:
   at least one CTL epitope targeting Spike glycoprotein (S) selected from Table 1 and at least one CTL neoepitope targeting Spike glycoprotein (S) selected from Table 2 and/or 3; or
   at least one CTL epitope targeting Spike glycoprotein (S) selected from Table 1 and at least two CTL neoepitopes targeting Spike glycoprotein (S) selected from Table 2 and/or 3; or
   at least two CTL neoepitopes targeting Spike glycoprotein (S) selected from Table 2 and/or 3.
Item 10—The vaccine composition according to any one of items 1-8, wherein the vaccine composition comprises CTL epitopes/neoepitopes targeting Nucleocapsid protein (N) and the vaccine composition comprises:
   at least one CTL epitope targeting Nucleocapsid protein (N) selected from Table 1 and at least one CTL neoepitope targeting Nucleocapsid protein (N) selected from Table 2 and/or 3; or
   at least one CTL epitope targeting Nucleocapsid protein (N) selected from Table 1 and at least two CTL neoepitopes targeting Nucleocapsid protein (N) selected from Table 2 and/or 3; or
   at least two CTL neoepitopes targeting Nucleocapsid protein (N) selected from Table 2 and/or 3.
Item 11—The vaccine composition according to any one of items 1-8, wherein the vaccine composition comprises CTL epitopes/neoepitopes targeting Membrane glycoprotein (M) and the vaccine composition comprises:
   at least one CTL epitope targeting Membrane glycoprotein (M) selected from Table 1 and at least one CTL neoepitope targeting Membrane glycoprotein (M) selected from Table 2 and/or 3; or
   at least one CTL epitope targeting Membrane glycoprotein (M) selected from Table 1 and at least two CTL neoepitopes targeting Membrane glycoprotein (M) selected from Table 2 and/or 3; or
   at least two CTL neoepitopes targeting Membrane glycoprotein (M) selected from Table 2 and/or 3.
Item 12—The vaccine composition according to any one of items 1-8, wherein the vaccine composition comprises CTL epitopes/neoepitopes targeting at least one ORF selected from the group consisting of ORF3, ORF1Ab (nsp4), ORF1ab (nsp3), ORF1 (pp1ab), and ORF1Ab (nsp6), and the vaccine composition comprises:
   at least one CTL epitope targeting at least one ORF selected from the group consisting of ORF3, ORF1Ab (nsp4), ORF1ab (nsp3), ORF1 (pp1ab), and ORF1Ab (nsp6) and selected from Table 1 and at least one CTL neoepitope targeting at least one ORF selected from the group consisting of ORF3, ORF1Ab (nsp4), ORF1ab (nsp3), ORF1 (pp1ab), and ORF1Ab (nsp6) and selected from Table 2 and/or 3; or
   at least one CTL epitope and at least two CTL neoepitopes targeting at least one ORF selected from the group consisting of ORF3, ORF1Ab (nsp4), ORF1ab (nsp3), ORF1 (pp1ab), and ORF1Ab (nsp6) and selected from Table 2 and/or 3; or
   at least two CTL neoepitopes targeting at least one ORF selected from the group consisting of ORF3, ORF1Ab (nsp4), ORF1ab (nsp3), ORF1 (pp1ab), and ORF1Ab (nsp6) and selected from Table 2 and/or 3.
Item 13—A vaccine composition as defined in any one of the items 1-12 for use for preventing or treating an infection by a severe acute respiratory syndrome-related coronavirus (SARS-CoV).
Item 14—The vaccine composition for use according to item 13, wherein the SARS-CoV is selected from the group consisting of SARS-CoV1, SARS-CoV2 or MERS-CoV virus, preferably SARS-CoV2.
Item 15—The vaccine composition for use according to any one of items 13-14, wherein the subject to be treated is a subject aged 65 years or older, a subject having a cancer or having had a cancer, a subject being obese (In particular with severe obesity (body mass index [BM I] of 40 or higher [CDC-HCSP BMI>30]), a subject being diabetic, a subject having a hypertension, a subject having a sarcoidosis, a subject being immunocompromised, a subject who lives in a nursing home or long-term care facility, a subject with chronic lung disease or moderate to severe asthma, lung fibrosis, a subject who has serious heart conditions, a subject with chronic kidney disease undergoing dialysis and/or a subject with liver diseases; and/or a subject being HLA-A2.

### C/ Third Items of the Disclosure

Item 1—A vaccine composition comprising one or several peptides selected from one or several peptides (CTL peptide) inducing a CTL response against a SARS-CoV protein, and optionally one or several peptides (HTL peptide) inducing a T helper response, wherein the composition comprises:
At least 1, 2, 3, 4 or 5 CTL epitopes selected from the Table 1 or neo-epitopes selected from the Tables 2 and/or 3 and wherein the CTL epitopes are selected in the group consisting of SEQ ID NOs: 3, 8, 20, 22, 23, 30, 31, 32, 33, 34, 36, 42, 48, 49, 52, 56, 59, 60, 66, 67, 70, 74, 75, 76, 77, 78, 79, 83, 84, 85, 86, 90, 91, 92, 95, 97, 101, 104, 105, 113, 120, 125, 135, 139, 140, 146 and 153; and
at least 1 HTL peptide/epitope;
or
At least 1, 2, 3, 4 or 5 CTL neo-epitopes selected from the Tables 2 and/or 3 and wherein the CTL epitopes are selected in the group consisting of SEQ ID NOs: 56, 59, 60, 66, 67, 70, 74, 75, 76, 77, 78, 79, 83, 84, 85, 86, 90, 91, 92, 95, 97, 101, 104, 105, 113, 120, 125, 135, 139, 140, 146, and 153, and optionally at least 1, 2, 3, 4 or 5 CTL epitopes selected from the Table 1 and wherein the CTL epitopes are selected in the group consisting of SEQ ID NOs: 3, 8, 20, 22, 23, 30, 31, 32, 33, 34, 36, 42, 48, 49, 52; and
optionally, at least 1 HTL peptide/epitope.
   Item 2—The vaccine composition according to item 1, wherein the composition comprises:
At least 1, 2, 3, 4 or 5 CTL epitopes selected from the Table 1 or neo-epitopes selected from the Tables 2 and/or 3 and wherein the CTL epitopes are selected in the group consisting of SEQ ID NOs: 3, 8, 22, 23, 30, 31, 32, 36, 42, 48, 52, 56, 59, 60, 66, 70, 76, 77, 78, 79, 83, 91, 92, 125, 135, 139, 140 and 146; and
at least 1 HTL peptide/epitope;
or
At least 1, 2, 3, 4 or 5 CTL neo-epitopes selected from the Tables 2 and/or 3 and wherein the CTL epitopes are selected in the group consisting of SEQ ID NOs: 56, 59, 60, 66, 70, 76, 77, 78, 79, 83, 91, 92, 125, 135, 139, 140 and 146, and optionally at least 1, 2, 3, 4 or 5 CTL epitopes selected from the Table 1 and wherein the CTL epitopes are selected in the group consisting of SEQ ID NOs: 3, 8, 22, 23, 30, 31, 32, 36, 42, 48 and 52; and
optionally, at least 1 HTL peptide/epitope.
   Item 3—The vaccine composition according to item 1, wherein the composition comprises:
At least 1, 2, 3, 4 or 5 CTL epitopes selected from the Table 1 or neo-epitopes selected from the Tables 2 and/or 3 and wherein the CTL epitopes are selected in the group consisting of SEQ ID NOs: 8, 22, 23, 31, 32, 42, 52, 66, 70, 77, 97 and 146; and
at least 1 HTL peptide/epitope;
or
At least 1, 2, 3, 4 or 5 CTL neo-epitopes selected from the Tables 2 and/or 3 and wherein the CTL epitopes are selected in the group consisting of SEQ ID NOs: 66, 70, 77, 97 and 146, and optionally at least 1, 2, 3, 4 or 5 CTL epitopes selected from the Table 1 and wherein the CTL epitopes are selected in the group consisting of SEQ ID NOs: 8, 22, 23, 31, 32, 42 and 52; and
optionally, at least 1 HTL peptide/epitope.
   Item 4—The vaccine composition according to any one of items 1-3, wherein the vaccine composition comprises:
a) at least 1 CTL epitope selected in the group consisting of SEQ ID NOs: 3, 8, 20, 22, 23, 30, 31, 32, 33, 34, 36, 42, 48, 49 and 52, and at least 1 CTL neoepitope selected in the group consisting of SEQ ID NOs: 56, 59, 60, 66, 67, 70, 74, 75, 76, 77, 78, 79, 83, 84, 85, 86, 90, 91, 92, 95, 97, 101, 104, 105, 113, 120, 125, 135, 139, 140, 146 and 153, advantageously 1 to 15 CTL epitopes and 1 to 12 CTL neo-epitopes; or
b) at least 1 CTL epitope selected in the group consisting of SEQ ID NOs: 3, 8, 22, 23, 30, 31, 32, 36, 42, 48 and 52, and at least 1 CTL neoepitope selected in the group consisting of SEQ ID NOs: 56, 59, 60, 66, 70, 76, 77, 78, 79, 83, 91, 92, 125, 135, 139, 140 and 146, advantageously 1 to 10 CTL epitopes and 1 to 10 CTL neo-epitopes; or
c) at least 1 CTL epitope selected in the group consisting of SEQ ID NOs: 8, 22, 23, 31, 32, 42 and 52, and at least 1 CTL neoepitope selected in the group consisting of SEQ ID NOs: 66, 70, 77, 97 and 146, advantageously 1 to 6 CTL epitopes and 1 to 6 CTL neo-epitopes.
   Item 5—The vaccine composition according to any one of items 1-4, wherein the composition comprises a T helper peptide, especially PADRE (aKXVAAWTLKAAa with X and a respectively indicating cyclohexylalanine and d-alanine).
   Item 6—The vaccine composition according to any one of items 1-5, wherein the CTL epitopes or neo-epitopes of the vaccine composition target one or several proteins of SARS-CoV, especially selected in the group consisting of Spike glycoprotein (5), Nucleocapsid protein (N), Membrane glycoprotein (M), Protein 3a, protein non structural (nsp) 3, 4, 6, 12, 13, 14 and 16.
   Item 7—The vaccine composition according to any one of items 1-6, wherein the CTL epitopes or neo-epitopes of the vaccine composition are selected for targeting 1, 2, 3, 4, 5, 6, 7 or 8 of Spike glycoprotein (5), Nucleocapsid protein (N), Membrane glycoprotein (M), Protein 3a, protein non structural (nsp) 3, 4, 6, 12, 13, 14 and 16.
   Item 8—The vaccine composition according to any one of items 1-7, wherein the CTL epitopes or neo-epitopes of the vaccine composition are selected for targeting one or several of the following groups
Spike glycoprotein (5) and Nucleocapsid protein (N) and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23;
Spike glycoprotein (5) and Membrane glycoprotein (M) and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitope targeting M is SEQ ID NO: 66;
Spike glycoprotein (5) and Protein 3a and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting Protein 3a are selected from one of the groups consisting of (i) SEQ ID NOs: 3, 97 and 101; and (ii) SEQ ID NO: 97;
Spike glycoprotein (5) and nsp3 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;
Spike glycoprotein (5) and nsp4 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;
Spike glycoprotein (5) and nsp6 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;
Spike glycoprotein (5) and nsp12 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting nsp12 are selected from one of the groups consisting of (i) SEQ ID NOs: 32, 33 and 153; and (ii) SEQ ID NO: 32;
Spike glycoprotein (5) and nsp13, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitopes targeting nsp13 are selected from one of the groups consisting of (i) SEQ ID NOs: 22 and 120; and (ii) SEQ ID NO: 22;
Spike glycoprotein (5) and nsp14 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitope targeting nsp14 is SEQ ID NO: 31;
Spike glycoprotein (5) and nsp16 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; and the CTL (neo)epitope targeting nsp16 is SEQ ID NO: 52;
Nucleocapsid protein (N) and Membrane glycoprotein (M) and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitope targeting M is SEQ ID NO: 66;
Nucleocapsid protein (N) and Protein 3a and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting Protein 3a are selected from one of the groups consisting of (i) SEQ ID NOs: 3, 97 and 101; and (ii) SEQ ID NO: 97;
Nucleocapsid protein (N) and nsp3 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;
Nucleocapsid protein (N) and nsp4 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;
Nucleocapsid protein (N) and nsp6 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;
Nucleocapsid protein (N) and nsp12 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp12 are selected from one of the groups consisting of (i) SEQ ID NOs: 32, 33 and 153; and (ii) SEQ ID NO: 32;
Nucleocapsid protein (N) and nsp13 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp13 are selected from one of the groups consisting of (i) SEQ ID NOs: 22 and 120; and (ii) SEQ ID NO: 22;
Nucleocapsid protein (N) and nsp14 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL epitope targeting nsp14 is SEQ ID NO: 31;
Nucleocapsid protein (N) and nsp16 and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL epitope targeting nsp16 is SEQ ID NO: 52;
Spike glycoprotein (5), Nucleocapsid protein (N) and Membrane glycoprotein (M), and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitope targeting M is SEQ ID NO: 66;
Spike glycoprotein (5), Nucleocapsid protein (N) and Protein 3a and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting Protein 3a are selected from one of the groups consisting of (i) SEQ ID NOs: 3, 97 and 101; and (ii) SEQ ID NO: 97;
Spike glycoprotein (5), Nucleocapsid protein (N) and nsp3 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;
Spike glycoprotein (5), Nucleocapsid protein (N) and nsp4 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;
Spike glycoprotein (5), Nucleocapsid protein (N) and nsp6 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;
Spike glycoprotein (5), Nucleocapsid protein (N) and nsp12 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp12 are selected from one of the groups consisting of (i) SEQ ID NOs: 32, 33 and 153; and (ii) SEQ ID NO: 32;
Spike glycoprotein (5), Nucleocapsid protein (N) and nsp13 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL (neo)epitopes targeting nsp13 are selected from one of the groups consisting of (i) SEQ ID NOs: 22 and 120; and (ii) SEQ ID NO: 22;
Spike glycoprotein (5), Nucleocapsid protein (N) and nsp14 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL epitope targeting nsp14 is SEQ ID NO: 31;
Spike glycoprotein (5), Nucleocapsid protein (N) and nsp16 and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; and the CTL epitope targeting nsp16 is SEQ ID NO: 52;
Membrane glycoprotein (M) and Protein 3a and wherein the CTL (neo)epitope targeting M is SEQ ID NO: 66; and the CTL (neo)epitopes targeting Protein 3a are selected from one of the groups consisting of (i) SEQ ID NOs: 3, 97 and 101; and (ii) SEQ ID NO: 97;
Membrane glycoprotein (M) and nsp3, and wherein the CTL (neo)epitope targeting M is SEQ ID NO: 66; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;
Membrane glycoprotein (M) and nsp4 and wherein the CTL (neo)epitope targeting M is SEQ ID NO: 66; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8; Membrane glycoprotein (M) and nsp6 and wherein the CTL (neo)epitope targeting M is SEQ ID NO: 66; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;
Membrane glycoprotein (M) and nsp12 and wherein the CTL (neo)epitope targeting M is SEQ ID NO: 66; and the CTL (neo)epitopes targeting nsp12 are selected from one of the groups consisting of (i) SEQ ID NOs: 32, 33 and 153; and (ii) SEQ ID NO: 32;
Membrane glycoprotein (M) and nsp13 and wherein the CTL (neo)epitope targeting M is SEQ ID NO: 66; and the CTL (neo)epitopes targeting nsp13 are selected from one of the groups consisting of (i) SEQ ID NOs: 22 and 120; and (ii) SEQ ID NO: 22;
Membrane glycoprotein (M) and nsp14 and wherein the CTL (neo)epitope targeting M is SEQ ID NO: 66; and the CTL epitope targeting nsp14 is SEQ ID NO: 31; Membrane glycoprotein (M) and nsp16 and wherein the CTL (neo)epitope targeting M is SEQ ID NO: 66; and the CTL epitope targeting nsp16 is SEQ ID NO: 52;
Spike glycoprotein (5), Membrane glycoprotein (M) and Protein 3a, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL (neo)epitopes targeting Protein 3a are selected from one of the groups consisting of (i) SEQ ID NOs: 3, 97 and 101; and (ii) SEQ ID NO: 97;
Spike glycoprotein (5), Membrane glycoprotein (M) and nsp3, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;
Spike glycoprotein (5), Membrane glycoprotein (M) and nsp4, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;
Spike glycoprotein (5), Membrane glycoprotein (M) and nsp6, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;
Spike glycoprotein (5), Membrane glycoprotein (M) and nsp12, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL epitope targeting M is SEQ ID NO: 66; and the CTL (neo)epitopes targeting nsp12 are selected from one of the groups consisting of (i) SEQ ID NOs: 32, 33 and 153; and (ii) SEQ ID NO: 32;
Spike glycoprotein (5), Membrane glycoprotein (M) and nsp13, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitope targeting M is SEQ ID NO: 66; and the CTL (neo)epitopes targeting nsp13 are selected from one of the groups consisting of (i) SEQ ID NOs: 22 and 120; and (ii) SEQ ID NO: 22;
Spike glycoprotein (5), Membrane glycoprotein (M) and nsp14, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL epitope targeting M is SEQ ID NO: 66; and the CTL epitope targeting nsp14 is SEQ ID NO: 31;
Spike glycoprotein (5), Membrane glycoprotein (M) and nsp16, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL epitope targeting M is SEQ ID NO: 66; and the CTL epitope targeting nsp16 is SEQ ID NO: 52;
Nucleocapsid protein (N), Membrane glycoprotein (M) and Protein 3a, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting Protein 3a are selected from one of the groups consisting of (i) SEQ ID NOs: 3, 97 and 101; and (ii) SEQ ID NO: 97;
Nucleocapsid protein (N), Membrane glycoprotein (M) and nsp3, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;
Nucleocapsid protein (N), Membrane glycoprotein (M) and nsp4, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;
Nucleocapsid protein (N), Membrane glycoprotein (M) and nsp6, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;
Nucleocapsid protein (N), Membrane glycoprotein (M) and nsp12, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp12 are selected from one of the groups consisting of (i) SEQ ID NOs: 32, 33 and 153; and (ii) SEQ ID NO: 32;
Nucleocapsid protein (N), Membrane glycoprotein (M) and nsp13, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL (neo)epitopes targeting nsp13 are selected from one of the groups consisting of (i) SEQ ID NOs: 22 and 120; and (ii) SEQ ID NO: 22;
Nucleocapsid protein (N), Membrane glycoprotein (M) and nsp14, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL epitope targeting nsp14 is SEQ ID NO: 31;
Nucleocapsid protein (N), Membrane glycoprotein (M) and nsp16, and wherein the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL epitope targeting nsp16 is SEQ ID NO: 52;

Spike glycoprotein (5), Nucleocapsid protein (N), Membrane glycoprotein (M) and Protein 3a, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL epitope targeting M is SEQ ID NO: 66; and the CTL (neo)epitopes targeting Protein 3a are selected from one of the groups consisting of (i) SEQ ID NOs: 3, 97 and 101; and (ii) SEQ ID NO: 97;
Spike glycoprotein (5), Nucleocapsid protein (N), Membrane glycoprotein (M) and nsp3, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL epitope targeting M is SEQ ID NO: 66; and the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;
Spike glycoprotein (5), Nucleocapsid protein (N), Membrane glycoprotein (M) and nsp4, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL epitope targeting M is SEQ ID NO: 66; and the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;
Spike glycoprotein (5), Nucleocapsid protein (N), Membrane glycoprotein (M) and nsp6, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL epitope targeting M is SEQ ID NO: 66; and the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;
Spike glycoprotein (5), Nucleocapsid protein (N), Membrane glycoprotein (M) and nsp12, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL epitope targeting M is SEQ ID NO: 66; and the CTL (neo)epitopes targeting nsp12 are selected from one of the groups consisting of (i) SEQ ID NOs: 32, 33 and 153; and (ii) SEQ ID NO: 32;
Spike glycoprotein (5), Nucleocapsid protein (N), Membrane glycoprotein (M) and nsp13, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL epitope targeting M is SEQ ID NO: 66; and the CTL (neo)epitopes targeting nsp13 are selected from one of the groups consisting of (i) SEQ ID NOs: 22 and 120; and (ii) SEQ ID NO: 22;
Spike glycoprotein (5), Nucleocapsid protein (N), Membrane glycoprotein (M) and nsp14, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL epitope targeting nsp14 is SEQ ID NO: 31; and
Spike glycoprotein (5), Nucleocapsid protein (N), Membrane glycoprotein (M) and nsp16, and wherein the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146; the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23; the CTL (neo)epitope targeting M is SEQ ID NO: 66; and the CTL epitope targeting nsp16 is SEQ ID NO: 52.
   9—The vaccine composition according to any one of claims 1-8, wherein the vaccine composition comprises CTL epitopes targeting at least 5, 6, 7, 8, 9, 10, or 11 proteins of SARS-CoV selected in the group consisting of Spike glycoprotein (5), Nucleocapsid protein (N), Membrane glycoprotein (M), Protein 3a, nsp3, nsp4, nsp6, nsp12, nsp13, nsp14 and nsp16; and for each proteins the CTL epitopes are selected in the following groups:
the CTL (neo)epitopes targeting S are selected from one of the groups consisting of (i) SEQ ID NOs: 34, 48, 56, 60, 70, 74, 84, 86, 91, 92, 104, and 146; (ii) SEQ ID NOs: 48, 56, 60, 70, 91, 92 and 146; and (iii) SEQ ID NOs: 70 and 146;
the CTL (neo)epitopes targeting N are selected from one of the groups consisting of (i) SEQ ID NOs: 23, 67, 75, 79, 85 and 113; (ii) SEQ ID NOs: 23 and 79; and (iii) SEQ ID NO: 23;
the CTL (neo)epitope targeting M is SEQ ID NO: 66;
the CTL (neo)epitopes targeting Protein 3a are selected from one of the groups consisting of (i) SEQ ID NOs: 3, 97 and 101; and (ii) SEQ ID NO: 97;
the CTL (neo)epitopes targeting nsp3 are selected from one of the groups consisting of (i) SEQ ID NOs: 30, 36, 49, 59, 77, 78, 90, 95 and 125; (ii) SEQ ID NOs: 30, 36, 59, 77, 78 and 125; and (iii) SEQ ID NO: 77;
the CTL (neo)epitopes targeting nsp4 are selected from one of the groups consisting of (i) SEQ ID NOs: 8, 105 and 139; (ii) SEQ ID NOs: 8 and 139; and (iii) SEQ ID NO: 8;
the CTL (neo)epitopes targeting nsp6 are selected from one of the groups consisting of (i) SEQ ID NOs: 20, 42, 76, 83, 135 and 140; (ii) SEQ ID NOs: 42, 76, 83, 135 and 140; and (iii) SEQ ID NO: 42;
the CTL (neo)epitopes targeting nsp12 are selected from one of the groups consisting of (i) SEQ ID NOs: 32, 33 and 153; and (ii) SEQ ID NO: 32;
the CTL (neo)epitopes targeting nsp13 are selected from one of the groups consisting of (i) SEQ ID NOs: 22 and 120; and (ii) SEQ ID NO: 22;
the CTL (neo)epitope targeting nsp14 is SEQ ID NO: 31; and
the CTL (neo)epitope targeting nsp16 is SEQ ID NO: 52.
   Item 10—The vaccine composition according to any one of items 1-9, wherein the vaccine composition comprises at least 1, 2, 3, 4, 5 or 6 CTL (neo)epitopes selected in one of the groups consisting of (i) SEQ ID NOs: 20, 23, 32, 36, 42, 56, 59, 60, 76, 79, 85, 91, 95, 97, 125, 140 and 146; (ii) SEQ ID NOs: 23, 32, 36, 42, 56, 59, 60, 76, 79, 91, 97, 125, 140 and 146; and (iii) SEQ ID NOs: 23, 32, 42, 97 and 146.
   Item 11—The vaccine composition according to any one of items 1-10, wherein the vaccine composition comprises at least 5, 6, 7, 8, 9, 10, 11, or 12 CTL (neo)epitopes selected from SEQ ID NOs: 8, 22, 23, 31, 32, 42, 52, 66, 77, 97 and 146.
   Item 12—The vaccine composition according to item 11, wherein the vaccine composition comprises CTL (neo)epitopes of SEQ ID NOs: 70 and/or 146; 23, and 66, and at least 2, 3, 4, 5, 6, 7 or 8 CTL (neo)epitopes selected from SEQ ID NOs: 8, 22, 31, 32, 42, 52, 77 and 97.
   Item 13—The vaccine composition according to item 11, wherein the vaccine composition comprises CTL (neo)epitopes of SEQ ID NOs: 8, 22, 23, 31, 32, 42, 52, 66, 70, 77, 97 and 146.
   Item 14—The vaccine composition according to item 11, wherein the vaccine composition comprises CTL (neo)epitopes of SEQ ID NOs: 8, 22, 23, 31, 32, 42, 52, 66, 70, 77, 97 and 146 and a T helper peptide, especially PADRE (aKXVAAWTLKAAa with X and a respectively indicating cyclohexylalanine and d-alanine).
   Item 15—A vaccine composition as defined in any one of the items 1-14 for use for preventing or treating an infection by a severe acute respiratory syndrome-related coronavirus (SARS-CoV).
   Item 16—The vaccine composition for use according to item 15, wherein the SARS-CoV is selected from the group consisting of SARS-CoV1, SARS-CoV2 or MERS-CoV virus, preferably SARS-CoV2.
   Item 17—The vaccine composition for use according to any one of item 15-16, wherein the subject to be treated is a subject aged 65 years or older, a subject having a cancer or having had a cancer, a subject being obese (In particular with severe obesity (body mass index [BMI] of 40 or higher [CDC-HCSP BMI>30]), a subject being diabetic, a subject having a hypertension, a subject having a sarcoidosis, a subject being immunocompromised, a subject who lives in a nursing home or long-term care facility, a subject with chronic lung disease or moderate to severe asthma, lung fibrosis, a subject who has serious heart conditions, a subject with chronic kidney disease undergoing dialysis and/or a subject with liver diseases; and/or a subject being HLA-A2.

1.-26. (canceled)

27. A vaccine composition comprising:
CTL neoepitopes of SEQ ID NOs: 70 and/or 146;
CTL epitope of SEQ ID NO: 23;
CTL neoepitope of SEQ ID NO: 66; and
at least 2, 3, 4, 5, 6, 7 or 8 CTL (neo)epitopes selected from SEQ ID NOs: 8, 22, 31, 32, 42, 52, 77 and 97.

28. The vaccine composition according to claim 27, wherein the composition further comprises at least 1 HTL peptide/epitope.

29. The vaccine composition according to claim 27, wherein the composition comprises aKXVAAWTLKAAa with X and a respectively indicating cyclohexylalanine and d-alanine.

30. The vaccine composition according to claim 27, wherein the vaccine composition comprises CTL (neo)epitopes of SEQ ID NOs: 8, 22, 23, 31, 32, 42, 52, 66, 70, 77, 97 and 146.

31. The vaccine composition according to claim 27, wherein the vaccine composition comprises CTL (neo)epitopes of SEQ ID NOs: 8, 22, 23, 31, 32, 42, 52, 66, 70, 77, 97 and 146 and a T helper peptide.

32. The vaccine composition according to claim 27, wherein the vaccine composition further comprises an adjuvant.

33. The vaccine composition according to claim 32, wherein the adjuvant is a mixture of mineral oil and mannide mono-oleate.

34. The vaccine composition according to claim 27, wherein each peptide is present at a concentration of 0.01 mg/ml to 1 g/ml.

35. The vaccine composition according to claim 27, wherein the CTL (neo)epitopes are each at a dose of between 1 and 100 μg.

36. The vaccine composition according to claim 28, wherein the T helper peptide are at a dose of between 1 and 100 μg.

37. A method of treating a severe acute respiratory syndrome-related coronavirus (SARS-CoV) infection comprising administering a vaccine composition according to claim 27 to a subject in need of treatment.

38. The method according to claim 37, wherein the SARS-CoV is selected from the group consisting of SARS-CoV1, SARS-CoV2 and MERS-CoV virus.

39. The method according to claim 37, wherein the SARS-CoV is SARS-CoV2 virus.

40. The method according to claim 37, wherein the subject to be treated is a subject aged 65 years or older.

41. The method according to claim 37, wherein the subject to be treated is a subject having a cancer or having had a cancer.

42. The method according to claim 37, wherein the subject to be treated is a subject that is obese.

43. The method according to claim 37, wherein the subject to be treated is a subject that is diabetic, has hypertension, has a sarcoidosis, is immunocompromised, lives in a nursing home or long-term care facility, has chronic lung disease, has moderate to severe asthma, has lung fibrosis, has a serious heart condition, has chronic kidney disease and is undergoing dialysis, or has liver disease.

44. The method according to claim 37, wherein the subject to be treated is a subject being HLA-A2 positive.


SEQUENCE LISTING (176 sequences)

SEQ ID NO: 1 - protein, 9 aa, artificial sequence
  peptide
ALNTLVKQL

SEQ ID NO: 2 - protein, 9 aa, artificial sequence
  peptide
ALNTPKDHI

SEQ ID NO: 3 - protein, 9 aa, artificial sequence
  peptide
ALSKGVHFV

SEQ ID NO: 4 - protein, 9 aa, artificial sequence
  peptide
AMYTPHTVL

SEQ ID NO: 5 - protein, 9 aa, artificial sequence
  peptide
CLEASFNYL

SEQ ID NO: 6 - protein, 9 aa, artificial sequence
  peptide
FIAGLIAIV

SEQ ID NO: 7 - protein, 9 aa, artificial sequence
  peptide
FLAHIQWMV

SEQ ID NO: 8 - protein, 9 aa, artificial sequence
  peptide
FLLNKEMYL

SEQ ID NO: 9 - protein, 9 aa, artificial sequence
  peptide
FLTENLLLY

SEQ ID NO: 10 - protein, 9 aa, artificial sequence
  peptide
FQFCNDPFL

SEQ ID NO: 11 - protein, 9 aa, artificial sequence
  peptide
FVLAAVYRI

SEQ ID NO: 12 - protein, 9 aa, artificial sequence
  peptide
GKQGNFKNL

SEQ ID NO: 13 - protein, 9 aa, artificial sequence
  peptide
GLFKDCSKV

SEQ ID NO: 14 - protein, 9 aa, artificial sequence
  peptide
GLMWLSYFI

SEQ ID NO: 15 - protein, 9 aa, artificial sequence
  peptide
GMSRIGMEV

SEQ ID NO: 16 - protein, 10 aa, artificial sequence
  peptide
GYQPYRVVVL

SEQ ID NO: 17 - protein, 9 aa, artificial sequence
  peptide
ILLLDQALV

SEQ ID NO: 18 - protein, 9 aa, artificial sequence
  peptide
ILLNKHIDA

SEQ ID NO: 19 - protein, 9 aa, artificial sequence
  peptide
KIADYNYKL

SEQ ID NO: 20 - protein, 9 aa, artificial sequence
  peptide
KLKDCVMYA

SEQ ID NO: 21 - protein, 10 aa, artificial sequence
  peptide
KLPDDFTGCV

SEQ ID NO: 22 - protein, 9 aa, artificial sequence
  peptide
KLSYGIATV

SEQ ID NO: 23 - protein, 9 aa, artificial sequence
  peptide
LALLLLDRL

SEQ ID NO: 24 - protein, 9 aa, artificial sequence
  peptide
LITGRLQSL

SEQ ID NO: 25 - protein, 9 aa, artificial sequence
  peptide
LLLDRLNQL

SEQ ID NO: 26 - protein, 9 aa, artificial sequence
  peptide
LLLTILTSL

SEQ ID NO: 27 - protein, 9 aa, artificial sequence
  peptide
LLSAGIFGA

SEQ ID NO: 28 - protein, 9 aa, artificial sequence
  peptide
LMWLIINLV

SEQ ID NO: 29 - protein, 9 aa, artificial sequence
  peptide
LQLPQGTTL

SEQ ID NO: 30 - protein, 9 aa, artificial sequence
  peptide
MLAKALRKV

SEQ ID NO: 31 - protein, 9 aa, artificial sequence
  peptide
MMISAGFSL

SEQ ID NO: 32 - protein, 9 aa, artificial sequence
  peptide
NLIDSYFVV

SEQ ID NO: 33 - protein, 9 aa, artificial sequence
  peptide
NLLKDCPAV

SEQ ID NO: 34 - protein, 9 aa, artificial sequence
  peptide
NLNESLIDL

SEQ ID NO: 35 - protein, 9 aa, artificial sequence
  peptide
PYRVVVLSF

SEQ ID NO: 36 - protein, 9 aa, artificial sequence
  peptide
QLEMELTPV

SEQ ID NO: 37 - protein, 9 aa, artificial sequence
  peptide
RIMTWLDMV

SEQ ID NO: 38 - protein, 9 aa, artificial sequence
  peptide
RLNEVAKNL

SEQ ID NO: 39 - protein, 9 aa, artificial sequence
  peptide
RLNQLESKM

SEQ ID NO: 40 - protein, 9 aa, artificial sequence
  peptide
SIIAYTMSL

SEQ ID NO: 41 - protein, 9 aa, artificial sequence
  peptide
SLPGVFCGV

SEQ ID NO: 42 - protein, 9 aa, artificial sequence
  peptide
SMWALIISV

SEQ ID NO: 43 - protein, 10 aa, artificial sequence
  peptide
TLACFVLAAV

SEQ ID NO: 44 - protein, 9 aa, artificial sequence
  peptide
TLMNVLTLV

SEQ ID NO: 45 - protein, 9 aa, artificial sequence
  peptide
VLNDILSRL

SEQ ID NO: 46 - protein, 9 aa, artificial sequence
  peptide
VQMAPISAM

SEQ ID NO: 47 - protein, 9 aa, artificial sequence
  peptide
VSPTKLNDL

SEQ ID NO: 48 - protein, 9 aa, artificial sequence
  peptide
VVFLHVTYV

SEQ ID NO: 49 - protein, 9 aa, artificial sequence
  peptide
WLMWLIINL

SEQ ID NO: 50 - protein, 9 aa, artificial sequence
  peptide
YLDAYNMMI

SEQ ID NO: 51 - protein, 9 aa, artificial sequence
  peptide
YLNSTNVTI

SEQ ID NO: 52 - protein, 9 aa, artificial sequence
  peptide
YLNTLTLAV

SEQ ID NO: 53 - protein, 9 aa, artificial sequence
  peptide
YLQPRTFLL

SEQ ID NO: 54 - protein, 9 aa, artificial sequence
  peptide
YLYALVYFL

SEQ ID NO: 55 - protein, 9 aa, artificial sequence
  peptide
YTMADLVYA

SEQ ID NO: 56 - protein, 9 aa, artificial sequence
  peptide
ALNTLVKQV

SEQ ID NO: 57 - protein, 9 aa, artificial sequence
  peptide
ALNTPKDHV

SEQ ID NO: 58 - protein, 9 aa, artificial sequence
  peptide
ALYTPHTVV

SEQ ID NO: 59 - protein, 9 aa, artificial sequence
  peptide
CLEASFNYV

SEQ ID NO: 60 - protein, 9 aa, artificial sequence
  peptide
FLAGLIAIV

SEQ ID NO: 61 - protein, 9 aa, artificial sequence
  peptide
FLLNKEMYV

SEQ ID NO: 62 - protein, 9 aa, artificial sequence
  peptide
FLTENLLLV

SEQ ID NO: 63 - protein, 9 aa, artificial sequence
  peptide
FLFCNDPFV

SEQ ID NO: 64 - protein, 9 aa, artificial sequence
  peptide
FLLAAVYRV

SEQ ID NO: 65 - protein, 9 aa, artificial sequence
  peptide
GLQGNFKNV

SEQ ID NO: 66 - protein, 9 aa, artificial sequence
  peptide
GLMWLSYFV

SEQ ID NO: 67 - protein, 9 aa, artificial sequence
  peptide
GLSRIGMEV

SEQ ID NO: 68 - protein, 9 aa, artificial sequence
  peptide
GLQPYRVVV

SEQ ID NO: 69 - protein, 9 aa, artificial sequence
  peptide
ILLNKHIDV

SEQ ID NO: 70 - protein, 9 aa, artificial sequence
  peptide
KLADYNYKV

SEQ ID NO: 71 - protein, 9 aa, artificial sequence
  peptide
KLKDCVMYV

SEQ ID NO: 72 - protein, 9 aa, artificial sequence
  peptide
KLPDDFTGV

SEQ ID NO: 73 - protein, 9 aa, artificial sequence
  peptide
LLLLLLDRV

SEQ ID NO: 74 - protein, 9 aa, artificial sequence
  peptide
LLTGRLQSV

SEQ ID NO: 75 - protein, 9 aa, artificial sequence
  peptide
LLLDRLNQV

SEQ ID NO: 76 - protein, 9 aa, artificial sequence
  peptide
LLLTILTSV

SEQ ID NO: 77 - protein, 9 aa, artificial sequence
  peptide
LLSAGIFGV

SEQ ID NO: 78 - protein, 9 aa, artificial sequence
  peptide
LLWLIINLV

SEQ ID NO: 79 - protein, 9 aa, artificial sequence
  peptide
LLLPQGTTV

SEQ ID NO: 80 - protein, 9 aa, artificial sequence
  peptide
MLISAGFSV

SEQ ID NO: 81 - protein, 9 aa, artificial sequence
  peptide
NLNESLIDV

SEQ ID NO: 82 - protein, 9 aa, artificial sequence
  peptide
PLRVVVLSV

SEQ ID NO: 83 - protein, 9 aa, artificial sequence
  peptide
RLMTWLDMV

SEQ ID NO: 84 - protein, 9 aa, artificial sequence
  peptide
RLNEVAKNV

SEQ ID NO: 85 - protein, 9 aa, artificial sequence
  peptide
RLNQLESKV

SEQ ID NO: 86 - protein, 9 aa, artificial sequence
  peptide
SLIAYTMSV

SEQ ID NO: 87 - protein, 9 aa, artificial sequence
  peptide
SLWALIISV

SEQ ID NO: 88 - protein, 9 aa, artificial sequence
  peptide
TLACFVLAV

SEQ ID NO: 89 - protein, 9 aa, artificial sequence
  peptide
VLNDILSRV

SEQ ID NO: 90 - protein, 9 aa, artificial sequence
  peptide
VLMAPISAV

SEQ ID NO: 91 - protein, 9 aa, artificial sequence
  peptide
VLPTKLNDV

SEQ ID NO: 92 - protein, 9 aa, artificial sequence
  peptide
VLFLHVTYV

SEQ ID NO: 93 - protein, 9 aa, artificial sequence
  peptide
WLMWLIINV

SEQ ID NO: 94 - protein, 9 aa, artificial sequence
  peptide
YLDAYNMMV

SEQ ID NO: 95 - protein, 9 aa, artificial sequence
  peptide
YLNSTNVTV

SEQ ID NO: 96 - protein, 9 aa, artificial sequence
  peptide
YLQPRTFLV

SEQ ID NO: 97 - protein, 9 aa, artificial sequence
  peptide
YLYALVYFV

SEQ ID NO: 98 - protein, 9 aa, artificial sequence
  peptide
YLMADLVYV

SEQ ID NO: 99 - protein, 9 aa, artificial sequence
  peptide
ASNTLVKQL

SEQ ID NO: 100 - protein, 9 aa, artificial sequence
  peptide
AVNTPKDHE

SEQ ID NO: 101 - protein, 9 aa, artificial sequence
  peptide
AVSKGVHFI

SEQ ID NO: 102 - protein, 9 aa, artificial sequence
  peptide
AGYTPHTVV

SEQ ID NO: 103 - protein, 9 aa, artificial sequence
  peptide
CVEASFNYL

SEQ ID NO: 104 - protein, 9 aa, artificial sequence
  peptide
FMAGLIAIV

SEQ ID NO: 105 - protein, 9 aa, artificial sequence
  peptide
FTAHIQWMV

SEQ ID NO: 106 - protein, 9 aa, artificial sequence
  peptide
FTLNKEMYL

SEQ ID NO: 107 - protein, 9 aa, artificial sequence
  peptide
FITENLLLF

SEQ ID NO: 108 - protein, 9 aa, artificial sequence
  peptide
FSFCNDPFL

SEQ ID NO: 109 - protein, 9 aa, artificial sequence
  peptide
FLLAAVYRF

SEQ ID NO: 110 - protein, 9 aa, artificial sequence
  peptide
GVQGNFKNL

SEQ ID NO: 111 - protein, 9 aa, artificial sequence
  peptide
GFFKDCSKG

SEQ ID NO: 112 - protein, 9 aa, artificial sequence
  peptide
GLMWLSYFT

SEQ ID NO: 113 - protein, 9 aa, artificial sequence
  peptide
GTSRIGMEV

SEQ ID NO: 114 - protein, 10 aa, artificial sequence
  peptide
GFQPYRVVVL

SEQ ID NO: 115 - protein, 9 aa, artificial sequence
  peptide
IPLLDQALV

SEQ ID NO: 116 - protein, 9 aa, artificial sequence
  peptide
ILLNKHIDG

SEQ ID NO: 117 - protein, 9 aa, artificial sequence
  peptide
KHADYNYKL

SEQ ID NO: 118 - protein, 9 aa, artificial sequence
  peptide
KIKDCVMYV

SEQ ID NO: 119 - protein, 10 aa, artificial sequence
  peptide
KAPDDFTGCV

SEQ ID NO: 120 - protein, 9 aa, artificial sequence
  peptide
KLSYGIATE

SEQ ID NO: 121 - protein, 9 aa, artificial sequence
  peptide
LCLLLLDRI

SEQ ID NO: 122 - protein, 9 aa, artificial sequence
  peptide
LATGRLQSI

SEQ ID NO: 123 - protein, 9 aa, artificial sequence
  peptide
LCLDRLNQR

SEQ ID NO: 124 - protein, 9 aa, artificial sequence
  peptide
LPLTILTSK

SEQ ID NO: 125 - protein, 9 aa, artificial sequence
  peptide
LVSAGIFGV

SEQ ID NO: 126 - protein, 9 aa, artificial sequence
  peptide
LAWLIINLV

SEQ ID NO: 127 - protein, 9 aa, artificial sequence
  peptide
LGLPQGTTE

SEQ ID NO: 128 - protein, 9 aa, artificial sequence
  peptide
MPAKALRKV

SEQ ID NO: 129 - protein, 9 aa, artificial sequence
  peptide
MAISAGFSV

SEQ ID NO: 130 - protein, 9 aa, artificial sequence
  peptide
NAIDSYFVL

SEQ ID NO: 131 - protein, 9 aa, artificial sequence
  peptide
NWLKDCPAI

SEQ ID NO: 132 - protein, 9 aa, artificial sequence
  peptide
NWNESLIDD

SEQ ID NO: 133 - protein, 9 aa, artificial sequence
  peptide
PLRVVVLSP

SEQ ID NO: 134 - protein, 9 aa, artificial sequence
  peptide
QEEMELTPI

SEQ ID NO: 135 - protein, 9 aa, artificial sequence
  peptide
RTMTWLDMI

SEQ ID NO: 136 - protein, 9 aa, artificial sequence
  peptide
RPNEVAKNT

SEQ ID NO: 137 - protein, 9 aa, artificial sequence
  peptide
RLNQLESKT

SEQ ID NO: 138 - protein, 9 aa, artificial sequence
  peptide
SPIAYTMSV

SEQ ID NO: 139 - protein, 9 aa, artificial sequence
  peptide
SAPGVFCGV

SEQ ID NO: 140 - protein, 9 aa, artificial sequence
  peptide
SAWALIISV

SEQ ID NO: 141 - protein, 10 aa, artificial sequence
  peptide
TNACFVLAAA

SEQ ID NO: 142 - protein, 9 aa, artificial sequence
  peptide
TAMNVLTLV

SEQ ID NO: 143 - protein, 9 aa, artificial sequence
  peptide
VANDILSRL

SEQ ID NO: 144 - protein, 9 aa, artificial sequence
  peptide
VYMAPISAM

SEQ ID NO: 145 - protein, 9 aa, artificial sequence
  peptide
VSPTKLNDG

SEQ ID NO: 146 - protein, 9 aa, artificial sequence
  peptide
VSFLHVTYV

SEQ ID NO: 147 - protein, 9 aa, artificial sequence
  peptide
WYMWLIINV

SEQ ID NO: 148 - protein, 9 aa, artificial sequence
  peptide
WCYPKCDRS

SEQ ID NO: 149 - protein, 9 aa, artificial sequence
  peptide
YANSTNVTI

SEQ ID NO: 150 - protein, 9 aa, artificial sequence
  peptide
YRNTLTLAV

SEQ ID NO: 151 - protein, 9 aa, artificial sequence
  peptide
YNNSTNVTS

SEQ ID NO: 152 - protein, 9 aa, artificial sequence
  peptide
YGYALVYFA

SEQ ID NO: 153 - protein, 9 aa, artificial sequence
  peptide
YLMADLVYT

SEQ ID NO: 154 - protein, 23 aa, artificial sequence
  peptide
NSNNLDSKVGGNYNYLYRLFRKS

SEQ ID NO: 155 - protein, 11 aa, artificial sequence
  peptide
NNLDSKVGGNY

SEQ ID NO: 156 - protein, 12 aa, artificial sequence
  peptide
NYNYLYRLFRKS

SEQ ID NO: 157 - protein, 28 aa, artificial sequence
  peptide
NYNYLYRLFRKSNLKPFERDISTEIYQA

SEQ ID NO: 158 - protein, 15 aa, artificial sequence
  peptide
YQAGSTPCNGVEGFN

SEQ ID NO: 159 - protein, 25 aa, artificial sequence
  peptide
EGFNCYFPLQSYGFQPTNGVGYQPY

SEQ ID NO: 160 - protein, 16 aa, artificial sequence
  peptide
PLQSYGFQPTNGVGYQ

SEQ ID NO: 161 - protein, 24 aa, artificial sequence
  peptide
RVVVLSFELLHAPATVCPGKKSTN

SEQ ID NO: 162 - protein, 35 aa, artificial
  peptide
  MISC_FEATURE (1)..(1): XAA = D-ALANINE
  MISC_FEATURE (3)..(3): XAA = L-Cyclohexylalanine
  MISC_FEATURE (12)..(12): XAA = D-ALANINE
XKXVAAWTLKAXNSNNLDSKVGGNYNYLYRLFRKS

SEQ ID NO: 163 - protein, 23 aa, artificial
  peptide
  MISC_FEATURE (1)..(1): XAA = D-ALANINE
  MISC_FEATURE (3)..(3): XAA = L-Cyclohexylalanine
  MISC_FEATURE (12)..(12): XAA = D-ALANINE
XKXVAAWTLKAXNNLDSKVGGNY

SEQ ID NO: 164 - protein, 24 aa, artificial
  peptide
  MISC_FEATURE (1)..(1): XAA = D-ALANINE
  MISC_FEATURE (3)..(3): XAA = L-Cyclohexylalanine
  MISC_FEATURE (12)..(12): XAA = D-ALANINE
XKXVAAWTLKAXNYNYLYRLFRKS

SEQ ID NO: 165 - protein, 40 aa, artificial
  peptide
  MISC_FEATURE (1)..(1): XAA = D-ALANINE
  MISC_FEATURE (3)..(3): XAA = L-Cyclohexylalanine
  MISC_FEATURE (12)..(12): XAA = D-ALANINE
XKXVAAWTLKAXNYNYLYRLFRKSNLKPFERDISTEIYQA

SEQ ID NO: 166 - protein, 27 aa, artificial
  peptide
  MISC_FEATURE (1)..(1): XAA = D-ALANINE
  MISC_FEATURE (3)..(3): XAA = L-Cyclohexylalanine
  MISC_FEATURE (12)..(12): XAA = D-ALANINE
AKXVAAWTLKAAYQAGSTPCNGVEGFN

SEQ ID NO: 167 - protein, 37 aa, artificial
  peptide
  MISC_FEATURE (1)..(1): XAA = D-ALANINE
  MISC_FEATURE (3)..(3): XAA = L-Cyclohexylalanine
  MISC_FEATURE (12)..(12): XAA = D-ALANINE
AKXVAAWTLKAAEGFNCYFPLQSYGFQPTNGVGYQPY

SEQ ID NO: 168 - protein, 28 aa, artificial
  peptide
  MISC_FEATURE (1)..(1): XAA = D-ALANINE
  MISC_FEATURE (3)..(3): XAA = L-Cyclohexylalanine
  MISC_FEATURE (12)..(12): XAA = D-ALANINE
XKXVAAWTLKAXPLQSYGFQPTNGVGYQ

SEQ ID NO: 169 - protein, 36 aa, artificial
  peptide
  MISC_FEATURE (1)..(1): XAA = D-ALANINE
  MISC_FEATURE (3)..(3): XAA = L-Cyclohexylalanine
  MISC_FEATURE (12)..(12): XAA = D-ALANINE
XKXVAAWTLKAXRVVVLSFELLHAPATVCPGKKSTN

SEQ ID NO: 170 - protein, 13 aa, artificial sequence
  PADRE
  MISC_FEATURE (1)..(1): XAA = D-ALANINE
  MISC_FEATURE (3)..(3): XAA = L-Cyclohexylalanine
  MISC_FEATURE (12)..(12): XAA = D-ALANINE
  MISC_FEATURE (13)..(13): XAA = D-ALANINE
XKXVAAWTLKAAX

SEQ ID NO: 171 - protein, 9 aa, artificial
  MPS-216
KVFGSLAFV

SEQ ID NO: 172 - protein, 9 aa, artificial
  MPS-102
  MISC_FEATURE (3)..(3): Xaa is alpha-aminobutyric acid
KLXPVQLWV

SEQ ID NO: 173 - protein, 9 aa, artificial
  MPS-112
RLLQETELV

SEQ ID NO: 174 - protein, 10 aa, artificial
  MPS-106
YLQLVFGIEV

SEQ ID NO: 175 - protein, 9 aa, artificial
  MPS-213
LLTFWNPPV

SEQ ID NO: 176 - protein, 9 aa, artificial
  MPS-103
SMPPPGTRV